(19) United States
(12) Patent Application Publication
(10) Pub. No.: US 2025/0032537 A1
(43) Pub. Date: Jan. 30, 2025

(54) PROCESS FOR REMOVING Pb2+ IONS FROM BODILY FLUIDS USING METAL TITANATE ION EXCHANGERS

(71) Applicant: UOP LLC, Des Plaines, IL (US)

(72) Inventors: Gregory J. Lewis, Sunnyvale, CA (US); Paulina Jakubczak, Elk Grove Village, IL (US); Mimoza Sylejmani-Rekaliu, Bensenville, IL (US); William Sheets, Glenview, IL (US); Evgeny Kolev, Arlington Heights, IL (US); Rodrigo Lobo, Des Plaines, IL (US); Keith Lehuta, Chicago, IL (US); Subhadra Chudasama, Northbrook, IL (US)

(21) Appl. No.: 18/782,880

(22) Filed: Jul. 24, 2024

Related U.S. Application Data
(60) Provisional application No. 63/528,801, filed on Jul. 25, 2023.

(51) Int. Cl. A61K 33/26; A61K 9/00; A61K 33/00; A61K 33/06; A61P 39/04; B01D 15/36; B01J 39/02; B01J 39/09

(52) U.S. Cl. CPC A61K 33/26; A61K 9/0053; A61K 33/00; A61K 33/06; A61P 39/04; B01D 15/362; B01J 39/02; B01J 39/09

(57) ABSTRACT

A process for removing Pb²⁺ ions from fluids, such as gastrointestinal fluids is described. The process involves contacting gastrointestinal fluid with a particulate metal titanate ion exchanger represented by the empirical formula:

AₘTiₓM_{y}O_{z}

where A is an exchangeable cation selected from the group consisting of potassium ion, sodium ion, lithium ion, calcium ion, magnesium ion, hydronium ion, and mixtures thereof, and M is optionally at least one framework metal selected from niobium (5+), zirconium (4+), tin (4+), iron (3+), iron (2+), cobalt (2+), and manganese (2+). The particulate metal titanate ion exchangers of the present disclosure are synthesized in the presence of complexing agents, including at least one multihydroxyl-containing complexing agent (MHCA), which facilitate metal transport and incorporation while imparting beneficial properties such as large aggregate size, well-behaved particle size distribution, and macroporosity.

### FIELD OF THE INVENTION

The present disclosure relates to intracorporeal processes for removing Pb²⁺ ions from bodily fluids, especially gastrointestinal fluids, using particulate metal titanate ion exchangers. A gastrointestinal fluid is contacted directly with a particulate metal titanate ion exchanger composition, which removes Pb²⁺ toxins present in the fluid. The present disclosure also relates to particulate metal titanate ion exchangers synthesized in the presence of complexing agents, including at least one multihydroxyl-containing complexing agent (MHCA), which facilitate metal incorporation and impart favorable properties, including large aggregate size, more uniform particle size distribution and macroporosity.

### BACKGROUND

Lead has become widely distributed in the biosphere only in the past few thousand years, entirely as the result of human activity. Once introduced into the environment, lead persists. Investigations of human skeletal remains indicate that the body lead burden of today's populations is 500-1000 times greater than that of their pre-industrial counterparts (See NE J. Med., vol. 326, no. 19, pp. 1293-1294, 1992). By far the largest contributor to global environmental lead contamination was the use of lead in gasoline, mainly between 1965 and 1990 (See Environ. Health Perspect., vol. 110, no. 7, pp. 721-728, 2002). With lead now removed from gasoline across the developed world, blood lead levels (BLLs) in the US and worldwide are expected to continue their slow decline (See Am. J. Med., vol. 129, pp. 1213-1218, 2016). However, hot spots from smelting, mining, aging houses, water lines, highways, and metal recycling operations-some of them ongoing and others the legacy of the past-remain significant problems.

Despite a century of accumulated evidence about its danger to the health of children, lead often continues to be added to paints, pigments, toys, traditional medications, cosmetics, and other consumer products, especially as manufacturing shifts to developing countries that lack environmental and product content controls and policies. Dust from lead-based paint remains a significant source of exposure (See J. Pediatrics, vol. 140, no. 1, pp. 40-47, 2002). Soils in older areas of cities are often highly contaminated by lead, owing to past use of lead additives in gasoline and industrial sources.

Soils are not passive sources and periodic re-suspension of fine lead-contaminated soil dust particulates create seasonal variations of lead exposure for urban dwellers (See Atmos. Environ., vol. 49, pp. 302-310, 2012). Furthermore, there exists a coupling between inhaled and ingested lead. Inhalation studies examining lead particles (˜1 μm) deposited in the back of the nose have demonstrated that they are typically swallowed over time. In addition, particles deposited in the tracheobronchial region may be cleared from the lungs via the mucociliary escalator, reaching the gastrointestinal system via the esophagus (See Radiat. Prot. Dosim., vol. 127, pp. 31-34, 2007).

A change in water supply utilizing old, contaminated infrastructure brought lead-contaminated water to Flint, Michigan where significant elevation of blood lead levels (BLLs) was observed in children (See Am. J. Public Health, vol. 106, no. 2, pp. 283-290, 2016). A report released by the Natural Resources Defense Council in 2018 details how many other communities around the country are failing to adequately ensure that their water supplies remain free of lead (See E. Olson and K. P. Fedinick, Natural Resources Defense Council, New York, NY, 2016). Experts have estimated that 6 to 10 million lead service lines are being used in the US, serving 15 to 22 million Americans, most of which were installed at least 50 years ago (See J. Am. Water Works Assoc., vol. 108, no. 4, pp. E182-E191, 2016).

Lead is highly toxic, it can harm the brain, kidneys, bone marrow and other bodily systems, especially those of young children. The Third National Health and Nutritional Examination (NHANES III, Phase 2, 1991-1994) found that 4.4% of children under 6 years old in the United States had BLLs above 10 μg/dL, a level that is considered “poisoned.” (See Eliminating Childhood Lead Poisoning: A Federal Strategy Targeting Lead Paint Hazards; President's Task Force on Environmental Health Risks and Safety Risks to Children, February 2000). Exposure via lead-containing paint is the main culprit, with the impact greater on children from low-income and minority families living in older housing, as 16% of these children are poisoned compared to the national average of 4.4% for all children (See Morbidity and Mortality Weekly Report, U.S. Department of Health and Human Services/Public Health Service, Vol 46, No. 7, Feb. 21, 1997, p. 141-146).

As BLLs increase in children, ill effects and morbidity associated with lead poisoning increases, including reduced IQ, decreased hearing, and decreased growth. Moreover, behavioral problems in children can be observed at BLL=10 μg/dL, impaired nerve function may occur at BLL=20 μg/dL, reduced vitamin C metabolism may occur at BLL=30 μg/dL, damage to hematopoiesis may occur at BLL=40 μg/dL, severe stomach cramps may occur at BLL above 50 μg/dL, and severe brain and kidney damage and severe anemia may occur at BLL between 50-100 μg/dL. Even low-level elevations in children's blood lead concentrations, such as at concentrations below 5 μg/dL, can result in decrements in cognitive functions, as measured by IQ scores and academic performance (See Public Health Rep., vol. 115, pp. 521-529, 2000; and Environ. Health Perspect., vol. 113, pp. 894-899, 2005).

For a given level of exposure, lead-associated IQ losses are proportionately greater at the lowest blood lead concentrations. While the average IQ decrease associated with an increase in blood lead concentration from <1 to 30 μg/dL was 9.2 IQ points, the decrease associated with an increase in blood lead concentration from <1 to 10 μg/dL was 6.2 IQ points. The population impact of lead on intellectual abilities is substantial, as lead toxicity accounts for an estimated total loss of 23 million IQ points among a 6-year cohort of contemporary US children (See Environ. Health Perspect., vol. 120, pp. 501-507, 2012). There is also an inverse relationship between early childhood exposure to lead and performance on tests of cognitive function and behavior 10 and 20 years after the blood lead levels were measured (See Pediatrics, vol. 90, pp. 855-861, 1992). Additionally, early exposures have been linked to increased rates of hyperactivity, inattentiveness, failure to graduate from high school, conduct disorder, juvenile delinquency, drug use and incarceration (See PLos Medicine, vol. 5, p. e101, 2008).

With respect to kidney damage, a study that examined the NHANES III (1988-1994) data reported that among 769 adolescents with a median blood lead concentration of 1.5 μg/dL (15 ppb), a doubling of the concentration of lead in blood led to a significant reduction in the glomerular filtration rate (See Arch Intern Med., vol. 170, pp. 75-82, 2010).

Numerous experimental and epidemiological studies suggest that lead exposure is a risk factor for cardiovascular disease (CVD) and mortality. While a BLL in adults of 5 μg/dL is considered to be elevated, a recent study concluded that low-level environmental lead exposure (i.e., <5 μg/dL) is a risk factor for CVD mortality (See Lancet Public Health, vol. 3, pp. e177-184, 2018). The authors tracked NHANES III (1988-1994) participants through 2011 (˜14,000 adults) and examined the relationship between lower levels of lead, down to a BLL of 1 μg/dL, and CVD and ischemic heart disease mortality. Analysis of the data suggested that ˜400,000 US deaths annually are attributable to lead exposure in subjects having cardiovascular disease and/or diabetes, highlighting the potential risk factor of adult BLL concentrations of 1-5 μg/dL.

Chelation therapy has been used to remove lead from the blood. An optimal chelating drug should increase lead excretion, be administered easily, be affordable and safe. However, lead-chelate complexes may persist in tissues where the binding occurred, or may be redistributed to other tissues. Increased symptoms commonly reported with aggressive initiation of chelation therapies are cited as a contraindication to any use of chelators. Chelating agents effectively remove lead in the blood and are indicated for acutely affected patients with a BLL of greater than 45 μg/dL and are administered in consultation with a specialist (See Environmental Health Perspectives, vol. 115, no. 3, pp. 463-471, 2007). Chelating agents used to treat lead poisoning include intravenous (IV) calcium disodium edetate (CaNa₂EDTA), intramuscular dimercaprol, and oral 2,3-dimercaptosuccinic acid (DMSA, also known as succimer).

EDTA, ethylenediaminetetraacetic acid, is used in the CaNa₂EDTA form for chelation of lead to avoid hypocalcemia and possibly death due to its capability to bind calcium. It can also bind other bioavailable cations such as zinc, copper, and iron (See Int. J. Environ. Res. Public Health, vol. 7, pp. 2745-2788, 2010). It is often used in the treatment of severe lead poisoning and is administered intravenously in a hospital setting because substantial monitoring is required including renal function, cardiac activity, and daily phlebotomy to monitor serum electrolytes. Pain and swelling may result from the parenteral administration of CaNa₂EDTA, while other adverse effects include fever, nausea, vomiting, and nephrotoxicity, such as microscopic hematuria and proteinuria. CaNa₂EDTA has been reported to spread lead to other tissues and can increase lead concentrations in the central nervous system and may cause encephalopathy. After a single dose of CaNa₂EDTA, urinary lead levels increase, blood lead levels decrease, and brain lead levels increase significantly due to redistribution of lead into the brain.

DMSA is indicated for BLLs of greater than 45 μg/dL for treatment of acute lead poisoning situations and does not require administration in a hospital. Oral DMSA is dosed typically at 30 mg/kg/day for 5 days (See Med. Toxicol. Adverse Drug Exp., vol. 3, pp. 499-504, 1988), often followed by a 14-day course of 20 mg/kg/day, a treatment regimen that increases urine lead excretion and reduces blood lead concentrations significantly. Drug-induced neutropenia can occur, so weekly blood count is recommended, and treatment should be discontinued if neutrophil counts get too low. Adverse effects of DMSA treatment include nausea, vomiting, diarrhea, loose stool, metallic taste that are experienced singly or together by 12% of children and 21% of adults (See U.S. Pat. No. 11,083,748). Back pain, abdominal cramps, chills, and flu-like symptoms have also been reported in 5% of children and 16% of adults.

Dimercaprol, also known as British Anti-Lewisite (BAL) was originally developed as an experimental antidote to Lewisite, an arsenic-based poison gas. To administer dimercaprol to a patient, it is dissolved in peanut oil and injected into the muscles, a procedure not well tolerated by children. Dimercaprol, a small molecule, can penetrate the blood-brain barrier and can be used to treat encephalopathy, often in combination with CaNa₂EDTA. The most common side effect of treatment with dimercaprol is a 50 mmHg rise in systolic and diastolic blood pressure among many others. In the 1960's, dimercaprol was modified into DMSA, which has fewer side effects and thus has fallen out of favor for treatment of lead poisoning.

Chelation therapy with CaNa₂EDTA, DMSA, and dimercaprol all are effective in reducing blood lead levels. However, a rebound in BLL often occurs following treatment, which likely results from absorbed lead emanating from bone and soft tissues. Considering the considerable side effects associated with chelation therapy, it is typically not practiced addressing health problems associated with low level lead poisoning discussed above.

Zeolites, microporous aluminosilicate ion exchangers based on tetrahedral frameworks, have been proposed for treating chronic lead poisoning and are taken in pill form (See U.S. Pat. No. 11,038,748). However, zeolites have limited stability in blood and the acidic environment of the gastrointestinal tract. In acidic solution, Al naturally has octahedral coordination and can be extracted from the tetrahedral zeolite framework. Indeed, treatment with acidic solution is one strategy to modify zeolite composition via dealumination, see U.S. Pat. No. 6,982,074. Also, in U.S. Pat. No. 11,038,748, a particulate sodium aluminosilicate is claimed with 90% of the particulates between a size of 90 and 150 microns (μm). A process is disclosed to achieve this particle size distribution that seems to involve screening using sieves. More recently, examples of microporous ion exchangers that are essentially insoluble in fluids, such as bodily fluids (especially blood), have been developed, including zirconium-based silicates and titanium-based silicates as provided in U.S. Pat. Nos. 5,888,472; 5,891,417; and 6,579,460. The use of these zirconium-based silicate or titanium-based silicate microporous ion exchangers to remove toxic ammonium cations from blood or dialysate is described in U.S. Pat. Nos. 6,814,871; 6,099,737; and 6,332,985. Additionally, it was found that some of these compositions were selective in potassium ion exchange and could remove potassium ions from bodily fluids to treat the disease hyperkalemia, which is discussed in patents U.S. Pat. Nos. 8,802,152; 8,808,750; 8,877,255; 9,457,050; 9,662,352; 9,707,255; 9,844,567; 9,861,658; 10,413,569; 10,398,730; US 2016/0038538; US 2016/0271174; and U.S. Pat. No. 10,695,365. Ex-vivo applications of these materials, for instance in dialysis, are described in U.S. Pat. No. 9,943,637. In particular, U.S. Pat. No. 8,802,152 uses zirconium silicate compositions to treat hyperkalemia via the gastrointestinal tract and identifies particles smaller than 3 microns as undesirable, as these may be absorbed into the patient's bloodstream causing adverse effects, including accumulation in the kidneys. Screening techniques are used to reduce or nearly eliminate particles less than 3 microns from the zirconium silicate product.

Many ion-exchangers have been developed for removal of “heavy metals” from various environments, often waste streams. The heavy metals removed by ion exchangers often include lead, mercury, cadmium, zinc, iron, chromium, copper, cobalt, nickel and even arsenic. Such metals are frequently considered to have the same ion-exchange properties, but, in fact, significant variation exists. For example, a particular zeolite that removes Pb²⁺ from solution does not necessarily remove Hg²⁺ from solution. Indeed, in U.S. Pat. No. 9,233,856 (the '856 patent”), which is incorporated by reference herein in its entirety for all purposes, it is shown that the uptake of Hg²⁺ by zeolites is highly dependent on the framework charge density or equivalently, the Si/Al ratio. High charge density Si/Al=1 zeolites like X (FAU topology) and 4A (LTA topology) are shown to have very low affinity for Hg²⁺ under the test conditions, while Ca²⁺ and Mg²⁺ are easily removed (See US '856 patent at Example 10). In the same test using UZM-9, a zeolite with Si/Al=5.50 that has the same LTA zeolite topology as zeolite 4A, the situation is reversed and there is high selectivity for Hg²⁺, while the selectivity to Ca²⁺ and Mg²⁺ is highly diminished. In the case of zeolite 4A and UZM-9, this result decouples structure from the framework charge density and shows the importance of the latter in ion-exchange selectivity (See '856 patent at Example 11). Meanwhile, zeolite X is shown to be excellent at removing Pb²⁺ from aqueous solution, while it performs poorly at removing Hg²⁺ (See '856 patent at Comparative Example 13). Hence, to determine an ion exchanger's cation selectivity, the relevant data must be collected for each remediated metal cation/resident metal cation/ion exchanger combination.

A process was disclosed for the removal of Sr²⁺ ions from bodily fluids using Zr, Ti, Sn-based metallate ion exchangers, see U.S. Pat. No. 11,577,014 (“the '104 patent”), which is incorporated by reference herein in its entirety for all purposes. The majority of the ion exchangers provided in the '014 patent are crystalline and amorphous metallosilicates, while a metal oxide was also disclosed, a commercial sodium nonatitanate sample. A process has also been disclosed for the removal of Hg² from bodily fluids utilizing Ti metallates, see U.S. Pat. No. 11,484,875, which is incorporated by reference herein in its entirety for all purposes. Ti was required in the compositions with Nb and Si optional. Very specific topology/composition combinations were required for efficient Hg²⁺ removal, including metal silicates Ti—Nb sitinakite and acid treated zorite as well as a metal oxide, a commercial sodium nonatitanate from Allied-Signal. Also, US patent application, U.S. Pat. No. 11,964,266 (“the '266 application), which is incorporated by reference herein in its entirety for all purposes, discloses the use of metallosilicates and metal oxides for the removal of cobalt, lead, cadmium, and chromium ions from bodily fluids using Zr, Ti, Sn-based metallate ion exchangers. The majority of the ion exchangers of the '266 application are crystalline and amorphous metallosilicates while two metal oxides were disclosed, a commercial sample of sodium nonatitanate from Allied-Signal and a commercial potassium octatitanate product from Honeywell. Unlike the sodium nonatitanate, the potassium octatitanate was not effective for efficient Hg²⁺ and Sr²⁺ removal.

The present disclosure provides for, and includes, a process to remove Pb²⁺ ions from bodily fluids, which uses polycrystalline aggregate metal titanate ion exchangers that are essentially insoluble in bodily fluids, especially gastrointestinal fluids. The metal titanate ion exchangers have an empirical formula on an anhydrous basis of:

AₘTiₓM_{y}O_{z}

where A is an exchangeable cation selected from the group consisting of potassium ion, sodium ion, lithium ion, calcium ion, magnesium ion, hydronium ion or mixtures thereof, M is at least one framework metal selected from the group consisting of niobium (5+), zirconium (4+), tin (4+), iron (3+), iron (2+), cobalt (2+), and manganese (2+), “m” is the mole ratio of A to total metal (total metal=Ti+M) and has a value from 0.10 to 0.60, “x” is the mole fraction of total metal that is Ti and has a value from 0.5 to 1, “y” is the mole fraction of total metal that is M and has a value from zero to 0.5, where x+y=1, and “z” is the mole ratio of O to total metal and has a value from 1.55 to about 2.85 and are synthesized in the presence of at least one multihydroxyl-containing complexing agent (MHCA). These compositions can be essentially insoluble in bodily fluids (at neutral and mildly acidic or basic pH), so they may be used to remove Pb²⁺ toxins from gastrointestinal fluid systems and may be suitable for ingestion in vivo.

### SUMMARY OF THE INVENTION

The present disclosure provides for, and includes, a particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85,
wherein the particulate metal titanate ion exchanger having been synthesized in the presence of at least one multihydroxyl-containing complexing agent (MHCA), and wherein the particulate metal titanate ion exchanger exhibits a median particle size of greater than 3 microns (μm).

The present disclosure also provides for, and includes, a macroporous particulate titanate ion exchanger having an empirical formula on an anhydrous basis of:

AₘTiO_{z}
wherein
A is an exchangeable cation selected from the group consisting of potassium ion, hydronium ion, and a mixture thereof; “m” is the mole ratio of A to Ti and has a value from 0.10 to 0.60; and “z” is the mole ratio of O to Ti and has a value from 2.05 to 2.60,
wherein the macroporous titanate ion exchanger having been synthesized in the presence of a multihydroxyl-containing complexing agent (MHCA) that is d-sorbitol, wherein the macroporous particulate titanate ion exchanger exhibits a median particle size that is between 25 to 125 microns (μm), wherein less than 3.0% of the particles of the macroporous particulate titanate ion exchanger have a particle size less than 3 microns (μm), and wherein the macroporous particulate titanate ion exchanger has a Brunauer-Emmett-Teller (BET) surface area of at least 150 square meters per gram (m²/g).

The present disclosure also provides for, and includes, a macroporous particulate titanate ion exchanger having an empirical formula on an anhydrous basis of:

AₘTiO_{z}
wherein
A is an exchangeable cation selected from the group consisting of potassium ion, hydronium ion, and a mixture thereof; “m” is the mole ratio of A to Ti and has a value from 0.10 to 0.60; and “z” is the mole ratio of O to Ti and has a value from 2.05 to 2.60,
wherein the macroporous titanate ion exchanger having been synthesized in the presence of a multihydroxyl-containing complexing agent (MHCA) that is d-sorbitol, wherein the macroporous particulate titanate ion exchanger exhibits a median particle size that is between 25 to 125 microns (μm), wherein less than 0.5% of the particles of the macroporous particulate titanate ion exchanger have a particle size less than 3 microns (μm), and wherein the macroporous particulate titanate ion exchanger has a Brunauer-Emmett-Teller (BET) surface area of at least 150 square meters per gram (m²/g).

The present disclosure also provides for, and includes, a method for manufacturing a tablet or capsule or for oral administration, the tablet or capsule comprising a particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the particulate metal titanate ion exchanger exhibits a median particle size of greater than 3 microns (μm),
the method comprising the steps of:
   (a) forming a reaction mixture comprising reactive sources of A, Ti, at least one multihydroxyl-containing complexing agent (MHCA), optionally M, optionally hydrogen peroxide, optionally a complexing agent (C), and water,
   (b) heating the reaction mixture for a period of time to form a metal titanate ion exchanger,
   (c) treating the synthesized metal titanate ion exchanger via acid extraction and/or ion exchange with alkali metals, alkaline earth metals, or mixtures thereof, to form the particulate metal titanate ion exchanger with the desired composition,
   (d) optionally admix the metal titanate ion exchanger with one or more pharmaceutically acceptable adjuvants, diluents or carriers to form a metal titanate ion exchanger medicament, and
   (e) forming a capsule or tablet comprising the particulate metal titanate ion exchanger
wherein the reaction mixture comprises a composition expressed in terms of mole ratios of the oxides of:

p A₂O: a TiO₂:b MO_{q/2}:c H₂O₂:d MHCA:e C:f H₂O
wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000.

The present disclosure also provides for, and includes, a method for selectively removing Pb²⁺ toxins from gastrointestinal fluid, the process comprising contacting the fluid containing the toxins with a particulate metal titanate ion exchanger, resulting in an ion exchanged ion exchanger and thereby removing the Pb²⁺ toxins from the fluid, the particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the metal titanate ion exchanger having been synthesized in the presence of at least one multihydroxyl-containing complexing agent (MHCA), wherein the particulate metal titanate ion exchanger exhibits a median particle size of greater than 3 microns (μm), and wherein the particulate metal titanate ion exchanger minimally disrupts the levels of any one or more ions selected from Na⁺, Mg²⁺, K⁺, and Ca²⁺.

The present disclosure also provides for, and includes, an intracorporeal process for removing Pb²⁺ toxins from gastrointestinal fluid, the process comprising contacting the fluid containing the toxins with a particulate aggregate metal titanate ion exchanger resulting in an ion exchanged ion exchanger thereby removing the toxins from the fluid, the particulate metal titanate ion exchanger having an empirical formula on an anhydrous basis of:

AₘTiₓM_{y}O_{z}

where A is an exchangeable cation selected from the group consisting of potassium ion, sodium ion, lithium ion, calcium ion, magnesium ion, hydronium ion or mixtures thereof, M is optionally at least one framework metal selected from niobium (5+), zirconium (4+), tin (4+), iron (3+), iron (2+), cobalt (2+), and manganese (2+), “m” is the mole ratio of A to total metal (total metal=Ti+M) and has a value from 0.10 to 0.60, “x” is the mole fraction of total metal that is Ti and has a value from 0.5 to 1, “y” is the mole fraction of total metal that is M and has a value from zero to 0.5, where x+y=1, and “z” is the mole ratio of O to total metal and has a value from 1.55 to 2.85, where the metal titanate ion exchanger having been synthesized in the presence of at least one multihydroxyl-containing complexing agent (MHCA).

The present disclosure also provides for, and includes, a process for preparing a particulate metal titanate ion exchanger, the particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, the process comprising the steps of
   (a) forming a reaction mixture comprising reactive sources of A, Ti, at least one multihydroxyl-containing complexing agent (MHCA), optionally M, optionally hydrogen peroxide, optionally a complexing agent (C), and water, and
   (b) heating the reaction mixture at a temperature of about 85° C. to about 225° C. for a time period of 0.5 to 30 days to form the particulate metal titanate ion exchanger,
wherein the reaction mixture comprises a composition expressed in terms of mole ratios of the oxides of:

p A₂O: a TiO₂:b MO_{q/2}:c H₂O₂:d MHCA:e C:f H₂O
wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000.

The present disclosure also provides for, and includes, the synthesis of a metal titanate ion exchanger composition from highly alkaline aqueous solution phase reaction mixtures, the solution character facilitated by complexing agents that include at least one MHCA, hydrogen peroxide and a carboxylate, such as citric acid. The solution character of the reaction mixture enhances the reactivity of the system enabling metal incorporation into the lattice under conditions where any of the metals (Ti, Sn, Zr, Nb, Co, Fe, Mn) would be precipitated and otherwise not easily transported during synthesis. In an aspect, hydrothermal synthesis of the ion exchange composition from highly alkaline aqueous solution in the presence of MHCAs, especially d-sorbitol, and the other complexing agents promotes the formation of polycrystalline aggregates that include spheres, interpenetrating spheres, slabs, and other morphologies that are usually sufficiently large (>3 μm) that they would not be absorbed in the gastrointestinal tract.

The present disclosure also provides for, and includes, the synthesis of a metal titanate ion exchanger composition from TiO₂ powders in a highly alkaline reaction mixture containing complexing agents, at least one of which is an MHCA, imparting unique properties to the resulting metal titanate ion exchanger. In an aspect of a titanate ion exchange composition, synthesis in the presence of at least one MHCA can provide for the formation of macroporous polycrystalline aggregate particles from the conversion of TiO₂ powder that are large enough to avoid absorption in the gastrointestinal tract. In an aspect of a metal titanate composition, synthesis in the presence of multiple complexing agents that include at least one MHCA and for instance, a carboxylate such as citric acid and optionally hydrogen peroxide, solubilizes the source of the metal substituent M in the highly alkaline solution, making it available for reaction with and incorporation into the TiO₂ powder, and capable of forming large metal titanate macroporous polycrystalline aggregates that are usually large enough to avoid absorption into the gastrointestinal tract.

The present disclosure also provides for, and includes, the synthesis of a metal titanate ion exchanger composition from pre-formed TiO₂, e.g., from spray dried TiO₂ spheres in an alkaline reaction mixture containing complexing agents, at least one of which is an MHCA, imparting unique properties to the resulting metal titanate ion exchanger. In an aspect of a titanate ion exchange composition, synthesis in the presence of at least one MHCA enables the conversion of the preformed TiO₂ spheres into macroporous alkali titanate spheres that are large enough to avoid absorption into the gastrointestinal tract. In the case of a metal titanate composition, synthesis in the presence of multiple complexing agents that include at least one MHCA and for instance a carboxylate such as citric acid and optionally hydrogen peroxide, solubilizes the source of the metal substituent M in the highly alkaline solution, making it available for reaction with and incorporation into the formed TiO₂ spheres, providing the conversion to macroporous metal titanate spheres that are large enough to avoid absorption into the gastrointestinal tract.

The present disclosure also provides for, and includes, forming of metal titanates of the present disclosure, including mixtures of different metal titanates, into a formed product, e.g., spray dried spheres, where each of the metal titanates having been synthesized in the presence of at least one MHCA and the resulting formed product being macroporous spheres are of sufficient size to avoid absorption into the gastrointestinal tract.

The present disclosure also provides for, and includes, a process for removing Pb²⁺ containing toxins from fluids, the process comprising contacting the fluid containing the toxins with a metal titanate ion exchanger resulting in an ion exchanged ion exchanger thereby removing the toxins from the fluid, the metal titanate ion exchanger having an empirical formula on an anhydrous basis of:

AₘTiₓM_{y}O_{z}

where A is an exchangeable cation selected from the group consisting of potassium ion, sodium ion, lithium ion, calcium ion, magnesium ion, hydronium ion or mixtures thereof, M is optionally at least one framework metal selected from niobium (5+), zirconium (4+), tin (4+), iron (3+), iron (2+), cobalt (2+), and manganese (2+), “m” is the mole ratio of A to total metal (total metal=Ti+M) and has a value from 0.10 to 0.60, “x” is the mole fraction of total metal that is Ti and has a value from 0.5 to 1, “y” is the mole fraction of total metal that is M and has a value from zero to 0.5, where x+y=1, “z” is the mole ratio of O to total metal and has a value from 1.55 to 2.85 where the metal titanate ion exchanger having been synthesized in the presence of at least one complexing agent that is a multihydroxyl-containing complexing agent (MHCA).

### BRIEF DESCRIPTION OF THE DRAWINGS

FIG. 1, comprising FIGS. 1A-1F, shows scanning electron microscope images (SEM) of metal titanate products derived from syntheses in aqueous solution. in the presence of the MHCA d-sorbitol, large polycrystalline aggregates of the metal titanate ion exchangers were isolated from syntheses carried out in homogenous solution.

FIG. 1A shows a scanning electron microscope image (SEM) of Na_{0.25}Fe_{0.20}Ti_{0.80}, from Example 5B having polycrystalline sphere morphology.

FIG. 1B shows an SEM image of Na_{0.25}Fe_{0.20} Ti_{0.80}, Example 5B. Polycrystalline slab morphology.

FIG. 1C shows an SEM of Na_{0.33}Fe_{0.38}Ti_{0.62}, Example 7B. Polycrystalline interpenetrating spheres.

FIG. 1D shows an SEM of Na_{0.33}Fe_{0.38}Ti_{0.62}, Example 7B. Polycrystalline interpenetrating spheres.

FIG. 1E shows an SEM of Na_{0.39}Zr_{0.03}Ti_{0.97}, Example 9. Polycrystalline interpenetrating spheres.

FIG. 1F shows an SEM of K—Nb—Ti—O, Example 11. Slabs of polycrystalline interpenetrating spheres.

FIG. 2, including FIGS. 2A-2L, shows that metal titanate ion exchangers consisting of large polycrystalline aggregates are synthesized from TiO₂ powders and preformed spray dried TiO₂ spheres in strongly alkaline media in the presence of multihydroxyl-containing complexing agents.

FIG. 2A shows an SEM of K_{0.26}Ti, Example 17. Polycrystalline sponge-like morphology derived from TiO₂ powder in the presence of KOH/catechol solution.

FIG. 2B shows an SEM of Na—Ti—O, Example 20A. Complex aggregates of very large polycrystalline interpenetrating spheres derived from preformed TiO₂ spheres hydrothermally treated in the presence of NaOH/d-sorbitol solution.

FIG. 2C shows an SEM of Na—Fe—Ti—O, Example 21A. Na—Fe—Ti—O polycrystalline spheres derived from preformed TiO₂ spheres treated hydrothermally with Fe(NO₃)₃/citric acid/d-sorbitol/NaOH solution.

FIG. 2D shows an SEM of Na—Fe—Ti—O, Example 21A. Na—Fe—Ti—O polycrystalline spheres derived from preformed TiO₂ spheres treated hydrothermally with Fe(NO₃)₃/citric acid/d-sorbitol/NaOH solution.

FIG. 2E shows an SEM of K_{0.40}Ti, Example 24A. Spongy macroporous polycrystalline aggregates derived from hydrothermal treatment of TiO₂ powder in the presence of d-sorbitol/KOH solution.

FIG. 2F shows an SEM of K_{0.40}Ti, Example 24A. Close up of the spongy macroporous polycrystalline network in the aggregate. Derived from hydrothermal treatment of TiO₂ powder in the presence of d-sorbitol/KOH solution.

FIG. 2G shows an SEM of K_{0.30}Ti, composite sample, Example 24E. Spongy macroporous polycrystalline aggregates derived from hydrothermal treatment of TiO₂ powder in the presence of d-sorbitol/KOH solution.

FIG. 2H shows an SEM of K_{0.30}Ti, composite sample, Example 24E. Close-up view of the spongy macroporous polycrystalline network in the aggregates. Derived from hydrothermal treatment of TiO₂ powder in the presence of d-sorbitol/KOH solution.

FIG. 2I shows an SEM of K_{0.28}Ti, composite sample, Example 25D. Field view of K_{0.28}Ti spheres derived from hydrothermal treatment of preformed spray dried TiO₂ spheres in the presence of d-sorbitol/KOH solution.

FIG. 2J shows an SEM of K_{0.28}Ti, composite sample, Example 25D. Close-up view of K_{0.28}Ti spheres derived from hydrothermal treatment of preformed spray dried TiO₂ spheres in the presence of d-sorbitol/KOH solution.

FIG. 2K shows an SEM of K_{0.16}Ti, acid-treated composite sample, Example 25E. Field view of acid-treated composite sample of Example 25D. Spheres maintain structural integrity during acid treatment.

FIG. 2L shows an SEM of K_{0.16}Ti, acid-treated composite sample, Example 25E. Close-up view of acid-treated composite sample of Example 25D. Spheres maintain structural integrity during acid treatment.

FIGS. 3-8 show particle size distribution data for selected examples and comparative examples of the prior art. Definition of terms: D3 (μm): The smallest 3 volume percent of particles in the sample is smaller than the particle size D3 (μm), measured in microns. Similarly, D10 (μm) is the particle size under which the smallest 10 volume percent of the sample occurs. D50 (μm) is equivalent to the median particle size, the particle size under which the smallest 50 volume percent of the sample occurs. D90 (μm) is the particle size under which the smallest 90 volume percent of the sample occurs. The parameter <3 μm (vol %), indicates the volume percent of the sample that is less than 3 μm in size. The Mean is the average particle size in the sample.

FIG. 3 shows the particle size distribution of a sodium nonatitanate prior art material from comparative Example C1.

FIG. 4 shows the particle size distribution of potassium octatitanate prior art material from comparative Example C2.

FIG. 5 shows the particle size distribution of Example 24F product prepared from TiO₂ powder treated hydrothermally in the presence of d-sorbitol/KOH solution followed by acid treatment.

FIG. 6 shows the particle size distribution of the Example 25D product prepared from preformed spray dried TiO₂ spheres treated hydrothermally in the presence of KOH/d-sorbitol solution.

FIG. 7 shows the particle size distribution of the Example 25E product prepared from preformed spray dried TiO₂ spheres treated hydrothermally in the presence of KOH/d-sorbitol solution followed by acid treatment.

FIG. 8 shows the particle size distribution for the Example 26 product synthesized from nano-titania powder treated hydrothermally in the presence of KOH/d-sorbitol solution.

### DETAILED DESCRIPTION

### A. Metal Titanate Ion Exchangers

The present disclosure provides for, and includes, new processes for removing Pb²⁺-containing toxins from gastrointestinal fluids. Without being limited by theory, one aspect of the processes of the present disclosure is an ion exchanger which has a large capacity and strong affinity, i.e., selectivity for Pb²⁺ ions, both free and complexed. Again, not limited by an particular theory, another aspect of the processes of the present disclosure is metal titanate ion exchangers being synthesized in the presence of at least one multihydroxyl-containing complexing agent (MHCA), which provides for facile chemistry of titanium and the M elements in highly basic aqueous solution and imparts properties such as favorable particulate size that helps avoid undesirable absorption during treatment in the gastrointestinal tract. The metal titanate compositions of the present disclosure are identified as alkali metal titanate compositions as synthesized, which besides Ti⁴⁺ may additionally contain Mn²⁺, Co²⁺, Fe²⁺, Fe³⁺, Sn⁴⁺, Zr⁴⁺, and Nb⁵⁺, or mixtures thereof and may additionally be modified by ion exchange. They are further identified by their composite empirical formula (on an anhydrous basis) which is:

AₘTiₓM_{y}O_{z}

The composition has a framework structure(s) composed of at least TiO_{6/n} octahedral units where n may be 2 or 3 or both, or optionally may include MO_{6/n} octahedral units where n may be 2 or 3 or both, although other coordination environments may occur for Ti and M. A is an exchangeable cation selected from the group consisting of potassium ion, sodium ion, lithium ion, calcium ion, magnesium ion, hydronium ion or mixtures thereof, M is an optional framework metal selected from the group consisting of cobalt (2+), manganese (2+), iron (2+), iron (3+), tin (4+), zirconium (4+) or niobium (5+) or mixtures thereof, “m” is the mole ratio of A to total metal (total metal=Ti+M) and has a value from 0.10 to 0.6, “x” is the mole fraction of total metal that is Ti and has a value from 0.5 to 1.0, “y” is the mole fraction of total metal that is M and has a value from zero to 0.5, where x+y=1, and “z” is the mole ratio of O to total metal and has a value from 1.55 to 2.85.

In an aspect, the synthesis of metal titanate ion exchangers of the present disclosure utilizes novel chemistry to facilitate incorporation of M and Ti into the same network. In an aspect, metal titanates are cation exchange compositions, which require a negative charge on the metal oxide component. To obtain negatively charged metal oxide frameworks that can operate as ion exchangers, synthesis must occur at higher pHs than those associated with the point of zero charge of the metal oxides, the pH associated with neutral metal oxides. In an aspect, the metal titanate ion exchangers of the present disclosure are synthesized in alkaline solution, usually highly alkaline solution. Ti and the M metals are insoluble in highly alkaline solution, which can be detrimental to successful incorporation of the metals into the same phase. Metals are often available as salts and form acidic solutions on dissolution in water. This is true for the M metals Sn, Zr, Co, Mn, and Fc. The solubility of these metals can be extended to basic pHs using complexing agents such as citric acid, amines or EDTA, perhaps as high as pH 10-12, but these will precipitate when taken to the higher pHs required to synthesize the metal titanates of the present disclosure. Dissolving Ti and Nb reagents in aqueous solutions can also be problematic, as salts such as TiCH₄ or NbCl₅ and alkoxides such as titanium isopropoxide, Ti(OiPr)₄, and niobium ethoxide, Nb(OEt)₅ can hydrolyze and precipitate immediately. However, aqueous solutions of such Ti and Nb reagents can be formed in the presence of sufficient H₂O₂ in acidic solution. The solubility of these Ti and Nb solutions can be extended to higher pH via addition of a complexing agent such as citric acid; the case for Nb-peroxo-citrate solution formation at pH=7.5 from poorly soluble niobium oxalate is documented, see Chem. Mater., vol. 9, pp. 580-587, 1997. Peroxo-citrate solutions of Nb and Ti also precipitate when further treated with hydroxide, and solubility at high pH is not realized. But with the addition of a multihydroxyl-containing complexing agent (MHCA) such as d-sorbitol or catechol that has a high pKₐ to these metal citrate solutions or metal peroxo-citrate solutions, the solutions can retain their solution character as hydroxide is added to increase the pH, resulting in stable solutions of Ti and the M metals at very high pH, greater than pH=14 if desired. Not to be bound by theory, at high pH the hydroxyls of sugar alcohols such as d-sorbitol or aromatic diols such as catechol can be deprotonated, becoming strong complexing agents that can stabilize metals in highly basic aqueous solutions. The availability of Ti and the M metals in these highly basic solutions facilitates their mutual incorporation into the metal titanate ion exchanger compositions of the present disclosure during their synthesis. Hydrothermal digestion of these reaction mixtures forms products consisting of large polycrystalline aggregates, often spheres, interpenetrating spheres, slabs, and other morphologies; the particulates are often large enough (e.g., greater than 3 μm) to avoid absorption into the gastrointestinal tract.

In an aspect, MHCAs can be used to enhance the synthesis of the metal titanate ion exchanger compositions of the present disclosure from TiO₂ powder. Hydrothermal treatment of TiO₂ powders in alkaline solution in the presence of MHCAs facilitates the formation of macroporous polycrystalline aggregates that are larger than those observed in the solution syntheses and, in an aspect, are large enough to avoid absorption into the gastrointestinal tract. M metals are incorporated into the TiO₂ powders by preparing a highly basic solution containing the M metal using the appropriate complexing agents such as H₂O₂, citric acid, and an MHCA to maintain the M metal in solution and help transport Ti, mixing the TiO₂ powder with the solution, and treating the resulting mixture hydrothermally, thereby forming a product comprised of large macroporous polycrystalline aggregates imparted by the presence of the MHCAs.

In an aspect, MHCAs can be applied to pre-formed TiO₂ powder starting materials, specifically spray dried spheres. Hydrothermal treatment of the TiO₂ spheres with alkaline solutions containing MHCAs yields macroporous spheres. The M metals are incorporated into the TiO₂ spheres in a similar manner as the TiO₂ powders, e.g., by preparation of a highly basic solution containing the M metal using the appropriate complexing agents such as H₂O₂, citric acid, and an MHCA, mixing the TiO₂ spheres with the M-containing alkaline solution, and treating the resulting mixture hydrothermally, thereby forming a macroporous sphere. The product spheres are large enough to avoid absorption by the gastrointestinal tract.

The metal titanate ion exchanger compositions of the present disclosure may be prepared by a hydrothermal crystallization of a reaction mixture prepared by combining a reactive source of titanium and optionally one or more M metal, at least one alkali metal, at least one complexing agent including at least one MHCA, optionally hydrogen peroxide and water. Without being limited by theory, an alkali metal can act as a framework charge balancing agent as well as a templating agent. Examples of titanium metal sources include, but are not limited to titanium alkoxides, titanium tetrachloride, titanium trichloride, amorphous titanium oxyhydroxide, titanium dioxide, rutile titanium dioxide, anatase titanium dioxide, nano-sized titanium dioxide (crystallite size of about 200 nm, preferably 100 nm or less), and preformed spray dried TiO₂ spheres. Examples of M metal sources include, but are not limited to cobalt acetate, cobalt nitrate, cobalt chloride, manganese acetate, manganese nitrate, manganese chloride, manganese sulfate, iron (3+) nitrate, iron (3+) chloride, iron (2+) chloride, iron (2+) chloride, iron (2+) sulfate, iron (2+) acetate, tin (4+) chloride, zirconyl chloride, zirconyl nitrate, zirconium alkoxides, zirconium acetate, zirconium chloride, niobium oxalate, ammonium oxo-niobium dioxalate, niobium chloride, and niobium alkoxides. Examples of A alkali sources include, but are not limited to potassium hydroxide, sodium hydroxide, lithium hydroxide, sodium halide, potassium halide, lithium halide, sodium acetate, potassium acetate, and lithium acetate. Hydrogen peroxide is an optional complexing agent for which the preferred source is 30 wt. % aqueous solution. Examples of multihydroxyl-containing complexing agents (MHCAs) include, but are not limited to sugar alcohols such as d-sorbitol, mannitol, and xylitol, sugars such as glucose, and fructose, and multihydroxyl-containing aromatics such as catechol. MHCAs are understood to contain at least two hydroxyl groups. One or more complexing agents (C) may be employed in any reaction. Complexing agent sources include, but are not limited to carboxylates, such as citric acid and tartaric acid, nitrogen-containing complexing agents like EDTA, and bipyridine, and mixtures thereof. In an aspect, the hydrothermal process used to prepare the titanium metallate ion exchange compositions of the present disclosure involves forming a reaction mixture which in terms of molar ratios of the oxides is expressed by the formula:

p A₂O:a TiO₂:b MO_{q/2}:c H₂O₂:d MHCA:e C:f H₂O

where “p” has a value from about 4 to 40, “a” has a value from about 0.5 to 1, “b” has a value from 0 to 0.5, a+b=1, “c” has a value from 0 to 6, “MHCA” is at least one multihydroxyl-containing complexing agent, “d” has a value from 0.2 to 4, “C” is at least one complexing agent, “e” has a value of 0 to 4, and “f” has a value from 20 to 1000. A reaction mixture can be prepared by mixing the desired sources of alkali metal, titanium, multihydroxyl-containing complexing agent MHCA, optionally hydrogen peroxide, optionally complexing agents C, optionally M metal and water to give the desired mixture. Also, the final reaction mixture has a basic pH, and preferably a pH of at least 12.5. The basicity of the mixture is controlled by adding alkali hydroxide. Having formed the reaction mixture, it is next reacted at a temperature of about 85° C. to about 225° C. for a period of about 0.5 to about 30 days in a sealed reaction vessel under autogenous pressure. After the allotted time, the mixture is filtered or centrifuged to isolate the solid product which is washed with deionized water and dried in air. As provided above, the compositions of the present disclosure can have a framework structure composed mostly of octahedral TiO_{6/n} units, and optionally octahedral MO_{6/n} units, n=2 or 3, but other coordination environments may be present. Metal titanate ion exchangers of the present disclosure may exhibit various levels of crystallinity or may be amorphous.

As synthesized, in an aspect the metal titanate ion exchanger compositions of the present disclosure may contain some of the alkali metal templating agent in the pores, between layers and chains, or in other charge balancing positions. These metals are described as exchangeable cations, meaning that they can be exchanged with other (secondary) A′ cations. Generally, the A exchangeable cations can be exchanged with A′ cations selected from other alkali metal cations (K⁺, Na⁺, Li⁺), alkaline earth cations (Mg²⁺, Ca²⁺), hydronium ion, or mixtures thereof. As used herein, an A′ cation is different from an A cation. Methods used to exchange one cation for another are well known in the art and involve contacting metal titanate ion exchanger compositions of the present disclosure with a solution containing the desired cation (at molar excess) at exchange conditions. Exchange conditions include a temperature of about 25° C. to about 100° C. and a time of about 20 minutes to about 2 hours. The particular cation (or mixture thereof) which is present in the final product will depend on the particular use of the composition and the specific composition being used. For instance, for the treatment of a hypocalcemic patient low on Ca²⁺, one specific composition is a Ca²⁺-containing ion exchanger where the A′ cation is a mixture of Na⁺, Ca²⁺ and H⁺ ions.

In an aspect, synthesized powder forms of the metal titanate ion exchangers of the present disclosure are generally particles that are greater than 3 microns in size, which is desirable. However, it may be desirable to have a subset of these particles being smaller than 3 microns with an upper limit of 3% less than 3 microns in size by volume. In an aspect, a metal titanate ion exchanger of the present disclosure exhibits a median particle size greater than 3 microns. In an aspect, a metal titanate ion exchanger of the present disclosure has a median particle size ranging from 25 to 125 microns.

It is also within the scope of the present disclosure that ion exchange compositions can be used in the methods provided herein in an as-synthesized powder form or can be formed into various shapes by means well known in the art. Examples of these various shapes include pills, extrudates, spheres, pellets, and irregularly shaped particles. See, e.g., US 6579460B1 and U.S. Pat. No. 6,814,871B1. In an aspect, the forming process may include the addition of a binding agent, for example, zirconia, that may require annealing, which may occur by calcination in air at temperatures up to 350° C. for 2 to 6 hours. In an aspect, forming may also occur at several scales, for instance at one scale for the synthesis of the metal titanate ion exchanger and at another scale for delivery of the metal titanate ion exchanger to the body. For example, in Examples 20-23 and 25 provided herein, preformed spray dried TiO₂ spheres are used as the starting material for the synthesis of the metal titanate ion exchangers of tens of microns in diameter, which can then be formed into a larger pill for delivery to the body. Conversely, in an aspect, metal titanate ion exchangers of the present disclosure may first be synthesized from solution, and then one or more of these metal titanates can be spray dried to form a product consisting of larger aggregates, such as spherical aggregates, which may later be formed into a pill. In an aspect, formed pills or other shapes of the metal titanate ion exchangers of the present disclosure may be ingested orally and sequester toxins within the gastrointestinal fluid as the ion exchanger passes through the intestines and is finally excreted. In an aspect, it is possible to protect the ion exchangers of the present disclosure from high acid content (such as is found in the stomach) by coating the shaped articles with various coatings which will not dissolve in the stomach but will dissolve in the intestines.

As provided herein, in an aspect, metal titanate ion exchanger compositions of the present disclosure can have particular utility in adsorbing various Pb²⁺-containing toxins, including free Pb²⁺ ions and Pb²⁺ from complexed Pb²⁺ ions, from gastrointestinal fluids. The metal titanate ion exchangers of the present disclosure are not limited to the treatment of gastrointestinal fluids; they may also be used to remove Pb²⁺ from other bodily fluids, such as blood, blood plasma, urine, and dialysate solutions. As used herein and in the claims, while the current focus is gastrointestinal fluids, bodily fluids will include but not be limited to blood, blood plasma, and gastrointestinal fluids. Also, in an aspect, the metal titanate ion exchanger compositions of the present disclosure may be used to remove Pb²⁺ from bodily fluids of any mammalian body, including but not limited to humans, cows, pigs, sheep, monkeys, gorillas, horses, dogs, etc. The instant process is particularly suited for removing toxins from fluids of a human body.

As provided herein, in an aspect, while the metal titanate ion exchanger compositions of the present disclosure may be synthesized with a variety of exchangeable cations (“A”), it may be preferable to exchange the cation with secondary cations (A′) which are more compatible with blood or do not adversely affect the blood. Without being limited by theory, compositional requirements for the ion exchanger will vary with the needs of the patient. For this reason, preferred cations are potassium, sodium, lithium, calcium, hydronium, and magnesium. In an aspect, preferred compositions include those containing potassium ions. In another aspect, preferred compositions include those containing hydronium ions. In another aspect, preferred compositions include those containing a combination of potassium and hydronium ions. The relative amounts of any two cations within a metal titanate ion exchanger can vary considerably and may depend on the composition itself as well as the concentration of the two ions in the blood.

In an aspect, a particulate metal titanate ion exchanger according to the present disclosure is in a solid dosage form. Solid dosage forms used for oral administration may include capsules, tablets, pills, powders, extrudates, spheres, pellets, granules, and irregularly shaped particles. Among these solid dosage forms, an active particulate metal titanate ion exchanger is mixed with at least one conventional inert excipient (or vehicle), such as sodium citrate or dicalcium phosphate, or mixed with any one or more of the following ingredients: (a) a filler or a compatibilizer, such as starch, lactose, sucrose, glucose, mannitol, and silicic acid; (b) a bonding agent, such as hydroxymethyl cellulose, alginate, gelatin, polyvinylpyrrolidone, sucrose, and gum arabic; (c) a moisturizer, such as glycerin; (d) a disintegrant, such as agar, calcium carbonate, potato starch or tapioca starch, alginic acid, some composite silicates, and sodium carbonate; (e) a slow solvent, such as paraffin; (f) an absorbing accelerator, such as quaternary amine compounds; (g) a wetting agent, such as cetyl alcohol and glyceryl monostearate; (h) an adsorbent, such as kaolin; and (i) a lubricant, such as talc, calcium stearate, magnesium stearate, solid polyethylene glycol, sodium dodecyl sulfate, or a mixture thereof. Dosage forms of capsules, tablets, and pills may also contain a buffer agent.

In an aspect, solid dosage forms such as tablets, sugared pills, capsules, pills, and granules may be prepared using coatings and shells, such as enteric coatings and other materials known in the art. The solid dosage forms may contain opacifiers, and moreover, active particulate metal titanate ion exchangers or particulate metal titanate ion exchangers in such compositions may be released in a portion of the digestive tract in a delayed manner. Non-limiting examples of embedding components that can be employed are polymeric materials and waxy materials. The active particulate metal titanate ion exchangers may also be formed into microcapsules with one or more of the above excipients.

In an aspect, a particulate metal titanate ion exchanger according to the present disclosure is in tablet form. In an aspect, a particulate metal titanate ion exchanger according to the present disclosure is in capsule form. In an aspect, a particulate metal titanate ion exchanger in tablet form further comprises a tablet coating. In an aspect, a particulate metal titanate ion exchanger in capsule form further comprises a capsule coating.

In an aspect a particulate metal titanate ion exchanger according to the present disclosure may be admixed with one or more pharmaceutically acceptable adjuvants, diluents or carriers, for example, lactose, saccharose, sorbitol, mannitol; starch, for example, potato starch, corn starch or amylopectin; cellulose derivative; binder, for example, gelatin or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatin, talcum and titanium dioxide.

In an aspect, a sustained-release preparation of a particulate metal titanate ion exchanger of the present disclosure is administered. Examples of sustained-release preparations include semi-permeable matrices of solid hydrophobic polymers containing the particulate metal titanate ion exchanger, where the matrices are in the form of shaped articles, e.g., films or microcapsules. Examples of sustained-release matrices include polyesters, hydrogels, and polylactides.

In an aspect, a particulate metal titanate ion exchanger of the present disclosure is macroporous. In an aspect, the macroporous particulate metal titanate ion exchanger has a pore diameter between about 50 nanometers (nm) and about 500 nm. In an aspect, the macroporous particulate metal titanate ion exchanger has a pore diameter between about 50 nm and about 400 nm. In an aspect, the macroporous particulate metal titanate ion exchanger has a pore diameter between about 50 nm and about 300 nm. In an aspect, the macroporous particulate metal titanate ion exchanger has a pore diameter between about 50 nm and about 200 nm. In an aspect, the macroporous particulate metal titanate ion exchanger has a pore diameter between about 50 nm and about 100 nm. In an aspect, the macroporous particulate metal titanate ion exchanger has a pore diameter between about 50 nm and about 75 nm. In an aspect, the macroporous particulate metal titanate ion exchanger has a pore diameter between about 100 nm and about 500 nm. In an aspect, the macroporous particulate metal titanate ion exchanger has a pore diameter between about 200 nm and about 500 nm. In an aspect, the macroporous particulate metal titanate ion exchanger has a pore diameter between about 300 nm and about 500 nm. In an aspect, the macroporous particulate metal titanate ion exchanger has a pore diameter between about 400 nm and about 500 nm. In an aspect, the macroporous particulate metal titanate ion exchanger has a pore diameter between about 100 nm and about 350 nm. In an aspect, the macroporous particulate metal titanate ion exchanger has a pore diameter between about 200 nm and about 300 nm. In an aspect, the macroporous particulate metal titanate ion exchanger has a pore diameter between about 225 nm and about 275 nm. In an aspect, the macroporous particulate metal titanate ion exchanger has a pore diameter between about 240 nm and about 260 nm. In an aspect, the macroporous particulate metal titanate ion exchanger has a pore diameter between about 350 nm and about 450 nm.

In an aspect, a particulate metal titanate ion exchanger of the present disclosure has a median particle size in the range of 25 μm to 125 μm. In an aspect, the particulate metal titanate ion exchanger has a median particle size in the range of 25 μm to 100 μm. In an aspect, the particulate metal titanate ion exchanger has a median particle size in the range of 25 μm to 75 μm. In an aspect, the particulate metal titanate ion exchanger has a median particle size in the range of 25 μm to 50 μm. In an aspect, the particulate metal titanate ion exchanger has a median particle size in the range of 25 μm to 35 μm. In an aspect, the particulate metal titanate ion exchanger has a median particle size in the range of 35 μm to 100 μm. In an aspect, the particulate metal titanate ion exchanger has a median particle size in the range of 50 μm to 75 μm. In an aspect, the particulate metal titanate ion exchanger has a median particle size in the range of 35 μm to 125 μm. In an aspect, the particulate metal titanate ion exchanger has a median particle size in the range of 50 μm to 125 μm. In an aspect, the particulate metal titanate ion exchanger has a median particle size in the range of 75 μm to 125 μm. In an aspect, the particulate metal titanate ion exchanger has a median particle size in the range of 100 μm to 125 μm.

In an aspect, a particulate metal titanate ion exchanger of the present disclosure has a particle size distribution d₁₀ value of between about 5 microns (μm) and about 70 μm. In an aspect, the particulate metal titanate ion exchanger has a particle size distribution d₁₀ value of between about 5 μm and about 60 μm. In an aspect, the particulate metal titanate ion exchanger has a particle size distribution d₁₀ value of between about 5 μm and about 50 μm. In an aspect, the particulate metal titanate ion exchanger has a particle size distribution d₁₀ value of between about 5 μm and about 40 μm. In an aspect, the particulate metal titanate ion exchanger has a particle size distribution d₁₀ value of between about 5 μm and about 30 μm. In an aspect, the particulate metal titanate ion exchanger has a particle size distribution d₁₀ value of between about 5 μm and about 20 μm. In an aspect, the particulate metal titanate ion exchanger has a particle size distribution d₁₀ value of between about 20 μm and about 70 μm. In an aspect, the particulate metal titanate ion exchanger has a particle size distribution d₁₀ value of between about 30 μm and about 70 μm. In an aspect, the particulate metal titanate ion exchanger has a particle size distribution d₁₀ value of between about 40 μm and about 70 μm. In an aspect, the particulate metal titanate ion exchanger has a particle size distribution d₁₀ value of between about 50 μm and about 70 μm In an aspect, the particulate metal titanate ion exchanger has a particle size distribution d₁₀ value of between about 10 μm and about 35 μm. In an aspect, the particulate metal titanate ion exchanger has a particle size distribution d₁₀ value of between about 10 μm and about 25 μm. In an aspect, the particulate metal titanate ion exchanger has a particle size distribution d₁₀ value of between about 15 μm and about 45 μm. In an aspect, the particulate metal titanate ion exchanger has a particle size distribution d₁₀ value of between about 25 μm and about 45 μm. In an aspect, the particulate metal titanate ion exchanger has a particle size distribution d₁₀ value of between about 35 μm and about 45 μm. In an aspect, the particulate metal titanate ion exchanger has a particle size distribution d₁₀ value of between about 10 μm and about 20 μm. In an aspect, the particulate metal titanate ion exchanger has a particle size distribution d₁₀ value of between about 12 μm and about 18 μm. In an aspect, the particulate metal titanate ion exchanger has a particle size distribution d₁₀ value of about 15 μm. In an aspect, the particulate metal titanate ion exchanger has a particle size distribution d₁₀ value of between about 40 μm and about 50 μm. In an aspect, the particulate metal titanate ion exchanger has a particle size distribution d₁₀ value of between about 42 μm and about 48 μm. In an aspect, the particulate metal titanate ion exchanger has a particle size distribution d₁₀ value of about 45 μm. In an aspect, the particulate metal titanate ion exchanger has a particle size distribution d₁₀ value of between about 50 μm and about 60 μm. In an aspect, the particulate metal titanate ion exchanger has a particle size distribution d₁₀ value of between about 51 μm and about 57 μm. In an aspect, the particulate metal titanate ion exchanger has a particle size distribution d₁₀ value of about 54 μm.

In an aspect, a particulate metal titanate ion exchanger of the present disclosure has a particle size distribution d₅₀ value of between about 25 microns (μm) and about 125 μm. In an aspect, the particulate metal titanate ion exchanger has a particle size distribution d₅₀ value of between about 25 μm and about 90 μm. In an aspect, the particulate metal titanate ion exchanger has a particle size distribution d₅₀ value of between about 25 μm and about 80 μm. In an aspect, the particulate metal titanate ion exchanger has a particle size distribution d₅₀ value of between about 25 μm and about 70 μm. In an aspect, the particulate metal titanate ion exchanger has a particle size distribution d₅₀ value of between about 25 μm and about 60 μm. In an aspect, the particulate metal titanate ion exchanger has a particle size distribution d₅₀ value of between about 60 μm and about 125 μm. In an aspect, the particulate metal titanate ion exchanger has a particle size distribution d₅₀ value of between about 70 μm and about 125 μm. In an aspect, the particulate metal titanate ion exchanger has a particle size distribution d₅₀ value of between about 80 μm and about 125 μm. In an aspect, the particulate metal titanate ion exchanger has a particle size distribution d₅₀ value of between about 90 μm and about 125 μm. In an aspect, the particulate metal titanate ion exchanger has a particle size distribution d₅₀ value of between about 60 μm and about 90 μm. In an aspect, the particulate metal titanate ion exchanger has a particle size distribution d₅₀ value of between about 60 μm and about 80 μm. In an aspect, the particulate metal titanate ion exchanger has a particle size distribution d₅₀ value of between about 50 μm and about 80 μm. In an aspect, the particulate metal titanate ion exchanger has a particle size distribution d₅₀ value of between about 50 μm and about 70 μm. In an aspect, the particulate metal titanate ion exchanger has a particle size distribution d₅₀ value of between about 45 μm and about 55 μm. In an aspect, the particulate metal titanate ion exchanger has a particle size distribution d₅₀ value of between about 48 μm and about 54 μm. In an aspect, the particulate metal titanate ion exchanger has a particle size distribution d₅₀ value of about 51 μm. In an aspect, the particulate metal titanate ion exchanger has a particle size distribution d₅₀ value of between about 70 μm and about 80 μm. In an aspect, the particulate metal titanate ion exchanger has a particle size distribution d₅₀ value of between about 71 μm and about 77 μm. In an aspect, the particulate metal titanate ion exchanger has a particle size distribution d₅₀ value of about 74 μm. In an aspect, the particulate metal titanate ion exchanger has a particle size distribution d₅₀ value of between about 85 μm and about 95 μm. In an aspect, the particulate metal titanate ion exchanger has a particle size distribution d₅₀ value of between about 89 μm and about 95 μm. In an aspect, the particulate metal titanate ion exchanger has a particle size distribution d₅₀ value of about 92 μm.

In an aspect, a particulate metal titanate ion exchanger of the present disclosure has a particle size distribution d₉₀ value of between about 55 microns (μm) and about 185 μm. In an aspect, the particulate metal titanate ion exchanger has a particle size distribution d₉₀ value of between about 55 μm and about 165 μm. In an aspect, the particulate metal titanate ion exchanger has a particle size distribution d₉₀ value of between about 55 μm and about 145 μm. In an aspect, the particulate metal titanate ion exchanger has a particle size distribution d₉₀ value of between about 55 μm and about 135 μm. In an aspect, the particulate metal titanate ion exchanger has a particle size distribution d₉₀ value of between about 55 μm and about 125 μm. In an aspect, the particulate metal titanate ion exchanger has a particle size distribution d₉₀ value of between about 55 μm and about 115 μm. In an aspect, the particulate metal titanate ion exchanger has a particle size distribution d₉₀ value of between about 55 μm and about 105 μm. In an aspect, the particulate metal titanate ion exchanger has a particle size distribution d₉₀ value of between about 55 μm and about 95 μm. In an aspect, the particulate metal titanate ion exchanger has a particle size distribution d₉₀ value of between about 55 μm and about 85 μm. In an aspect, the particulate metal titanate ion exchanger has a particle size distribution d₉₀ value of between about 85 μm and about 185 μm. In an aspect, the particulate metal titanate ion exchanger has a particle size distribution d₉₀ value of between about 105 μm and about 185 μm. In an aspect, the particulate metal titanate ion exchanger has a particle size distribution d₉₀ value of between about 125 μm and about 185 μm. In an aspect, the particulate metal titanate ion exchanger has a particle size distribution d₉₀ value of between about 145 μm and about 185 μm. In an aspect, the particulate metal titanate ion exchanger has a particle size distribution d₉₀ value of between about 165 μm and about 185 μm. In an aspect, the particulate metal titanate ion exchanger has a particle size distribution d₉₀ value of between about 75 μm and about 145 μm. In an aspect, the particulate metal titanate ion exchanger has a particle size distribution d₉₀ value of between about 85 μm and about 145 μm. In an aspect, the particulate metal titanate ion exchanger has a particle size distribution d₉₀ value of between about 95 μm and about 145 μm. In an aspect, the particulate metal titanate ion exchanger has a particle size distribution d₉₀ value of between about 105 μm and about 145 μm. In an aspect, the particulate metal titanate ion exchanger has a particle size distribution d₉₀ value of between about 115 μm and about 145 μm. In an aspect, the particulate metal titanate ion exchanger has a particle size distribution d₉₀ value of between about 125 μm and about 145 μm. In an aspect, the particulate metal titanate ion exchanger has a particle size distribution d₉₀ value of between about 95 μm and about 105 μm. In an aspect, the particulate metal titanate ion exchanger has a particle size distribution d₉₀ value of between about 99 μm and about 105 μm. In an aspect, the particulate metal titanate ion exchanger has a particle size distribution d₉₀ value of about 102 μm. In an aspect, the particulate metal titanate ion exchanger has a particle size distribution d₉₀ value of between about 135 μm and about 145 μm. In an aspect, the particulate metal titanate ion exchanger has a particle size distribution d₉₀ value of between about 137 μm and about 143 μm. In an aspect, the particulate metal titanate ion exchanger has a particle size distribution d₉₀ value of about 140 μm. In an aspect, the particulate metal titanate ion exchanger has a particle size distribution d₉₀ value of between about 155 μm and about 165 μm. In an aspect, the particulate metal titanate ion exchanger has a particle size distribution d₉₀ value of between about 156 μm and about 162 μm. In an aspect, the particulate metal titanate ion exchanger has a particle size distribution d₉₀ value of about 159 μm.

In an aspect, less than 5% of the particles of a particulate metal titanate ion exchanger of the present disclosure have a particle size of less than 3 μm. In an aspect, less than 4% of the particles of the particulate metal titanate ion exchanger have a particle size of less than 3 μm. In an aspect, less than 3% of the particles of the particulate metal titanate ion exchanger have a particle size of less than 3 μm. In an aspect, less than 2% of the particles of the particulate metal titanate ion exchanger have a particle size of less than 3 μm. In an aspect, less than 1% of the particles of the particulate metal titanate ion exchanger have a particle size of less than 3 μm. In an aspect, less than 0.5% of the particles of the particulate metal titanate ion exchanger have a particle size of less than 3 μm. In an aspect, less than 0.1% of the particles of the particulate metal titanate ion exchanger have a particle size of less than 3 μm. In an aspect, between about 5% and about 0.1% of the particles of the particulate metal titanate ion exchanger have a particle size of less than 3 μm. In an aspect, between about 5% and about 0.5% of the particles of the particulate metal titanate ion exchanger have a particle size of less than 3 μm. In an aspect, between about 5% and about 1% of the particles of the particulate metal titanate ion exchanger have a particle size of less than 3 μm. In an aspect, between about 5% and about 3% of the particles of the particulate metal titanate ion exchanger have a particle size of less than 3 μm. In an aspect, between about 3% and about 0.1% of the particles of the particulate metal titanate ion exchanger have a particle size of less than 3 μm. In an aspect, between about 1% and about 0.1% of the particles of the particulate metal titanate ion exchanger have a particle size of less than 3 μm. In an aspect, between about 0.5% and about 0.1% of the particles of the particulate metal titanate ion exchanger have a particle size of less than 3 μm.

In an aspect, the metal titanate ion exchanger compositions used in the methods provided herein can be synthesized directly and/or meshed to achieve a particle size distribution (PSD) that meets the FDA standard for non-systemic solid particles (greater than 97% of the material by volume has a particle size greater than 3 micrometers). This avoids the particles perfusing out of the small intestine into the blood stream, which can result in the material accumulating in the liver and kidneys. In an aspect, non-systemic metal titanate ion exchanger compositions of the present disclosure can be designed to achieve minimal side effects above that of a placebo. As described in U.S. Pat. Nos. 8,802,152, 8,808,750, 9,844,567, and 10,335,432, and without being limited by any theory, it has been theorized that small particles, less than 3 μm in diameter, of an insoluble powder could potentially be absorbed into a patient's bloodstream through the small intestine resulting in undesirable effects such as the accumulation of particles in the urinary tract of the patient, and particularly in the patient's kidneys. Indeed, the Label for LOKELMA® (sodium zirconium cyclosilicate), an FDA approved drug, describes it as a non-absorbed powder that includes no more than 3% of particles with a diameter below 3 μm. An in vivo pharmacokinetic study in rats showed that a dose of sodium zirconium cyclosilicate powder that meets this specification for particle size distribution was recovered in the feces with no evidence of substantial systemic absorption.

In an aspect, a particulate metal titanate ion exchanger of the present disclosure has a Brunauer-Emmett-Teller (BET) surface area of greater than 150 square meters per gram (m²/g). In an aspect, the particulate metal titanate ion exchanger has a BET surface area of greater than 160 m²/g. In an aspect, the particulate metal titanate ion exchanger has a BET surface area of greater than 170 m²/g. In an aspect, the particulate metal titanate ion exchanger has a BET surface area of greater than 180 m²/g. In an aspect, the particulate metal titanate ion exchanger has a BET surface area of greater than 190 m²/g. In an aspect, the particulate metal titanate ion exchanger has a BET surface area of greater than 200 m²/g. In an aspect, the particulate metal titanate ion exchanger has a BET surface area of greater than 210 m²/g. In an aspect, the particulate metal titanate ion exchanger has a BET surface area of greater than 220 m²/g. In an aspect, the particulate metal titanate ion exchanger has a BET surface area of greater than 230 m²/g. In an aspect, the particulate metal titanate ion exchanger has a BET surface area of greater than 240 m²/g. In an aspect, the particulate metal titanate ion exchanger has a BET surface area of between about 190 m²/g and about 240 m²/g. In an aspect, the particulate metal titanate ion exchanger has a BET surface area of between about 190 m²/g and about 225 m²/g. In an aspect, the particulate metal titanate ion exchanger has a BET surface area of between about 190 m²/g and about 210 m²/g. In an aspect, the particulate metal titanate ion exchanger has a BET surface area of between about 190 m²/g and 200 about m²/g. In an aspect, the particulate metal titanate ion exchanger has a BET surface area of between 200 m²/g and 240 m²/g. In an aspect, the particulate metal titanate ion exchanger has a BET surface area of between about 210 m²/g and about 240 m²/g. In an aspect, the particulate metal titanate ion exchanger has a BET surface area of between about 225 m²/g and about 240 m²/g. In an aspect, the particulate metal titanate ion exchanger has a BET surface area of about 236 m²/g. In an aspect, the particulate metal titanate ion exchanger has a BET surface area of about 203 m²/g. In an aspect, the particulate metal titanate ion exchanger has a BET surface area of about 197 m²/g.

In an aspect, a particulate metal titanate ion exchanger of the present disclosure has a distribution coefficient (K_{d}) for Pb²⁺ of between about 100,000 milliliters per gram (mL/g) and about 2,500,000 mL/g. In an aspect, the particulate metal titanate ion exchanger has a K_{d} for Pb²⁺ of between about 200,000 mL/g and about 2,000,000 mL/g. In an aspect, the particulate metal titanate ion exchanger has a K_{d} for Pb²⁺ of between about 395,000 mL/g and about 1,600,000 mL/g. In an aspect, the particulate metal titanate ion exchanger has a K_{d} for Pb²⁺ of between about 320,000 mL/g and about 1,100,000 mL/g. In an aspect, the particulate metal titanate ion exchanger has a K_{d} for Pb²⁺ of between about 320,000 mL/g and about 1,000,000 mL/g. In an aspect, the particulate metal titanate ion exchanger has a K_{d} for Pb²⁺ of between about 320,000 mL/g and about 900,000 mL/g. In an aspect, the particulate metal titanate ion exchanger has a K_{d} for Pb²⁺ of between about 320,000 mL/g and about 800,000 mL/g. In an aspect, the particulate metal titanate ion exchanger has a K_{d} for Pb²⁺ of between about 320,000 mL/g and about 700,000 mL/g. In an aspect, the particulate metal titanate ion exchanger has a K_{d} for Pb²⁺ of between about 320,000 mL/g and about 600,000 mL/g. In an aspect, the particulate metal titanate ion exchanger has a K_{d} for Pb²⁺ of between about 320,000 mL/g and about 500,000 mL/g. In an aspect, the particulate metal titanate ion exchanger has a K_{d} for Pb²⁺ of between about 320,000 mL/g and about 400,000 mL/g. In an aspect, the particulate metal titanate ion exchanger has a K_{d} for Pb²⁺ of between about 400,000 mL/g and about 1,100,000 mL/g. In an aspect, the particulate metal titanate ion exchanger has a K_{d} for Pb²⁺ of between about 500,000 mL/g and about 1,100,000 mL/g. In an aspect, the particulate metal titanate ion exchanger has a K_{d} for Pb²⁺ of between about 600,000 mL/g and about 1,100,000 mL/g. In an aspect, the particulate metal titanate ion exchanger has a K_{d} for Pb²⁺ of between about 700,000 mL/g and about 1,100,000 mL/g. In an aspect, the particulate metal titanate ion exchanger has a K_{d} for Pb²⁺ of between about 800,000 mL/g and about 1,100,000 mL/g. In an aspect, the particulate metal titanate ion exchanger has a K_{d} for Pb²⁺ of between about 900,000 mL/g and about 1,100,000 mL/g. In an aspect, the particulate metal titanate ion exchanger has a K_{d} for Pb²⁺ of between about 1,000,000 mL/g and about 1,100,000 mL/g. In an aspect, the particulate metal titanate ion exchanger has a K_{d} for Pb²⁺ of between about 400,000 mL/g and about 1,000,000 mL/g. In an aspect, the particulate metal titanate ion exchanger has a K_{d} for Pb²⁺ of between about 500,000 mL/g and about 900,000 mL/g. In an aspect, the particulate metal titanate ion exchanger has a K_{d} for Pb²⁺ of between about 600,000 mL/g and about 800,000 mL/g. In an aspect, the particulate metal titanate ion exchanger has a K_{d} for Pb²⁺ of between about 800,000 mL/g and about 850,000 mL/g. In an aspect, the particulate metal titanate ion exchanger has a K_{d} for Pb²⁺ of between about 950,000 mL/g and about 1,000,000 mL/g. In an aspect, the particulate metal titanate ion exchanger has a K_{d} for Pb²⁺ of about 321,900 mL/g. In an aspect, the particulate metal titanate ion exchanger has a K_{d} for Pb²⁺ of about 495,800 mL/g. In an aspect, the particulate metal titanate ion exchanger has a K_{d} for Pb²⁺ of about 809,500 mL/g. In an aspect, the particulate metal titanate ion exchanger has a K_{d} for Pb²⁺ of about 967,800 mL/g.

In an aspect of the methods provided herein, normal physiological levels of any one or more ions selected from Na⁺, Mg²⁺, K⁺, and Ca²⁺ are minimally disrupted in the subject in need thereof after the administration. In an aspect of the methods provided herein, the physiological levels of the one or more ions are measured in the blood of the subject in need thereof. In an aspect of the methods provided herein, normal physiological levels cations Na⁺, K⁺, Mg²⁺, and Ca²⁺ have unique concentrations in the body (blood) and unique ranges of concentrations in the body that are considered normal. Normal ranges for these cations are given in the table below. In an aspect of the methods provided herein, minimal disruption of the concentrations of these ions would be considered a change of less than about half the magnitude of the variation seen within the normal range. For instance, the normal Na⁺ concentration ranges from 310 to about 333 mg/dL, a variation of 23 mg/dL; a minimal disruption in Na⁺ concentration would in this case be less than ±12 mg/dL. Minimal disruption values are given in the table below.

| | Normal Concentration | |
|---|---|---|
| Cation | Ranges in Blood | Minimal Disruption |
| Na⁺ | 310-333 mg/dL | <±12 mg/dL |
| K⁺ | 14-20 mg/dL | <±3.0 mg/dL |
| Mg²⁺ | 1.5-2.6 mg/dL | <±0.6 mg/dL |
| Ca²⁺ | 8.5-10.5 mg/dL | <±1.0 mg/dL |

In an aspect, a particulate metal titanate ion exchanger of the present disclosure is an acid-treated particulate metal titanate ion exchanger. In an aspect, particulate metal titanate ion exchanger of the present disclosure is a macroporous, acid-treated particulate metal titanate ion exchanger. In an aspect, particulate metal titanate ion exchanger of the present disclosure is a polycrystalline aggregate metal titanate ion exchanger. In an aspect, particulate metal titanate ion exchanger of the present disclosure is a macroporous, polycrystalline aggregate metal titanate ion exchanger. In an aspect, particulate metal titanate ion exchanger of the present disclosure is a particulate metal titanate ion exchanger having spherical morphology or amorphous morphology. In an aspect, particulate metal titanate ion exchanger of the present disclosure is a particulate metal titanate ion exchanger having spherical morphology. In an aspect, particulate metal titanate ion exchanger of the present disclosure is a particulate metal titanate ion exchanger having amorphous morphology.

In an aspect, a particulate metal titanate ion exchanger of the present disclosure is a particulate metal titanate ion exchanger that is in powder form. In an aspect, a particulate metal titanate ion exchanger of the present disclosure is a powder. In an aspect, a particulate metal titanate ion exchanger of the present disclosure is in tablet form. In an aspect, a particulate metal titanate ion exchanger of the present disclosure is in capsule form. In an aspect, a particulate metal titanate ion exchanger of the present disclosure in tablet form further comprises a tablet coating.

In an aspect, a particulate metal titanate ion exchanger of the present disclosure is stable in a liquid environment at a pH of 1-2. In an aspect, a particulate metal titanate ion exchanger of the present disclosure is substantially insoluble at a pH range of 1-7. In an aspect, a particulate metal titanate ion exchanger of the present disclosure is substantially insoluble at a pH range of 7-13. In an aspect, a particulate metal titanate ion exchanger of the present disclosure is substantially insoluble at a pH range of 1-13. In an aspect, a particulate metal titanate ion exchanger of the present disclosure is insoluble at physiological pH. In an aspect, a particulate metal titanate ion exchanger of the present disclosure is substantially insoluble at physiological pH. As used herein, “physiological pH” refers to a pH range of 7.35-7.45. In an aspect, a particulate metal titanate ion exchanger of the present disclosure is insoluble at stomach pH. In an aspect, a particulate metal titanate ion exchanger of the present disclosure is substantially insoluble at stomach pH. As used herein, “stomach pH” refers to a pH range of 1-5.0. As used herein, “substantially insoluble” refers to a solubility of <1% in a fluid. According to the 2015 CRC Handbook, a material is considered soluble in a solvent if a saturated solution contains more than 1% (m/v); any material in which 1 percent or less is dissolved is considered substantially insoluble. The solubility of a compound in body fluids will be quite different than its solubility in pure water because of the effect of proteins, pH, and other solutes in body fluids. When determining solubility of a material in gastrointestinal fluids and the bloodstream the pH of the biological fluids must be considered. Gastrointestinal body fluids vary considerably in their pH (See J. Pharm. Sci., vol. 104, no. 9, pp. 2855-2863, 2015 and J. Indian Soc. Periodontol., vol. 17. No. 4, pp. 461-465, 2013). For example, the pH of the saliva is approximately neutral (mean values ranging from 6.2-7.6), stomach acid has an acidic pH (mean values ranging from 1.7-4.7), and the pH in the small intestine and colon is approximately neutral (mean values ranging from 5-8). Blood is approximately neutral, with a mean pH of about 7.4 (see Crit. Care, vol. 4, pp. 6-14, 2000). Therefore, a material is substantially insoluble in gastrointestinal bodily fluids if 1 percent or less dissolves in a simulated biological fluid across the pH range from 1.5-8. (CRC Handbook of Chemistry and Physics, 95th Ed, CRC Press: Boca Raton, Fl, 2015, W. M. Haynes, Editor in Chief). In an aspect, a particulate metal titanate ion exchanger of the present disclosure is insoluble in one or more bodily fluids selected from the group consisting of blood, urine, and gastrointestinal fluid. In an aspect, a particulate metal titanate ion exchanger of the present disclosure is insoluble in blood. In an aspect, a particulate metal titanate ion exchanger of the present disclosure is insoluble in urine. In an aspect, a particulate metal titanate ion exchanger of the present disclosure is insoluble in gastrointestinal fluid. In an aspect, a particulate metal titanate ion exchanger of the present disclosure is insoluble in both blood and urine. In an aspect, a particulate metal titanate ion exchanger of the present disclosure is insoluble in both blood and gastrointestinal fluid. In an aspect, a particulate metal titanate ion exchanger of the present disclosure is insoluble in both urine and gastrointestinal fluid. In an aspect, a particulate metal titanate ion exchanger of the present disclosure is insoluble in each of blood, urine, and gastrointestinal fluid.

In an aspect, “A” of “AₘTiₓM_{y}O_{z}” is an exchangeable cation. In an aspect, “A” is optionally selected from the group consisting of potassium ion, sodium ion, calcium ion, and hydronium ion. In an aspect, “A” is two or more selected from the group consisting of potassium ion, sodium ion, calcium ion, and hydronium ion. In an aspect, “A” is three or more selected from the group consisting of potassium ion, sodium ion, calcium ion, and hydronium ion. In an aspect, “A” is a potassium ion. In an aspect, “A” is a sodium ion. In an aspect, “A” is a calcium ion. In an aspect, “A” is a hydronium ion.

In an aspect, “M” of “AₘTiₓM_{y}O_{z}” is a framework metal. In an aspect, “M,” a framework metal, is optionally selected from the group consisting of Nb⁵⁺, Zr⁴⁺, Sn⁴⁺, Fe³⁺, Fe²⁺, Co²⁺, Mn²⁺. In an aspect, “M,” a framework metal, is optionally selected from two or more of the group consisting of Nb⁵⁺, Zr⁴⁺, Sn⁴⁺, Fe³⁺, Fe²⁺, Co²⁺, Mn²⁺. In an aspect, “M,” a framework metal, is optionally selected from three or more of the group consisting of Nb⁵⁺, Zr⁴⁺, Sn⁴⁺, Fe³⁺, Fe²⁺, Co²⁺, Mn²⁺. In an aspect, “M” is Nb⁵⁺. In an aspect, “M” is Zr⁴⁺. In an aspect, “M” is Sn⁴⁺. In an aspect, “M” is Fe³⁺. In an aspect, “M” is Fe²⁺. In an aspect, “M” is Co²⁺. In an aspect, “M” is Mn²⁺.

In an aspect, “m” of “AₘTiₓM_{y}O_{z}” is the mole ratio of A to total metal (total metal=Ti+M). In an aspect, “m” has a value between about 0.10 to about 0.60. In an aspect, “m” has a value between about 0.10 to about 0.50. In an aspect, “m” has a value between about 0.10 to about 0.40. In an aspect, “m” has a value between about 0.10 to about 0.30. In an aspect, “m” has a value between about 0.10 to about 0.20. In an aspect, “m” has a value between about 0.20 to about 0.50. In an aspect, “m” has a value between about 0.30 to about 0.50. In an aspect, “m” has a value between about 0.40 to about 0.50. In an aspect, “m” has a value of about 0.28. In an aspect, “m” has a value of about 0.30. In an aspect, “m” has a value of about 0.40.

In an aspect, “x” of “AₘTiₓM_{y}O_{z}” is the mole fraction of total metal that is titanium (Ti). In an aspect, “x” has a value between 0.50 to 1. In an aspect, “x” has a value between 0.60 to 1. In an aspect, “x” has a value between 0.70 to 1. In an aspect, “x” has a value between 0.80 to 1. In an aspect, “x” has a value between 0.90 to 1. In an aspect, “x” has a value between 0.50 to 0.9.0 In an aspect, “x” has a value 0.50 to 0.60. In an aspect, “x” has a value between 0.50 to 0.70. In an aspect, “x” has a value of 0.50. In an aspect, “x” has a value of 0.60. In an aspect, “x” has a value of 0.70. In an aspect, “x” has a value of 0.80. In an aspect, “x” has a value of 0.90. In an aspect, “x” has a value of 1.

In an aspect, “y” of “AₘTiₓM_{y}O_{z}” is the mole fraction of total metal that is “M”. In an aspect, “y” corresponds with “x” according to the stoichiometric equation “x+y=1”. In an aspect, “y” has a value between zero to 0.50. In an aspect, “y” has a value between zero to 0.40. In an aspect, “y” has a value between zero to 0.30. In an aspect, “y” has a value between zero to 0.20. In an aspect, “y” has a value between zero to 0.10. In an aspect, “y” has a value between 0.10 to 0.50. In an aspect, “y” has a value between 0.20 to 0.50. In an aspect, “y” has a value between 0.30 to 0.50. In an aspect, “y” has a value between 0.40 to 0.50. In an aspect, “y” has a value of zero. In an aspect, “y” has a value of 0.10. In an aspect, “y” has a value of 0.20. In an aspect, “y” has a value of 0.30. In an aspect, “y” has a value of 0.40. In an aspect, “y” has a value of 0.50.

In an aspect, “x” is 1 and “y” is 0.

In an aspect, “z” of “AₘTiₓM_{y}O_{z}” is the mole ratio of oxygen (O) to total metal (total metal=Ti+M). In an aspect, “z” has a value between about 1.55 to about 2.85. In an aspect, “z” has a value between about 1.55 to about 2.70. In an aspect, “z” has a value between about 1.55 to about 2.55. In an aspect, “z” has a value between about 1.55 to about 2.40. In an aspect, “z” has a value between about 1.55 to about 2.25. In an aspect, “z” has a value between about 1.55 to about 2.10. In an aspect, “z” has a value between about 1.55 to about 1.95. In an aspect, “z” has a value between about 1.55 to about 1.80. In an aspect, “z” has a value between about 1.55 to about 1.65. In an aspect, “z” has a value between about 1.65 to about 2.85. In an aspect, “z” has a value between about 1.80 to about 2.85. In an aspect, “z” has a value between about 1.95 to about 2.85. In an aspect, “z” has a value between about 2.10 to about 2.85. In an aspect, “z” has a value between about 2.25 to about 2.85. In an aspect, “z” has a value between about 2.40 to about 2.85. In an aspect, “z” has a value between about 2.55 to about 2.85. In an aspect, “z” has a value between about 2.70 to about 2.85. In an aspect, “z” has a value of about 1.55. In an aspect, “z” has a value of about 1.65. In an aspect, “z” has a value of about 1.80. In an aspect, “z” has a value of about 1.95. In an aspect, “z” has a value of about 2.10. In an aspect, “z” has a value of about 2.25. In an aspect, “z” has a value of about 2.40. In an aspect, “z” has a value of about 2.55. In an aspect, “z” has a value of about 2.70. In an aspect, “z” has a value of about 2.85.

In an aspect, Ti of “AₘTiₓM_{y}O_{z}” comprises Ti sourced from one or more Ti-containing compound, including but not limited to, Ti(OiPr)₄, TiCl₄, and TiO₂.

In an aspect, a multihydroxyl-containing complexing agent (MHCA) is a reaction complexing agent that contains at least two hydroxyl groups. In an aspect, MHCAs include but are not limited to sugar alcohols, such as d-sorbitol, mannitol, and xylitol, sugars such as glucose, and fructose, and multihydroxyl-containing aromatics such as catechol. In an aspect, the MHCA is d-sorbitol. In an aspect, the MHCA is mannitol. In an aspect, the MHCA is xylitol. In an aspect, the MHCA is catechol. In an aspect, the MHCA is glucose. In an aspect, the MHCA is fructose. In an aspect, the MHCA is a mixture of one or more of d-sorbitol, mannitol, xylitol, catechol, fructose, and glucose. In an aspect, the MHCA is a mixture of two or more of d-sorbitol, mannitol, xylitol, catechol, fructose, and glucose. In an aspect, the present disclosure provides for a pharmaceutical composition comprising a particulate metal titanate ion exchanger comprising between 0.01% to 4.0% weight per weight (w/w) of at least one MHCA. In an aspect, the present disclosure provides for a pharmaceutical composition comprising a particulate metal titanate ion exchanger comprising between 0.01% to 2.0% weight per weight (w/w) of at least one MHCA. In an aspect, the present disclosure provides for a pharmaceutical composition comprising a particulate metal titanate ion exchanger comprising between 0.01% to 1.0% weight per weight (w/w) of at least one MHCA. In an aspect, the present disclosure provides for a pharmaceutical composition comprising a particulate metal titanate ion exchanger comprising between 0.01% to 0.6% weight per weight (w/w) of at least one MHCA.

In an aspect, the present disclosure provides for a particulate metal titanate ion exchanger having an empirical formula on an anhydrous basis of: AₘTiₓM_{y}O_{z} wherein A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the particulate metal titanate ion exchanger having been synthesized in the presence of at least one multihydroxyl-containing complexing agent (MHCA), and wherein the particulate metal titanate ion exchanger exhibits a median particle size of greater than 3 microns (μm).

In an aspect, the present disclosure provides for a macroporous particulate titanate ion exchanger having an empirical formula on an anhydrous basis of: AₘTiO_{z}, wherein A is an exchangeable cation that is a potassium ion; “m” is the mole ratio of A to Ti and has a value from 0.10 to 0.60; and “z” is the mole ratio of O to Ti and has a value from 2.05 to 2.60, wherein the macroporous titanate ion exchanger having been synthesized in the presence of a multihydroxyl-containing complexing agent (MHCA) that is d-sorbitol, wherein the macroporous particulate titanate ion exchanger exhibits a median particle size that is between 25 to 125 microns (μm), wherein less than 3.0% of the particles of the macroporous particulate titanate ion exchanger have a particle size less than 3 microns (μm), and wherein the macroporous particulate titanate ion exchanger has a Brunauer-Emmett-Teller (BET) surface area of at least 150 square meters per gram (m²/g).

In an aspect, the present disclosure provides for a macroporous particulate titanate ion exchanger having an empirical formula on an anhydrous basis of: AₘTiO_{z}, wherein A is an exchangeable cation that is a potassium ion; “m” is the mole ratio of A to Ti and has a value from 0.10 to 0.60; and “z” is the mole ratio of O to Ti and has a value from 2.05 to 2.60, wherein the macroporous titanate ion exchanger having been synthesized in the presence of a multihydroxyl-containing complexing agent (MHCA) that is d-sorbitol, wherein the macroporous particulate titanate ion exchanger exhibits a median particle size that is between 25 to 125 microns (μm), wherein less than 0.5% of the particles of the macroporous particulate titanate ion exchanger have a particle size less than 3 microns (μm), and wherein the macroporous particulate titanate ion exchanger has a Brunauer-Emmett-Teller (BET) surface area of at least 150 square meters per gram (m²/g).

### B. Methods

In an aspect, the present disclosure provides for, and includes, a method for manufacturing a tablet or capsule for oral administration, the tablet or capsule comprising a particulate metal titanate ion exchanger having an empirical formula on an anhydrous basis of: AₘTiₓM_{y}O_{z} wherein A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the particulate metal titanate ion exchanger exhibits a median particle size of greater than 3 microns (μm), the method comprising the steps of: (a) forming a reaction mixture comprising reactive sources of A, Ti, at least one multihydroxyl-containing complexing agent (MHCA), optionally M, optionally hydrogen peroxide, optionally a complexing agent (C), and water; (b) heating the reaction mixture for a period of time to form a metal titanate ion exchanger; (c) treating the metal titanate ion exchanger with an alkali base to form the particulate metal titanate ion exchanger; and (d) forming a capsule or tablet comprising the particulate metal titanate ion exchanger, wherein the reaction mixture comprises a composition expressed in terms of mole ratios of the oxides of: p A₂O: a TiO₂:b MO_{q/n}:c H₂O₂:d MHCA:e C:f H₂O wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000. In an aspect, the at least one MHCA is selected from the group consisting of a sugar alcohol, a sugar, and an aromatic compound. In an aspect, the at least one MHCA is d-sorbitol, mannitol, xylitol, catechol, fructose, or glucose. In an aspect, the at least one MHCA is d-sorbitol. In an aspect, the at least one MHCA is mannitol. In an aspect, the at least one MHCA is xylitol. In an aspect, the at least one MHCA is catechol. In an aspect, the at least one MHCA is fructose. In an aspect, the at least one MHCA is glucose. In an aspect, the source of Ti is TiO₂ powder or spray dried TiO₂ spheres. In an aspect, the source of Ti is TiO₂ powder. In an aspect, the source of Ti is preformed spray dried TiO₂ spheres. In an aspect, the source of Ti further comprises Ti(OiPr)₄. In an aspect, C is selected from the group consisting of citric acid, tartaric acid, EDTA, bipyridine, and any combination thereof. In an aspect, C is citric acid. In an aspect, C is tartaric acid. In an aspect, C is EDTA. In an aspect, C is bipyridine. In an aspect, C is a combination of two or more of citric acid, tartaric acid, EDTA, and bipyridine. In an aspect, the hydrogen peroxide is 30 weight percent (wt. %) aqueous hydrogen peroxide. In an aspect, the alkali base is potassium hydroxide. In an aspect, the heating is at about 85° C. to about 225° C. In an aspect, the period of time is between 0.5 days and 30 days. In an aspect, the method for manufacturing a tablet or capsule for oral administration further comprises a step of treating the particulate metal titanate ion exchanger with an acid following step (c). In an aspect, the acid is selected from the group consisting of nitric acid, hydrochloric acid, perchloric acid, and sulfuric acid. In an aspect, the acid is nitric acid. In an aspect, the acid is hydrochloric acid. In an aspect, the acid is perchloric acid. In an aspect, the acid is sulfuric acid. In an aspect, the step of treating the particulate metal titanate ion exchanger with an acid following step (c) is at a pH of between 1 and 3 for at least 15 minutes. In an aspect, the method for manufacturing a tablet or capsule for oral administration further comprises a step of sterilizing the particulate metal titanate ion exchanger. In an aspect, the sterilizing is by means of an autoclave. In an aspect, step (d) of forming a capsule or tablet further comprises adding a binding agent. In an aspect, the binding agent is zirconia. In an aspect, step (d) of forming a capsule or tablet further comprises annealing the particulate metal titanate ion exchanger. In an aspect, the method for manufacturing a tablet or capsule for oral administration further comprises a step of spray drying the particulate metal titanate ion exchanger to form enlarged aggregates of the particulate metal titanate ion exchanger of the prior to forming the capsule or tablet.

In an aspect, the present disclosure provides for, and includes, a method for selectively removing Pb²⁺ toxins from gastrointestinal fluid, the process comprising contacting the fluid containing the toxins with a particulate metal titanate ion exchanger, resulting in an ion exchanged ion exchanger and thereby removing the Pb²⁺ toxins from the fluid, the particulate metal titanate ion exchanger having an empirical formula on an anhydrous basis of: AₘTiₓM_{y}O_{z} wherein A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the metal titanate ion exchanger having been synthesized in the presence of at least one multihydroxyl-containing complexing agent (MHCA), wherein the particulate metal titanate ion exchanger exhibits a median particle size of greater than 3 microns (μm), and wherein the particulate metal titanate ion exchanger reduces the levels of any one or more ions selected from Na⁺, Mg²⁺, K⁺, and Ca²⁺ from the fluid by 3% or less. In an aspect, the particulate metal titanate ion exchanger reduces the levels of any one or more ions selected from Na⁺, Mg²⁺, K⁺, and Ca²⁺ by 2% or less. In an aspect, the particulate metal titanate ion exchanger reduces the levels of any one or more ions selected from Na⁺, Mg²⁺, K⁺, and Ca²⁺ by 1% or less. In an aspect, the particulate metal titanate ion exchanger reduces the levels of any one or more ions selected from Na⁺, Mg²⁺, K⁺, and Ca²⁺ by 0.5% or less. In an aspect, the particulate metal titanate ion exchanger reduces the levels of any one or more ions selected from Na⁺, Mg²⁺, K⁺, and Ca²⁺ between 1% to 3%. In an aspect, the particulate metal titanate ion exchanger reduces the levels of any one or more ions selected from Na⁺, Mg²⁺, K⁺, and Ca²⁺ between 0.5% to 3%. In an aspect, the particulate metal titanate ion exchanger reduces the levels of any one or more ions selected from Na⁺, Mg²⁺, K⁺, and Ca²⁺ between 1% to 2%. In an aspect, the particulate metal titanate ion exchanger reduces the levels of any one or more ions selected from Na⁺, Mg²⁺, K⁺, and Ca²⁺ between 0.5% to 1%. In an aspect, the one or more ions are Na⁺, Mg²⁺, K⁺, and Ca²⁺ In an aspect, the particulate metal titanate ion exchanger does not substantially reduce the levels of any one or more ions selected from Na⁺, Mg²⁺, K⁺, and Ca²⁺ In an aspect, the particulate metal titanate ion exchanger does not substantially reduce the level of Na. In an aspect, the particulate metal titanate ion exchanger does not substantially reduce the level of Mg². In an aspect, the particulate metal titanate ion exchanger does not substantially reduce the level of K. In an aspect, the particulate metal titanate ion exchanger does not substantially reduce the level of Ca²⁺ In an aspect, the particulate metal titanate ion exchanger does not substantially reduce the levels of each of Na⁺, Mg²⁺, K⁺, and Ca²⁺ In an aspect, the levels of the any one or more ions selected from Na⁺, Mg²⁺, K⁺, and Ca²⁺ are measured by inductively coupled plasma (ICP) elemental analysis of the fluid. In an aspect, unbound Pb²⁺ toxins are not detectable in the fluid after the contacting as measured by inductively coupled plasma (ICP) elemental analysis of the fluid. In an aspect, Pb²⁺ toxins are sequestered within the ion exchanged ion exchanger after the contacting. In an aspect, the selective removal of the method for selectively removing Pb²⁺ toxins from gastrointestinal fluid is an intracorporeal process.

In an aspect, the present disclosure provides for, and includes an intracorporcal process for removing Pb²⁺ toxins from gastrointestinal fluid, the process comprising contacting the fluid containing the toxins with a particulate metal titanate ion exchanger resulting in an ion exchanged ion exchanger thereby removing the toxins from the fluid, the particulate metal titanate ion exchanger having an empirical formula on an anhydrous basis of: AₘTiₓM_{y}O_{z}, wherein A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the particulate metal titanate ion exchanger having been synthesized in the presence of at least one multihydroxyl-containing complexing agent (MHCA). In an aspect, the particulate metal titanate ion exchanger product has been annealed at a temperature of 350° C. for 2-6 hours. In an aspect, the particulate metal titanate ion exchanger has been formed into a shaped article for the purpose of oral ingestion. In an aspect, the shaped article is selected from the group consisting of pills, extrudates, spheres, pellets and irregularly shaped particles. In an aspect, the shaped article is a pill. In an aspect, the shaped article is an extrudate. In an aspect, the shaped article is a sphere. In an aspect, the shaped article is a pellet. In an aspect, the shaped article is an irregularly shaped particle.

In an aspect, the present disclosure provides for, and includes a process for preparing a particulate metal titanate ion exchanger, the particulate metal titanate ion exchanger having an empirical formula on an anhydrous basis of: AₘTiₓM_{y}O_{z}, wherein A is an exchangeable cation selected from the group consisting of potassium ion, sodium ion, lithium ion, calcium ion, magnesium ion, hydronium ion or mixtures thereof; M is at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, the process comprising the steps of (a) forming a reaction mixture comprising reactive sources of A, Ti, at least one multihydroxyl-containing complexing agent (MHCA), optionally M, optionally hydrogen peroxide, optionally a complexing agent (C), and water, and (b) heating the reaction mixture at a temperature of about 85° C. to about 225° C. for a time period of 0.5 to 30 days to form the particulate metal titanate ion exchanger, wherein the reaction mixture comprises a composition expressed in terms of mole ratios of the oxides of:

p A₂O: a TiO₂:b MO_{q/2}:c H₂O₂:d MHCA:e C:f H₂O
wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000. In an aspect, the at least one MHCA is d-sorbitol, mannitol, xylitol, catechol, fructose, or glucose. In an aspect, the at least one MHCA is d-sorbitol. In an aspect, the at least one MHCA is mannitol. In an aspect, the at least one MHCA is xylitol. In an aspect, the at least one MHCA is catechol. In an aspect, the at least one MHCA is fructose. In an aspect, the at least one MHCA is glucose. In an aspect, C is selected from the group consisting of citric acid, tartaric acid, EDTA, bipyridine, and any combination thereof. In an aspect, C is citric acid. In an aspect, C is tartaric acid. In an aspect, Cis EDTA. In an aspect, C is bipyridine. In an aspect, C is a combination of two or more of citric acid, tartaric acid, EDTA, and bipyridine. In an aspect, the reaction mixture of step (b) comprises hydrogen peroxide, complexing agent (C), one MHCA, a Ti source that is Ti(OiPr)₄, and optionally M. In an aspect, the reaction mixture of step (b) is a homogenous solution. In an aspect, the Ti source is TiO₂ powder. In an aspect, the Ti source is spray dried TiO₂ spheres. In an aspect, the Ti source is TiO₂ powder, the at least one MHCA is d-sorbitol, M is selected from the group consisting of Fe, Mn, Co, Sn, Zr, and mixtures thereof, and C s citric acid. In an aspect, the Ti source is TiO₂ powder, optionally including an additional Ti source that is Ti(OiPr)₄, the at least one MHCA is d-sorbitol, the hydrogen peroxide is 30 weight percent (wt. %) hydrogen peroxide, M is selected from the group consisting of Fe, Mn, Co, Sn, Zr, Nb, and mixtures thereof, and C is citric acid. In an aspect, the Ti source is spray dried TiO₂ spheres, the at least one MHCA is d-sorbitol, M is Fe, Mn, Co, Sn, Zr, or mixtures thereof, and C is citric acid. In an aspect, the Ti source is spray dried TiO₂ spheres, optionally including an additional Ti source that is Ti(OiPr)₄, the at least one MHCA is d-sorbitol, the hydrogen peroxide is 30 weight percent (wt. %) hydrogen peroxide, M is Fe, Mn, Co, Sn, Zr, Nb, or mixtures thereof, and C is citric acid.

### C. Definitions

The term “and/or” when used in a list of two or more items, means that any one of the listed items can be employed by itself or in combination with any one or more of the listed items. For example, the expression “A and/or B” is intended to mean either or both of A and B, i.e., A alone, B alone, or A and B in combination. The expression “A, B and/or C” is intended to mean A alone, B alone, C alone, A and B in combination, A and C in combination, B and C in combination, or A, B, and C in combination.

As used herein, terms in the singular and the singular forms “a,” “an,” and “the,” for example, include plural referents unless the content clearly dictates otherwise.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. Whenever the phrase “comprising” is used, variations such as “consisting essentially of” and “consisting of” are also contemplated.

Unless defined otherwise herein, terms are to be understood according to conventional usage by those of ordinary skill in the relevant art. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), or the “McGraw-Hill Dictionary of Scientific and Technical Terms” (6th edition, 2002, McGraw-Hill, New York).

As used herein, the term “about” refers to a range extending to +/−10% of the specified value.

As used herein, “metallate” refers to a complex anionic compound comprising one or more metal atoms ligated to one or more non-metal atoms. A metallate may further comprise one or more cations. In an aspect, one or more metal atoms of a metallate are selected from titanium (Ti), niobium (Nb), zirconium (Zr), tin (Sn), cobalt (Co), and manganese (Mn). In an aspect, one or more non-metal atoms are selected from oxygen (O) and sulfur(S). In an aspect, one or more cations of a metallate are selected from potassium ion, sodium ion, lithium ion, calcium ion, magnesium ion, and hydronium ion. In an aspect, a metallate is inorganic. In an aspect, a metallate comprises titanium, oxygen, and potassium.

As used herein, “nano-sized titania” or “nano-sized TiO₂” refers to titanium dioxide reagents with crystallite size or particle size less than 200 nm across, preferably about 100 nm across or less. Any form of titania can be nano-sized titania, including nano-sized anatase, nano-sized rutile, nano-sized brookite, nano-sized amorphous titania, and nano-sized titanium oxyhydroxide. Nano-sized titania sources may be used to make preformed spay dried sphere reagents.

As used herein, “ion exchanger” refers to a complex wherein one or more charged species may exchange with one or more charged species of the surrounding environment. In an aspect, an ion exchanger is a cation exchanger.

As used herein, “morphology” refers to form or shape of a particulate. A particulate's morphology may include, but is not limited to, spheres, interpenetrating spheres, fibers, slabs, intertwined plates, and amorphous morphology.

As used herein, “spherical morphology” refers to particulate morphology that is substantially and discernibly spherical in form.

As used herein, “amorphous morphology” refers to particulate morphology that is substantially and discernibly absent of order or repeating form.

As used herein, “macroporous” refers to the porosity of a particulate wherein pore diameter is greater than about 50 nanometers (nm).

As used herein, “polycrystalline” refers to the crystallinity of a material comprising multiple crystallite of varying orientation and size.

As used herein, “polycrystalline aggregate” refers to the crystallinity of an aggregate material comprising several crystallite of varying orientation and size.

As used herein a “Brunauer-Emmett-Teller (BET)” surface area refers to the specific surface area of a solid porous material characterized by Brunauer-Emmett-Teller (BET) analysis which is based on gas adsorption measurements. See Brunauer et al., “Adsorption of Gas in Multimolecular Layers,” J. Am. Chem. Soc. 60 (2): 309-319. Without being by bound theory, in a BET analysis, the true or specific surface area of a porous solid particle, including surface irregularities and pore walls, is determined at an atomic level by adsorption of an unreactive gas. The BET equation calculates surface coverage θ, where (p/pₒ) is the relative pressure, and c is a BET C-constant related to the heat of adsorption:

$$θ = \frac{c p}{( 1 - p / {p}_{o} ) ( {p}_{o} + p ( c - 1 ) )} .$$

As used herein “intracorporeal” refers to a process occurring within the body of a subject or patient. For example, an intracorporeal process for removing Pb²⁺ toxins refers to the process for removing Pb²⁺ from fluids, such as gastrointestinal fluids, within the body of a subject or patient.

As used herein “annealing” refers to a process of heat treating a material at one or more elevated temperatures for one or more pre-determined periods of time, where the annealing may alter one or more physical or chemical properties of the material, including, but not limited to, crystallinity, particle morphology, particle size distribution, and porosity. Annealing parameters/conditions may selectively alter one or more physical or chemical properties of a material and may include, but are not limited to, ramp rate, peak temperature, holding time, cooling rate, and gas environment. A gas environment for an annealing process may be selected from, but is not limited to, atmosphere, and an inert gas, such as nitrogen (N₂) or argon (Ar).

As used herein a “spray-drying” refers to a technique of forming a substantially homogenous dry powder from a liquid mixture or slurry, by drying a sprayed liquid mixture or slurry in the presence of a heated gas. The heated gas may be heated atmospheric gas or a heated inert gas, such as nitrogen (N₂) or argon (Ar).

All publications, patents, and patent applications mentioned in this disclosure are herein incorporated by reference to the same extent as if each individual publication, patent, or patent application was specifically and individually indicated to be incorporated by reference.

Having now generally described the disclosure, the same will be more readily understood through reference to the following embodiments and examples that are provided by way of illustration and are not intended to be limiting of the present disclosure, unless specified.

### Embodiments

Embodiment 1. An intracorporeal process for removing Pb²⁺ toxins from gastrointestinal fluid, the process comprising contacting the fluid containing the toxins with a particulate metal titanate ion exchanger resulting in an ion exchanged ion exchanger thereby removing the toxins from the fluid, the particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the particulate metal titanate ion exchanger having been synthesized in the presence of at least one multihydroxyl-containing complexing agent (MHCA).

Embodiment 2. The process of embodiment 1, wherein the multihydroxyl-containing complexing agent (MHCA) is selected from the group consisting of d-sorbitol, mannitol, xylitol, catechol, fructose, glucose, and mixtures thereof.

Embodiment 3. The process of embodiment 1 or embodiment 2, wherein the particulate metal titanate ion exchanger exhibits a median particle size greater than 3 microns (μm).

Embodiment 4. The process of any one of embodiments 1 to 3, wherein the particulate metal titanate ion exchanger exhibits a median particle size ranging from 25 to 125 microns (μm).

Embodiment 5. The process of any one of embodiments 1 to 4, wherein less than 3% of the particles of the particulate metal titanate ion exchanger exhibit a particle size less than 3 microns (μm).

Embodiment 6. The process of embodiment 5, wherein less than 0.5% of the particles of the particulate metal titanate ion exchanger exhibit a particle size less than 3 microns (μm).

Embodiment 7. The process of any one of embodiments 1 to 6, wherein the particulate metal titanate ion exchanger has a Brunauer-Emmett-Teller (BET) surface area greater than 150 square meters per gram (m²/g).

Embodiment 8. The process of any one of embodiments 1 to 7, wherein the particulate metal titanate ion exchanger product having been annealed at a temperature of 350° C. for 2-6 hours.

Embodiment 9. The process of any one of embodiments 1 to 8, wherein the particulate metal titanate ion exchanger having been formed into a shaped article to be ingested orally.

Embodiment 10. The process of embodiment 9, wherein said shaped article is selected from the group consisting of pills, extrudates, spheres, pellets and irregularly shaped particles.

Embodiment 11. A process for preparing a particulate metal titanate ion exchanger, the particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, the process comprising the steps of
   (a) forming a reaction mixture comprising reactive sources of A, Ti, at least one multihydroxyl-containing complexing agent (MHCA), optionally M, optionally hydrogen peroxide, optionally a complexing agent (C), and water, and
   (b) heating the reaction mixture at a temperature of about 85° C. to about 225° C. for a time period of 0.5 to 30 days to form the particulate metal titanate ion exchanger, wherein the reaction mixture comprises a composition expressed in terms of mole ratios of the oxides of:

p A₂O: a TiO₂:b MO_{q/n}:c H₂O₂:d MHCA:e C:f H₂O
wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000.

Embodiment 12. The process of embodiment 11, wherein the at least one multihydroxyl-containing complexing agent (MHCA) is selected from the group consisting of d-sorbitol, mannitol, xylitol, catechol, fructose, glucose, and mixtures thereof.

Embodiment 13. The process of embodiment 11 or embodiment 12, wherein C is citric acid, tartaric acid, EDTA, bipyridine, or mixtures thereof.

Embodiment 14. The process of any one of embodiments 11 to 13, wherein the reaction mixture comprises hydrogen peroxide, C, one multihydroxyl-containing complexing agent (MHCA), a Ti source being Ti(OiPr)₄, and optionally M.

Embodiment 15. The process of any one of embodiments 11 to 14, wherein the reaction mixture is a homogenous solution.

Embodiment 16. The process of any one of embodiments 11 to 13, wherein the Ti source is TiO₂ powder, including nano-sized titania.

Embodiment 17. The process of any one of embodiments 11 to 13, wherein the Ti source is TiO₂ powder, the at least one multihydroxyl-containing complexing agent (MHCA) is d-sorbitol, M is selected from the group consisting of Fe, Mn, Co, Sn, Zr, and mixtures thereof, and C s citric acid.

Embodiment 18. The process of any one of embodiments 11 to 13, wherein the Ti source is TiO₂ powder, optionally including an additional Ti source that is Ti(OiPr)₄, the at least one multihydroxyl-containing complexing agent (MHCA) is d-sorbitol, the hydrogen peroxide is 30 weight percent (wt. %) hydrogen peroxide, M is selected from the group consisting of Fe, Mn, Co, Sn, Zr, Nb, and mixtures thereof, and C is citric acid.

Embodiment 19. The process of any one of embodiments 11 to 13, wherein the Ti source is preformed spray dried TiO₂ spheres.

Embodiment 20. The process of any one of embodiments 11 to 13, wherein the Ti source is preformed spray dried TiO₂ spheres, the at least one multihydroxyl-containing complexing agent (MHCA) is d-sorbitol, M is Fe, Mn, Co, Sn, Zr, or mixtures thereof, and C is citric acid.

Embodiment 21. The process of any one of embodiments 11 to 13, wherein the Ti source is spray dried TiO₂ spheres, optionally including an additional Ti source that is Ti(OiPr)₄, the at least one multihydroxyl-containing complexing agent (MHCA) is d-sorbitol, the hydrogen peroxide is 30 weight percent (wt. %) hydrogen peroxide, M is Fe, Mn, Co, Sn, Zr, Nb, or mixtures thereof, and C is citric acid.

Embodiment 22. A particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85,
wherein the particulate metal titanate ion exchanger having been synthesized in the presence of at least one multi-hydroxy complexing agent (MHCA), and wherein the particulate metal titanate ion exchanger exhibits a median particle size of greater than 3 microns (μm).

Embodiment 23. The ion exchanger of embodiment 22, wherein the particulate metal titanate ion exchanger is an acid-treated particulate metal titanate ion exchanger.

Embodiment 24. The ion exchanger of embodiment 22 or embodiment 23, wherein A is potassium ion, hydronium ion, or a mixture thereof.

Embodiment 25. The ion exchanger of any one of embodiments 22 to 24, wherein A is potassium ion.

Embodiment 26. The ion exchanger of embodiment 22 or embodiment 23, wherein A is sodium ion.

Embodiment 27. The ion exchanger of any one of embodiments 22 to 24, wherein A is a mixture of potassium and hydronium ions.

Embodiment 28. The ion exchanger of any one of embodiments 22 to 27, wherein the particulate metal titanate ion exchanger is a polycrystalline aggregate metal titanate ion exchanger.

Embodiment 29. The ion exchanger of any one of embodiments 22 to 28, wherein the particulate metal titanate ion exchanger is macroporous.

Embodiment 30. The ion exchanger of any one of embodiments 22 to 29, wherein the particulate metal titanate ion exchanger has spherical morphology.

Embodiment 31. The ion exchanger of any one of embodiments 22 to 29, wherein the particulate metal titanate ion exchanger has amorphous morphology.

Embodiment 32. The ion exchanger of any one of embodiments 22 to 31, wherein the particulate metal titanate ion exchanger is a powder.

Embodiment 33. The ion exchanger of any one of embodiments 22 to 32, wherein the median particle size is between 25 to 125 microns (μm).

Embodiment 34. The ion exchanger of any one of embodiments 22 to 33, wherein less than 3% of the particles of the particulate metal titanate ion exchanger have a particle size of less than 3 microns (μm).

Embodiment 35. The ion exchanger of embodiment 34, wherein less than 0.5% of the particles of the particulate metal titanate ion exchanger have a particle size less than 3 microns (μm).

Embodiment 36. The ion exchanger of any one of embodiments 22 to 35, wherein the particulate metal titanate ion exchanger has a particle size distribution d₁₀ value of between about 5 microns (μm) and about 70 μm.

Embodiment 37. The ion exchanger of embodiment 36, wherein the particulate metal titanate ion exchanger has a particle size distribution d₁₀ value of between about 12 μm and about 18 μm.

Embodiment 38. The ion exchanger of embodiment 37, wherein the particulate metal titanate ion exchanger has a particle size distribution d₁₀ value of about 15 μm.

Embodiment 39. The ion exchanger of embodiment 36, wherein the particulate metal titanate ion exchanger has a particle size distribution d₁₀ value of between about 42 μm and about 48 μm.

Embodiment 40. The ion exchanger of embodiment 39, wherein the particulate metal titanate ion exchanger has a particle size distribution d₁₀ value of about 45 μm.

Embodiment 41. The ion exchanger of embodiment 36, wherein the particulate metal titanate ion exchanger has a particle size distribution d₁₀ value of between about 51 μm and about 57 μm.

Embodiment 42. The ion exchanger of embodiment 41, wherein the particulate metal titanate ion exchanger has a particle size distribution d₁₀ value of about 54 μm.

Embodiment 43. The ion exchanger of any one of embodiments 22 to 42, wherein the particulate metal titanate ion exchanger has a particle size distribution d₅₀ value of between about 25 microns (μm) and about 125 μm.

Embodiment 44. The ion exchanger of embodiment 43, wherein the particulate metal titanate ion exchanger has a particle size distribution d₅₀ value of between about 48 μm and about 54 μm.

Embodiment 45. The ion exchanger of embodiment 44, wherein the particulate metal titanate ion exchanger has a particle size distribution d₅₀ value of about 51 μm.

Embodiment 46. The ion exchanger of embodiment 43, wherein the particulate metal titanate ion exchanger has a particle size distribution d₅₀ value of between about 71 μm and about 77 μm.

Embodiment 47. The ion exchanger of embodiment 46, wherein the particulate metal titanate ion exchanger has a particle size distribution d₅₀ value of about 74 μm.

Embodiment 48. The ion exchanger of embodiment 43, wherein the particulate metal titanate ion exchanger has a particle size distribution d₅₀ value of between about 89 μm and about 95 μm.

Embodiment 49. The ion exchanger of embodiment 48, wherein the particulate metal titanate ion exchanger has a particle size distribution d₅₀ value of about 92 μm.

Embodiment 50. The ion exchanger of any one of embodiments 22 to 49, wherein the particulate metal titanate ion exchanger has a particle size distribution d₉₀ value of between about 55 microns (μm) and about 185 μm.

Embodiment 51. The ion exchanger of embodiment 50, wherein the particulate metal titanate ion exchanger has a particle size distribution d₉₀ value of between about 99 μm and about 105 μm.

Embodiment 52. The ion exchanger of embodiment 51, wherein the particulate metal titanate ion exchanger has a particle size distribution d₉₀ value of about 102 μm.

Embodiment 53. The ion exchanger of embodiment 50, wherein the particulate metal titanate ion exchanger has a particle size distribution d₉₀ value of between about 137 μm and about 143 μm.

Embodiment 54. The ion exchanger of embodiment 53, wherein the particulate metal titanate ion exchanger has a particle size distribution d₉₀ value of about 140 μm.

Embodiment 55. The ion exchanger of embodiment 50, wherein the particulate metal titanate ion exchanger has a particle size distribution d₉₀ value of between about 156 μm and about 162 μm.

Embodiment 56. The ion exchanger of embodiment 55, wherein the particulate metal titanate ion exchanger has a particle size distribution d₉₀ value of about 159 μm.

Embodiment 57. The ion exchanger of any one of embodiments 22 to 56, wherein the particulate metal titanate ion exchanger is stable in a liquid environment at a pH of 1-2.

Embodiment 58. The ion exchanger of any one of embodiments 22 to 57, wherein the particulate metal titanate ion exchanger has a Brunauer-Emmett-Teller (BET) surface area of greater than 150 square meters per gram (m²/g).

Embodiment 59. The ion exchanger of embodiment 58, wherein the particulate metal titanate ion exchanger has a BET surface area of about 197 m²/g.

Embodiment 60. The ion exchanger of any one of embodiments 22 to 57, wherein the particulate metal titanate ion exchanger has a Brunauer-Emmett-Teller (BET) surface area of greater than 200 square meters per gram (m²/g).

Embodiment 61. The ion exchanger of embodiment 60, wherein the particulate metal titanate ion exchanger has a BET surface area of about 203 m²/g.

Embodiment 62. The ion exchanger of any one of embodiments 22 to 57, wherein the particulate metal titanate ion exchanger has a Brunauer-Emmett-Teller (BET) surface area of greater than 230 square meters per gram (m²/g).

Embodiment 63. The ion exchanger of embodiment 62, wherein the particulate metal titanate ion exchanger has a BET surface area of about 236 m²/g.

Embodiment 64. The ion exchanger of any one of embodiments 22 to 63, wherein the particulate metal titanate ion exchanger is substantially insoluble at a pH range of 1-7.

Embodiment 65. The ion exchanger of any one of embodiments 22 to 63, wherein the particulate metal titanate ion exchanger is substantially insoluble at a pH range of 7-13.

Embodiment 66. The ion exchanger of any one of embodiments 22 to 63, wherein the particulate metal titanate ion exchanger is substantially insoluble at a pH range of 1-13.

Embodiment 67. The ion exchanger of any one of embodiments 22 to 63, wherein the particulate metal titanate ion exchanger is substantially insoluble at physiological pH.

Embodiment 68. The ion exchanger of any one of embodiments 22 to 63, and 67, wherein the particulate metal titanate ion exchanger is substantially insoluble at stomach pH.

Embodiment 69. The ion exchanger of any one of embodiments 22 to 68, wherein the particulate metal titanate ion exchanger is substantially insoluble in one or more bodily fluids.

Embodiment 70. The ion exchanger of embodiment 69, wherein the one or more bodily fluids are selected from the group consisting of blood, urine, and gastrointestinal fluid.

Embodiment 71. The ion exchanger of any one of embodiments 22 to 70, wherein the particulate metal titanate ion exchanger has a distribution coefficient (K_{d}) for Pb²⁺ of between about 50,000 to about 5,500,000 milliliters per gram (mL/g) in solution.

Embodiment 72. The ion exchanger of embodiment 71, wherein the particulate metal titanate ion exchanger has a K_{d} for Pb²⁺ of between about 100,000 to about 2,500,000 milliliters per gram (mL/g).

Embodiment 73. The ion exchanger of embodiment 72, wherein the particulate metal titanate ion exchanger has a K_{d} for Pb²⁺ of about 967,800.

Embodiment 74. The ion exchanger of embodiment 72, wherein the particulate metal titanate ion exchanger has a K_{d} for Pb²⁺ of about 809,500.

Embodiment 75. The ion exchanger of embodiment 72, wherein the particulate metal titanate ion exchanger has a K_{d} for Pb²⁺ of about 495,800.

Embodiment 76. The ion exchanger of embodiment 72, wherein the particulate metal titanate ion exchanger has a K_{d} for Pb²⁺ of about 321,900.

Embodiment 77. The ion exchanger of any one of embodiments 22 to 76, wherein the at least one MHCA is selected from the group consisting of a sugar alcohol, a sugar, an aromatic compound, and any combination thereof.

Embodiment 78. The ion exchanger of embodiment 77, wherein the at least one MHCA is a sugar alcohol.

Embodiment 79. The ion exchanger of embodiment 78, wherein the sugar alcohol is selected from the group consisting of d-sorbitol, mannitol, and xylitol.

Embodiment 80. The ion exchanger of any one of embodiments 77 to 79, wherein the at least one MHCA is d-sorbitol.

Embodiment 81. The ion exchanger of embodiment 77, wherein the at least one MHCA is a sugar.

Embodiment 82. The ion exchanger of embodiment 81, wherein the sugar is glucose or fructose.

Embodiment 83. The ion exchanger of embodiment 77, wherein the at least one MHCA is an aromatic compound.

Embodiment 84. The ion exchanger of embodiment 83, wherein the aromatic compound is catechol.

Embodiment 85. The ion exchanger of any one of embodiments 22 to 84, wherein the particulate metal titanate ion exchanger comprises between 0.01% to 4.0% weight per weight (w/w) of the at least one MHCA.

Embodiment 86. The ion exchanger of embodiment 85, wherein the particulate metal titanate ion exchanger comprises between 0.01% to 0.6% w/w of the at least one MHCA.

Embodiment 87. The ion exchanger of any one of embodiments 22 to 86, wherein x is 1 and y is 0.

Embodiment 88. The ion exchanger of any one of embodiments 22 to 87, wherein m is between 0.10 to 0.50.

Embodiment 89. The ion exchanger of embodiment 88, wherein m is about 0.40.

Embodiment 90. The ion exchanger of embodiment 88, wherein m is about 0.30.

Embodiment 91. The ion exchanger of embodiment 88, wherein m is about 0.28.

Embodiment 92. The ion exchanger of any one of embodiments 22 to 91, wherein the x-ray diffraction (XRD) pattern of the particulate metal titanate ion exchanger has a characteristic diffraction line with a d-spacing of 3.00 to 3.11 Å.

Embodiment 93. The ion exchanger of any one of embodiments 22 to 92, wherein the particulate metal titanate ion exchanger has an x-ray diffraction (XRD) pattern having characteristic diffraction lines within the ranges provided below in Table A or Table B:

TABLE A

| 2Θ | d(Å) | I/I₀ |
|---|---|---|
| 11.11-10.78 | 7.96-8.2 | w-m |
| 25.43-24.03 | 3.50-3.70 | w-m |
| 29.55-28.68 | 3.02-3.11 | vs |
| 33.93-33.15 | 2.64-2.70 | m-s |
| 43.36-42.51 | 2.085-2.125 | w-m |
| 48.38-47.31 | 1.88-1.92 | m-s |
| 60.46-59.18 | 1.53-1.56 | w-m |
| 67.03-65.44 | 1.395-1.425 | w-m |

TABLE B

| 2Θ | d(Å) | I/I₀ |
|---|---|---|
| 11.47-10.95 | 7.71-8.07 | w-s |
| 24.37-23.97 | 3.65-3.71 | m-s |
| 29.76-28.78 | 3.00-3.10 | vs |
| 33.80-33.15 | 2.65-2.70 | w-m |
| 43.47-42.51 | 2.08-2.125 | m-s |
| 48.51-47.49 | 1.875-1.913 | m-vs |
| 66.93-65.55 | 1.397-1.423 | w-m. |

Embodiment 94. The ion exchanger of any one of embodiments 22 to 91, wherein the x-ray diffraction (XRD) pattern of the particulate metal titanate ion exchanger has a characteristic diffraction line with a d-spacing of 3.00 to 3.10 Å.

Embodiment 95. The ion exchanger of any one of embodiments 22 to 91, and 94, wherein the particulate metal titanate ion exchanger has an x-ray diffraction (XRD) pattern having characteristic diffraction lines within the ranges provided below in Table C or Table D:

TABLE C

| 2Θ | d(Å) | I/I₀ |
|---|---|---|
| 11.87-10.89 | 7.45-8.12 | w-s |
| 24.50-23.71 | 3.63-3.75 | w-m |
| 29.76-28.87 | 3.00-3.09 | vs |
| 32.06-30.81 | 2.79-2.90 | w-s |
| 33.93-33.15 | 2.64-2.70 | m-s |
| 43.26-42.61 | 2.09-2.12 | w-s |
| 48.24-47.44 | 1.885-1.915 | w-s |
| 60.03-58.77 | 1.54-1.57 | w-m |
| 66.77-65.71 | 1.40-1.42 | w-m |

TABLE D

| 2Θ | d(Å) | I/I₀ |
|---|---|---|
| 11.76-10.82 | 7.52-8.17 | w-s |
| 24.64-23.58 | 3.61-3.77 | w-m |
| 29.76-28.78 | 3.00-3.10 | vs |
| 32.05-30.38 | 2.79-2.94 | w-s |
| 34.06-33.15 | 2.63-2.70 | w-m |
| 43.36-42.51 | 2.085-2.125 | m-vs |
| 48.51-47.44 | 1.875-1.915 | m-vs |
| 60.24-58.76 | 1.535-1.57 | w-m |
| 66.76-65.70 | 1.40-1.42 | w-m. |

Embodiment 96. A macroporous particulate titanate ion exchanger having an empirical formula on an anhydrous basis of:

AₘTiO_{z}
wherein
A is an exchangeable cation selected from the group consisting of potassium ion, hydronium ion, and a mixture thereof; “m” is the mole ratio of A to Ti and has a value from 0.10 to 0.60; and “z” is the mole ratio of O to Ti and has a value from 2.05 to 2.60,
wherein the macroporous titanate ion exchanger having been synthesized in the presence of a multi-hydroxy complexing agent (MHCA) that is d-sorbitol, wherein the macroporous particulate titanate ion exchanger exhibits a median particle size that is between 25 to 125 microns (μm), wherein less than 3.0% of the particles of the macroporous particulate titanate ion exchanger have a particle size less than 3 microns (μm), and wherein the macroporous particulate titanate ion exchanger has a Brunauer-Emmett-Teller (BET) surface area of at least 150 square meters per gram (m²/g).

Embodiment 97. The ion exchanger of embodiment 96, wherein the particulate metal titanate ion exchanger is an acid-treated particulate metal titanate ion exchanger.

Embodiment 98. The ion exchanger of embodiment 96 or embodiment 97, wherein A is potassium ion.

Embodiment 99. The ion exchanger of embodiment 96 or embodiment 97, wherein A is hydronium ion.

Embodiment 100. The ion exchanger of embodiment 96 or embodiment 97, wherein A is a mixture of potassium and hydronium ions.

Embodiment 101. The ion exchanger of any one of embodiments 96 to 100, wherein the macroporous particulate titanate ion exchanger is a polycrystalline aggregate titanate ion exchanger.

Embodiment 102. The ion exchanger of any one of embodiments 96 to 101, wherein the macroporous particulate titanate ion exchanger has amorphous morphology.

Embodiment 103. The ion exchanger of any one of embodiments 96 to 102, wherein the macroporous particulate titanate ion exchanger is a powder.

Embodiment 104. The ion exchanger of any one of embodiments 96 to 103, wherein about 2.1% of the particles of the particulate metal titanate ion exchanger have a particle size of less than 3 microns (μm).

Embodiment 105. The ion exchanger of any one of embodiments 96 to 104, wherein the macroporous particulate titanate ion exchanger has a particle size distribution d₁₀ value of between about 5 microns (μm) and about 45 μm.

Embodiment 106. The ion exchanger of embodiment 105, wherein the macroporous particulate titanate ion exchanger has a particle size distribution d₁₀ value of between about 12 μm and about 18 μm.

Embodiment 107. The ion exchanger of embodiment 106, wherein the macroporous particulate titanate ion exchanger has a particle size distribution d₁₀ value of about 15 μm.

Embodiment 108. The ion exchanger of any one of embodiments 96 to 107, wherein the macroporous particulate titanate ion exchanger has a particle size distribution d₅₀ value of between about 25 microns (μm) and about 75 μm.

Embodiment 109. The ion exchanger of embodiment 108, wherein the macroporous particulate titanate ion exchanger has a particle size distribution d₅₀ value of between about 48 μm and about 54 μm.

Embodiment 110. The ion exchanger of embodiment 109, wherein the macroporous particulate titanate ion exchanger has a particle size distribution d₅₀ value of about 51 μm.

Embodiment 111. The ion exchanger of any one of embodiments 96 to 110, wherein the macroporous particulate titanate ion exchanger has a particle size distribution d₉₀ value of between about 55 microns (μm) and about 140 μm.

Embodiment 112. The ion exchanger of embodiment 111, wherein the macroporous particulate titanate ion exchanger has a particle size distribution d₉₀ value of between about 99 μm and about 105 μm.

Embodiment 113. The ion exchanger of embodiment 112, wherein the macroporous particulate titanate ion exchanger has a particle size distribution d₉₀ value of about 102 μm.

Embodiment 114. The ion exchanger of any one of embodiments 96 to 113, wherein the macroporous particulate titanate ion exchanger is stable in a liquid environment at a pH of 1-2.

Embodiment 115. The ion exchanger of any one of embodiments 96 to 114, wherein the macroporous particulate titanate ion exchanger has a BET surface area of about 197 m²/g.

Embodiment 116. The ion exchanger of any one of embodiments 96 to 114, wherein the macroporous particulate titanate ion exchanger has a BET surface area of about 236 m²/g.

Embodiment 117. The ion exchanger of any one of embodiments 96 to 116, wherein the particulate metal titanate ion exchanger is substantially insoluble at a pH range of 1-7.

Embodiment 118. The ion exchanger of any one of embodiments 96 to 116, wherein the particulate metal titanate ion exchanger is substantially insoluble at a pH range of 7-13.

Embodiment 119. The ion exchanger of any one of embodiments 96 to 116, wherein the particulate metal titanate ion exchanger is substantially insoluble at a pH range of 1-13.

Embodiment 120. The ion exchanger of any one of embodiments 96 to 116, wherein the particulate metal titanate ion exchanger is substantially insoluble at physiological pH.

Embodiment 121. The ion exchanger of any one of embodiments 96 to 116, and 120, wherein the particulate metal titanate ion exchanger is substantially insoluble at stomach pH.

Embodiment 122. The ion exchanger of any one of embodiments 96 to 121, wherein the macroporous particulate titanate ion exchanger is substantially insoluble in one or more bodily fluids.

Embodiment 123. The ion exchanger of embodiment 122, wherein the one or more bodily fluids are selected from the group consisting of blood, urine, and gastrointestinal fluid.

Embodiment 124. The ion exchanger of any one of embodiments 96 to 123, wherein the macroporous particulate titanate ion exchanger has a distribution coefficient (K_{d}) for Pb²⁺ of between about 100,000 to about 2,500,000 milliliters per gram (mL/g).

Embodiment 125. The ion exchanger of embodiment 124, wherein the macroporous particulate titanate ion exchanger has a K_{d} for Pb²⁺ of about 809,500.

Embodiment 126. The ion exchanger of embodiment 124, wherein the macroporous particulate titanate ion exchanger has a K_{d} for Pb²⁺ of about 495,800.

Embodiment 127. The ion exchanger of any one of embodiments 96 to 126, wherein the macroporous particulate titanate ion exchanger comprises between 0.01% to 4.0% weight per weight (w/w) of the MHCA.

Embodiment 128. The ion exchanger of embodiment 127, wherein the particulate metal titanate ion exchanger comprises between 0.01% to 0.6% w/w of the MHCA.

Embodiment 129. The ion exchanger of any one of embodiments 96 to 128, wherein m is about 0.40.

Embodiment 130. The ion exchanger of any one of embodiments 96 to 128, wherein m is about 0.30.

Embodiment 131. A macroporous particulate titanate ion exchanger having an empirical formula on an anhydrous basis of:

AₘTiO_{z}
wherein
A is an exchangeable cation selected from the group consisting of potassium ion, hydronium ion, and a mixture thereof; “m” is the mole ratio of A to Ti and has a value from 0.10 to 0.60; and “z” is the mole ratio of O to Ti and has a value from 2.05 to 2.60,
wherein the macroporous particulate titanate ion exchanger having been synthesized in the presence of a multi-hydroxy complexing agent (MHCA) that is d-sorbitol, wherein the macroporous particulate titanate ion exchanger exhibits a median particle size that is between 25 to 125 microns (μm), wherein less than 0.5% of the particles of the macroporous particulate titanate ion exchanger have a particle size less than 3 microns (μm), and wherein the macroporous particulate titanate ion exchanger has a Brunauer-Emmett-Teller (BET) surface area of at least 150 square meters per gram (m²/g).

Embodiment 132. The ion exchanger of embodiment 131, wherein the particulate metal titanate ion exchanger is an acid-treated particulate metal titanate ion exchanger.

Embodiment 133. The ion exchanger of embodiment 131 or embodiment 132, wherein A is potassium ion.

Embodiment 134. The ion exchanger of embodiment 131 or embodiment 132, wherein A is hydronium ion.

Embodiment 135. The ion exchanger of embodiment 131 or embodiment 132, wherein A is a mixture of potassium and hydronium ions.

Embodiment 136. The ion exchanger of any one of embodiments 131 to 135, wherein the macroporous particulate titanate ion exchanger is a polycrystalline aggregate titanate ion exchanger.

Embodiment 137. The ion exchanger of any one of embodiments 131 to 136, wherein the macroporous particulate titanate ion exchanger has spherical morphology.

Embodiment 138. The ion exchanger of any one of embodiments 131 to 137, wherein the macroporous particulate titanate ion exchanger is a powder.

Embodiment 139. The ion exchanger of any one of embodiments 131 to 138, wherein the macroporous particulate titanate ion exchanger has a particle size distribution d₁₀ value of between about 30 microns (μm) and about 70 μm.

Embodiment 140. The ion exchanger of embodiment 139, wherein the macroporous particulate titanate ion exchanger has a particle size distribution d₁₀ value of between about 42 μm and about 48 μm.

Embodiment 141. The ion exchanger of embodiment 140, wherein the macroporous particulate titanate ion exchanger has a particle size distribution d₁₀ value of about 45 μm.

Embodiment 142. The ion exchanger of embodiment 139, wherein the macroporous particulate titanate ion exchanger has a particle size distribution d₁₀ value of between about 51 μm and about 57 μm.

Embodiment 143. The ion exchanger of embodiment 142, wherein the macroporous particulate titanate ion exchanger has a particle size distribution d₁₀ value of about 54 μm.

Embodiment 144. The ion exchanger of any one of embodiments 131 to 143, wherein the macroporous particulate titanate ion exchanger has a particle size distribution d₅₀ value of between about 55 microns (μm) and about 125 μm.

Embodiment 145. The ion exchanger of embodiment 144, wherein the macroporous particulate titanate ion exchanger has a particle size distribution d₅₀ value of between about 71 μm and about 77 μm.

Embodiment 146. The ion exchanger of embodiment 145, wherein the particulate metal titanate ion exchanger has a particle size distribution d₅₀ value of about 74 μm.

Embodiment 147. The ion exchanger of embodiment 144, wherein the macroporous particulate titanate ion exchanger has a particle size distribution d₅₀ value of between about 89 μm and about 95 μm.

Embodiment 148. The ion exchanger of embodiment 147, wherein the particulate metal titanate ion exchanger has a particle size distribution d₅₀ value of about 92 μm.

Embodiment 149. The ion exchanger of any one of embodiments 131 to 148, wherein the macroporous particulate titanate ion exchanger has a particle size distribution d₉₀ value of between about 120 microns (μm) and about 180 μm.

Embodiment 150. The ion exchanger of embodiment 149, wherein the macroporous particulate titanate ion exchanger has a particle size distribution d₉₀ value of between about 137 μm and about 143 μm.

Embodiment 151. The ion exchanger of embodiment 150, wherein the particulate metal titanate ion exchanger has a particle size distribution d₉₀ value of about 140 μm.

Embodiment 152. The ion exchanger of embodiment 149, wherein the particulate metal titanate ion exchanger has a particle size distribution d₉₀ value of between about 156 μm and about 162 μm.

Embodiment 153. The ion exchanger of embodiment 152, wherein the particulate metal titanate ion exchanger has a particle size distribution d₉₀ value of about 159 μm.

Embodiment 154. The ion exchanger of any one of embodiments 131 to 153, wherein the macroporous particulate titanate ion exchanger is stable in a liquid environment at a pH of 1-2.

Embodiment 155. The ion exchanger of any one of embodiments 131 to 154, wherein the macroporous particulate titanate ion exchanger has a BET surface area of about 203 m²/g.

Embodiment 156. The ion exchanger of any one of embodiments 131 to 155, wherein the particulate metal titanate ion exchanger is substantially insoluble at a pH range of 1-7.

Embodiment 157. The ion exchanger of any one of embodiments 131 to 155, wherein the particulate metal titanate ion exchanger is substantially insoluble at a pH range of 7-13.

Embodiment 158. The ion exchanger of any one of embodiments 131 to 155, wherein the particulate metal titanate ion exchanger is substantially insoluble at a pH range of 1-13.

Embodiment 159. The ion exchanger of any one of embodiments 131 to 155, wherein the particulate metal titanate ion exchanger is substantially insoluble at physiological pH.

Embodiment 160. The ion exchanger of any one of embodiments 131 to 155, and 159, wherein the particulate metal titanate ion exchanger is substantially insoluble at stomach pH.

Embodiment 161. The ion exchanger of any one of embodiments 131 to 160, wherein the macroporous particulate titanate ion exchanger is substantially insoluble in one or more bodily fluids.

Embodiment 162. The ion exchanger of embodiment 161, wherein the one or more bodily fluids are selected from the group consisting of blood, urine, and gastrointestinal fluid.

Embodiment 163. The ion exchanger of any one of embodiments 131 to 162, wherein the macroporous particulate titanate ion exchanger has a distribution coefficient (K_{d}) for Pb²⁺ of between about 100,000 to about 2,500,000 milliliters per gram (ml/g).

Embodiment 164. The ion exchanger of embodiment 163, wherein the macroporous particulate titanate ion exchanger has a K_{d} for Pb²⁺ of about 967,800.

Embodiment 165. The ion exchanger of embodiment 163, wherein the macroporous particulate titanate ion exchanger has a K_{d} for Pb²⁺ of about 321,900.

Embodiment 166. The ion exchanger of any one of embodiments 131 to 165, wherein the macroporous particulate titanate ion exchanger comprises between 0.01% to 4.0% weight per weight (w/w) of the MHCA.

Embodiment 167. The ion exchanger of any one of embodiments 131 to 166, wherein m is about 0.28.

Embodiment 168. A method for manufacturing a tablet or capsule for oral administration, the tablet or capsule comprising a particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the particulate metal titanate ion exchanger exhibits a median particle size of greater than 3 microns (μm), the method comprising the steps of:
   (a) forming a reaction mixture comprising reactive sources of A, Ti, at least one multihydroxyl-containing complexing agent (MHCA), optionally M, optionally hydrogen peroxide, optionally a complexing agent (C), and water,
   (b) heating the reaction mixture for a period of time sufficient to form a particulate metal titanate ion exchanger,
   (c) optionally treating the metal titanate ion exchanger via acid extraction and/or ion exchange with alkali metals, alkaline earth metals, or mixtures thereof to form the particulate metal titanate ion exchanger with the desired composition, and
   (d) forming a capsule or tablet comprising the particulate metal titanate ion exchanger, wherein the reaction mixture comprises a composition expressed in terms of mole ratios of the oxides of:

p A₂O: a TiO₂:b MO_{q/2}:c H₂O₂:d MHCA:e C:f H₂O
wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000.

Embodiment 169. The method of embodiment 168, wherein the at least one MHCA is selected from the group consisting of a sugar alcohol, a sugar, and an aromatic compound.

Embodiment 170. The method of embodiment 168 or embodiment 169, wherein the at least one MHCA is d-sorbitol, mannitol, xylitol, catechol, fructose, or glucose.

Embodiment 171. The method of embodiments 168 to 170, wherein the source of Ti is Ti(OiPr)₄ and the reaction mixture is a solution.

Embodiment 172. The method of any one of embodiments 168 to 170, wherein the source of Ti is TiO₂ powder, including nano-sized TiO₂, or preformed spray dried TiO₂ spheres.

Embodiment 173. The method of embodiment 172, wherein the source of Ti further comprises Ti(OiPr)₄.

Embodiment 174. The method of any one of embodiments 168 to 173, wherein C is selected from the group consisting of citric acid, tartaric acid, EDTA, bipyridine, and any combination thereof.

Embodiment 175. The method of embodiment 174, wherein C is citric acid.

Embodiment 176. The method of any one of embodiments 168 to 175, wherein the hydrogen peroxide is 30 weight percent (wt. %) aqueous hydrogen peroxide.

Embodiment 177. The method of any one of embodiments 168 to 176, wherein the alkali base is potassium hydroxide.

Embodiment 178. The method of any one of embodiments 168 to 177, wherein the heating is at about 85° C. to about 225° C.

Embodiment 179. The method of any one of embodiments 168 to 178, wherein the period of time is between 0.5 days and 30 days.

Embodiment 180. The method of any one of embodiments 168 to 179, further comprising a step of treating the particulate metal titanate ion exchanger with an acid following step (b).

Embodiment 181. The method of embodiment 180, wherein the acid is selected from the group consisting of nitric acid, hydrochloric acid, perchloric acid, and sulfuric acid.

Embodiment 182. The method of embodiment 181, wherein the acid is nitric acid.

Embodiment 183. The method of any one of embodiments 180 to 182 wherein the treating the particulate metal titanate ion exchanger with an acid is at a pH of between 1 and 3 for at least 15 minutes.

Embodiment 184. The method of any one of embodiments 168 to 183, further comprising a step of sterilizing the particulate metal titanate ion exchanger.

Embodiment 185. The method of embodiment 184, wherein the sterilizing is by means of an autoclave.

Embodiment 186. The method of any one of embodiments 168 to 185, wherein the forming the capsule or tablet further comprises adding a binding agent.

Embodiment 187. The method of embodiment 186, wherein the binding agent is zirconia.

Embodiment 188. The method of any one of embodiments 168 to 187, wherein forming the capsule or tablet comprises annealing the particulate metal titanate ion exchanger.

Embodiment 189. The method of any one of embodiments 168 to 188, further comprising a step of spray drying the particulate metal titanate ion exchanger to form enlarged aggregates of the particulate metal titanate ion exchanger of the prior to forming the capsule or tablet.

Embodiment 190. The method of any one of embodiments 168 to 189, wherein A is potassium ion, hydronium ion, or a mixture thereof.

Embodiment 191. The method of any one of embodiments 168 to 190, wherein A is potassium ion.

Embodiment 192. The method of any one of embodiments 168 to 190, wherein A is hydronium ion.

Embodiment 193. The method of any one of embodiments 168 to 190, wherein A is a mixture of potassium and hydronium ions.

Embodiment 194. The method of any one of embodiments 168 to 193, wherein the particulate metal titanate ion exchanger is a polycrystalline aggregate metal titanate ion exchanger.

Embodiment 195. The method of any one of embodiments 168 to 194, wherein the particulate metal titanate ion exchanger is macroporous.

Embodiment 196. The method of any one of embodiments 168 to 195, wherein the particulate metal titanate ion exchanger has spherical morphology.

Embodiment 197. The method of any one of embodiments 168 to 195, wherein the particulate metal titanate ion exchanger has amorphous morphology.

Embodiment 198. The method of any one of embodiments 168 to 197, wherein the particulate metal titanate ion exchanger is a powder.

Embodiment 199. The method of any one of embodiments 168 to 198, wherein the median particle size is between 25 to 125 microns (μm).

Embodiment 200. The method of any one of embodiments 168 to 199, wherein less than 3% of the particles of the particulate metal titanate ion exchanger have a particle size of less than 3 microns (μm).

Embodiment 201. The method of embodiment 200, wherein less than 0.5% of the particles of the particulate metal titanate ion exchanger have a particle size less than 3 microns (μm).

Embodiment 202. The method of any one of embodiments 168 to 201, wherein the particulate metal titanate ion exchanger has a particle size distribution d₁₀ value of between about 5 microns (μm) and about 70 μm.

Embodiment 203. The method of embodiment 202, wherein the particulate metal titanate ion exchanger has a particle size distribution d₁₀ value of between about 12 μm and about 18 μm.

Embodiment 204. The method of embodiment 203, wherein the particulate metal titanate ion exchanger has a particle size distribution d₁₀ value of about 15 μm.

Embodiment 205. The method of embodiment 202, wherein the particulate metal titanate ion exchanger has a particle size distribution d₁₀ value of between about 42 μm and about 48 μm.

Embodiment 206. The method of embodiment 205, wherein the particulate metal titanate ion exchanger has a particle size distribution d₁₀ value of about 45 μm.

Embodiment 207. The method of embodiment 202, wherein the particulate metal titanate ion exchanger has a particle size distribution d₁₀ value of between about 51 μm and about 57 μm.

Embodiment 208. The method of embodiment 207, wherein the particulate metal titanate ion exchanger has a particle size distribution d₁₀ value of about 54 μm.

Embodiment 209. The method of any one of embodiments 168 to 208, wherein the particulate metal titanate ion exchanger has a particle size distribution d₅₀ value of between about 25 microns (μm) and about 125 μm.

Embodiment 210. The method of embodiment 209, wherein the particulate metal titanate ion exchanger has a particle size distribution d₅₀ value of between about 48 μm and about 54 μm.

Embodiment 211. The method of embodiment 210, wherein the particulate metal titanate ion exchanger has a particle size distribution d₅₀ value of about 51 μm.

Embodiment 212. The method of embodiment 209, wherein the particulate metal titanate ion exchanger has a particle size distribution d₅₀ value of between about 71 μm and about 77 μm.

Embodiment 213. The method of embodiment 212, wherein the particulate metal titanate ion exchanger has a particle size distribution d₅₀ value of about 74 μm.

Embodiment 214. The method of embodiment 209, wherein the particulate metal titanate ion exchanger has a particle size distribution d₅₀ value of between about 89 μm and about 95 μm.

Embodiment 215. The method of embodiment 214, wherein the particulate metal titanate ion exchanger has a particle size distribution d₅₀ value of about 92 μm.

Embodiment 216. The method of any one of embodiments 168 to 215, wherein the particulate metal titanate ion exchanger has a particle size distribution d₉₀ value of between about 55 microns (μm) and about 185 μm.

Embodiment 217. The method of embodiment 216, wherein the particulate metal titanate ion exchanger has a particle size distribution d₉₀ value of between about 99 μm and about 105 μm.

Embodiment 218. The method of embodiment 217, wherein the particulate metal titanate ion exchanger has a particle size distribution d₉₀ value of about 102 μm.

Embodiment 219. The method of embodiment 216, wherein the particulate metal titanate ion exchanger has a particle size distribution d₉₀ value of between about 137 μm and about 143 μm.

Embodiment 220. The method of embodiment 219, wherein the particulate metal titanate ion exchanger has a particle size distribution d₉₀ value of about 140 μm.

Embodiment 221. The method of embodiment 216, wherein the particulate metal titanate ion exchanger has a particle size distribution d₉₀ value of between about 156 μm and about 162 μm.

Embodiment 222. The method of embodiment 221, wherein the particulate metal titanate ion exchanger has a particle size distribution d₉₀ value of about 159 μm.

Embodiment 223. The method of any one of embodiments 168 to 222, wherein the particulate metal titanate ion exchanger is stable in a liquid environment at a pH of 1-2.

Embodiment 224. The method of any one of embodiments 168 to 223, wherein the particulate metal titanate ion exchanger has a Brunauer-Emmett-Teller (BET) surface area of greater than 150 square meters per gram (m²/g).

Embodiment 225. The method of embodiment 224, wherein the particulate metal titanate ion exchanger has a BET surface area of about 197 m²/g.

Embodiment 226. The method of any one of embodiments 168 to 225, wherein the particulate metal titanate ion exchanger has a Brunauer-Emmett-Teller (BET) surface area of greater than 200 square meters per gram (m²/g).

Embodiment 227. The method of embodiment 226, wherein the particulate metal titanate ion exchanger has a BET surface area of about 203 m²/g.

Embodiment 228. The method of any one of embodiments 168 to 225, wherein the particulate metal titanate ion exchanger has a Brunauer-Emmett-Teller (BET) surface area of greater than 230 square meters per gram (m²/g).

Embodiment 229. The method of embodiment 228, wherein the particulate metal titanate ion exchanger has a BET surface area of about 236 m²/g.

Embodiment 230. The method of any one of embodiments 168 to 229, wherein the particulate metal titanate ion exchanger is substantially insoluble at a pH range of 1-7.

Embodiment 231. The method of any one of embodiments 168 to 229, wherein the particulate metal titanate ion exchanger is substantially insoluble at a pH range of 7-13.

Embodiment 232. The method of any one of embodiments 168 to 229, wherein the particulate metal titanate ion exchanger is substantially insoluble at a pH range of 1-13.

Embodiment 233. The method of any one of embodiments 168 to 229, wherein the particulate metal titanate ion exchanger is substantially insoluble at physiological pH.

Embodiment 234. The method of any one of embodiments 168 to 229, and 233, wherein the particulate metal titanate ion exchanger is substantially insoluble at stomach pH.

Embodiment 235. The method of any one of embodiments 168 to 234, wherein the particulate metal titanate ion exchanger is substantially insoluble in one or more bodily fluids.

Embodiment 236. The method of embodiment 235, wherein the one or more bodily fluids are selected from the group consisting of blood, urine, and gastrointestinal fluid.

Embodiment 237. The method of any one of embodiments 168 to 236, wherein the particulate metal titanate ion exchanger has a distribution coefficient (K_{d}) for Pb²⁺ of between about 50,000 to about 5,500,000 milliliters per gram (mL/g) in solution.

Embodiment 238. The method of embodiment 237, wherein the particulate metal titanate ion exchanger has a K_{d} for Pb²⁺ of between about 100,000 to about 2,500,000 milliliters per gram (mL/g).

Embodiment 239. The method of embodiment 238, wherein the particulate metal titanate ion exchanger has a K_{d} for Pb²⁺ of about 967,800.

Embodiment 240. The method of embodiment 238, wherein the particulate metal titanate ion exchanger has a K_{d} for Pb²⁺ of about 809,500.

Embodiment 241. The method of embodiment 238, wherein the particulate metal titanate ion exchanger has a K_{d} for Pb²⁺ of about 495,800.

Embodiment 242. The method of embodiment 238, wherein the particulate metal titanate ion exchanger has a K_{d} for Pb²⁺ of about 321,900.

Embodiment 243. The method of any one of embodiments 168 to 242, wherein the particulate metal titanate ion exchanger comprises between 0.01% to 4.0% weight per weight (w/w) of the at least one MHCA.

Embodiment 244. The method of embodiment 243, wherein the particulate metal titanate ion exchanger comprises between 0.01% to 0.6% w/w of the at least one MHCA.

Embodiment 245. The method of any one of embodiments 168 to 244, wherein x is 1 and y is 0.

Embodiment 246. The method of any one of embodiments 168 to 245, wherein m is between 0.10 to 0.50.

Embodiment 247. The method of embodiment 246, wherein m is about 0.40.

Embodiment 248. The method of embodiment 246, wherein m is about 0.30.

Embodiment 249. The method of embodiment 246, wherein m is about 0.28.

Embodiment 250. The method of any one of embodiments 168 to 249, wherein the x-ray diffraction (XRD) pattern of the particulate metal titanate ion exchanger has a characteristic diffraction line with a d-spacing of 3.00 to 3.11 Å.

Embodiment 251. The method of any one of embodiments 168 to 250, wherein the particulate metal titanate ion exchanger has an x-ray diffraction (XRD) pattern having characteristic diffraction lines within the ranges provided below in Table A or Table B:

TABLE A

| 2Θ | d(Å) | I/I₀ |
|---|---|---|
| 11.11-10.78 | 7.96-8.2 | w-m |
| 25.43-24.03 | 3.50-3.70 | w-m |
| 29.55-28.68 | 3.02-3.11 | vs |
| 33.93-33.15 | 2.64-2.70 | m-s |
| 43.36-42.51 | 2.085-2.125 | w-m |
| 48.38-47.31 | 1.88-1.92 | m-s |
| 60.46-59.18 | 1.53-1.56 | w-m |
| 67.03-65.44 | 1.395-1.425 | w-m |

TABLE B

| 2Θ | d(Å) | I/I₀ |
|---|---|---|
| 11.47-10.95 | 7.71-8.07 | w-s |
| 24.37-23.97 | 3.65-3.71 | m-s |
| 29.76-28.78 | 3.00-3.10 | vs |
| 33.80-33.15 | 2.65-2.70 | w-m |
| 43.47-42.51 | 2.08-2.125 | m-s |
| 48.51-47.49 | 1.875-1.913 | m-vs |
| 66.93-65.55 | 1.397-1.423 | w-m. |

Embodiment 252. The method of any one of embodiments 168 to 249, wherein the x-ray diffraction (XRD) pattern of the particulate metal titanate ion exchanger has a characteristic diffraction line with a d-spacing of 3.00 to 3.10 Å.

Embodiment 253. The method of any one of embodiments 168 to 249, and 252, wherein the particulate metal titanate ion exchanger has an x-ray diffraction (XRD) pattern having characteristic diffraction lines within the ranges provided below in Table C or Table D:

TABLE C

| 2Θ | d(Å) | I/I₀ |
|---|---|---|
| 11.87-10.89 | 7.45-8.12 | w-s |
| 24.50-23.71 | 3.63-3.75 | w-m |
| 29.76-28.87 | 3.00-3.09 | vs |
| 32.06-30.81 | 2.79-2.90 | w-s |
| 33.93-33.15 | 2.64-2.70 | m-s |
| 43.26-42.61 | 2.09-2.12 | w-s |
| 48.24-47.44 | 1.885-1.915 | w-s |
| 60.03-58.77 | 1.54-1.57 | w-m |
| 66.77-65.71 | 1.40-1.42 | w-m |

TABLE D

| 2Θ | d(Å) | I/I₀ |
|---|---|---|
| 11.76-10.82 | 7.52-8.17 | w-s |
| 24.64-23.58 | 3.61-3.77 | w-m |
| 29.76-28.78 | 3.00-3.10 | vs |
| 32.05-30.38 | 2.79-2.94 | w-s |
| 34.06-33.15 | 2.63-2.70 | w-m |
| 43.36-42.51 | 2.085-2.125 | m-vs |
| 48.51-47.44 | 1.875-1.915 | m-vs |
| 60.24-58.76 | 1.535-1.57 | w-m |
| 66.76-65.70 | 1.40-1.42 | w-m |

Embodiment 254. A method for selectively removing Pb²⁺ toxins from gastrointestinal fluid, the process comprising contacting the fluid containing the toxins with a particulate metal titanate ion exchanger, resulting in an ion exchanged ion exchanger and thereby removing the Pb²⁺ toxins from the fluid, the particulate metal titanate ion exchanger having an empirical formula on an anhydrous basis of:

AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the metal titanate ion exchanger having been synthesized in the presence of at least one multihydroxyl-containing complexing agent (MHCA), wherein the particulate metal titanate ion exchanger exhibits a median particle size of greater than 3 microns (μm), and wherein the particulate metal titanate ion exchanger minimally disrupts the levels of any one or more ions selected from Na⁺, Mg²⁺, K⁺, and Ca²⁺ from the fluid.

Embodiment 255. The method of embodiment 254, wherein the particulate metal titanate ion exchanger is an acid-treated particulate metal titanate ion exchanger.

Embodiment 256. The method of embodiment 254 or embodiment 255, wherein A is potassium ion, hydronium ion, or a mixture thereof.

Embodiment 257. The method of any one of embodiments 254 to 256, wherein A is potassium ion.

Embodiment 258. The method of any one of embodiments 254 to 256, wherein A is hydronium ion.

Embodiment 259. The method of any one of embodiments 254 to 256, wherein A is a mixture of potassium and hydronium ions.

Embodiment 260. The method of any one of embodiments 254 to 259, wherein the particulate metal titanate ion exchanger is a polycrystalline aggregate metal titanate ion exchanger.

Embodiment 261. The method of any one of embodiments 254 to 260, wherein the particulate metal titanate ion exchanger is macroporous.

Embodiment 262. The method of any one of embodiments 254 to 261, wherein the particulate metal titanate ion exchanger has a spherical morphology.

Embodiment 263. The method of any one of embodiments 254 to 261, wherein the particulate metal titanate ion exchanger has an amorphous morphology.

Embodiment 264. The method of any one of embodiments 254 to 263, wherein the particulate metal titanate ion exchanger is a powder.

Embodiment 265. The method of any one of embodiments 254 to 264, wherein the median particle size is between 25 to 125 microns (μm).

Embodiment 266. The method of any one of embodiments 254 to 265, wherein less than 3% of the particles of the particulate metal titanate ion exchanger exhibit a particle size less than 3 microns (μm).

Embodiment 267. The method of embodiment 266, wherein less than 0.5% of the particles of the particulate metal titanate ion exchanger exhibit a particle size less than 3 microns (μm).

Embodiment 268. The method of any one of embodiments 254 to 267, wherein the particulate metal titanate ion exchanger has a particle size distribution d₁₀ value of between about 5 microns (μm) and about 70 μm.

Embodiment 269. The method of embodiment 268, wherein the particulate metal titanate ion exchanger has a particle size distribution d₁₀ value of between about 12 μm and about 18 μm.

Embodiment 270. The method of embodiment 269, wherein the particulate metal titanate ion exchanger has a particle size distribution d₁₀ value of about 15 μm.

Embodiment 271. The method of embodiment 268, wherein the particulate metal titanate ion exchanger has a particle size distribution d₁₀ value of between about 42 μm and about 48 μm.

Embodiment 272. The method of embodiment 271, wherein the particulate metal titanate ion exchanger has a particle size distribution d₁₀ value of about 45 μm.

Embodiment 273. The method of embodiment 268, wherein the particulate metal titanate ion exchanger has a particle size distribution do value of between about 51 μm and about 57 μm.

Embodiment 274. The method of embodiment 273, wherein the particulate metal titanate ion exchanger has a particle size distribution d₁₀ value of about 54 μm.

Embodiment 275. The method of any one of embodiments 254 to 274, wherein the particulate metal titanate ion exchanger has a particle size distribution d₅₀ value of between about 25 microns (μm) and about 125 μm.

Embodiment 276. The method of embodiment 275, wherein the particulate metal titanate ion exchanger has a particle size distribution d₅₀ value of between about 48 μm and about 54 μm.

Embodiment 277. The method of embodiment 276, wherein the particulate metal titanate ion exchanger has a particle size distribution d₅₀ value of about 51 μm.

Embodiment 278. The method of embodiment 275, wherein the particulate metal titanate ion exchanger has a particle size distribution d₅₀ value of between about 71 μm and about 77 μm.

Embodiment 279. The method of embodiment 278, wherein the particulate metal titanate ion exchanger has a particle size distribution d₅₀ value of about 74 μm.

Embodiment 280. The method of embodiment 275, wherein the particulate metal titanate ion exchanger has a particle size distribution d₅₀ value of between about 89 μm and about 95 μm.

Embodiment 281. The method of embodiment 280, wherein the particulate metal titanate ion exchanger has a particle size distribution d₅₀ value of about 92 μm.

Embodiment 282. The method of any one of embodiments 254 to 281, wherein the particulate metal titanate ion exchanger has a particle size distribution d₉₀ value of between about 55 microns (μm) and about 185 μm.

Embodiment 283. The method of embodiment 282, wherein the particulate metal titanate ion exchanger has a particle size distribution d₉₀ value of between about 99 μm and about 105 μm.

Embodiment 284. The method of embodiment 283, wherein the particulate metal titanate ion exchanger has a particle size distribution d₉₀ value of about 102 μm.

Embodiment 285. The method of embodiment 282, wherein the particulate metal titanate ion exchanger has a particle size distribution d₉₀ value of between about 137 μm and about 143 μm.

Embodiment 286. The method of embodiment 285, wherein the particulate metal titanate ion exchanger has a particle size distribution d₉₀ value of about 140 μm.

Embodiment 287. The method of embodiment 282, wherein the particulate metal titanate ion exchanger has a particle size distribution d₉₀ value of between about 156 μm and about 162 μm.

Embodiment 288. The method of embodiment 287, wherein the particulate metal titanate ion exchanger has a particle size distribution d₉₀ value of about 159 μm.

Embodiment 289. The method of any one of embodiments 254 to 288, wherein the particulate metal titanate ion exchanger is stable in a liquid environment at a pH of 1-2.

Embodiment 290. The method of any one of embodiments 254 to 289, wherein the particulate metal titanate ion exchanger has a Brunauer-Emmett-Teller (BET) surface area of greater than 150 square meters per gram (m²/g).

Embodiment 291. The method of embodiment 290, wherein the particulate metal titanate ion exchanger has a BET surface area of about 197 m²/g.

Embodiment 292. The method of any one of embodiments 254 to 289, wherein the particulate metal titanate ion exchanger has a Brunauer-Emmett-Teller (BET) surface area of greater than 200 square meters per gram (m²/g).

Embodiment 293. The method of embodiment 292, wherein the particulate metal titanate ion exchanger has a BET surface area of about 203 m²/g.

Embodiment 294. The method of any one of embodiments 254 to 289, wherein the particulate metal titanate ion exchanger has a Brunauer-Emmett-Teller (BET) surface area of greater than 230 square meters per gram (m²/g).

Embodiment 295. The method of embodiment 294, wherein the particulate metal titanate ion exchanger has a BET surface area of about 236 m²/g.

Embodiment 296. The method of any one of embodiments 254 to 295, wherein the particulate metal titanate ion exchanger is substantially insoluble at a pH range of 1-7.

Embodiment 297. The method of any one of embodiments 254 to 295, wherein the particulate metal titanate ion exchanger is substantially insoluble at a pH range of 7-13.

Embodiment 298. The method of any one of embodiments 254 to 295, wherein the particulate metal titanate ion exchanger is substantially insoluble at a pH range of 1-13.

Embodiment 299. The method of any one of embodiments 254 to 298, wherein the particulate metal titanate ion exchanger is substantially insoluble in the fluid.

Embodiment 300. The method of any one of embodiments 254 to 299, wherein the particulate metal titanate ion exchanger has a distribution coefficient (K_{d}) for Pb²⁺ of between about 50,000 to about 5,500,000 milliliters per gram (ml/g).

Embodiment 301. The method of embodiment 300, wherein the particulate metal titanate ion exchanger has a K_{d} for Pb²⁺ of between about 100,000 to about 2,500,000 mL/g.

Embodiment 302. The method of embodiment 301, wherein the particulate metal titanate ion exchanger has a K_{d} for Pb²⁺ of about 967,800.

Embodiment 303. The method of embodiment 301, wherein the particulate metal titanate ion exchanger has a K_{d} for Pb²⁺ of about 809,500.

Embodiment 304. The method of embodiment 301, wherein the particulate metal titanate ion exchanger has a K_{d} for Pb²⁺ of about 495,800.

Embodiment 305. The method of embodiment 301, wherein the particulate metal titanate ion exchanger has a K_{d} for Pb²⁺ of about 321,900.

Embodiment 306. The method of any one of embodiments 254 to 305, wherein the at least one MHCA is selected from the group consisting of a sugar alcohol, a sugar, an aromatic compound, and any combination thereof.

Embodiment 307. The method of embodiment 306, wherein the at least one MHCA is a sugar alcohol.

Embodiment 308. The method of embodiment 307, wherein the sugar alcohol is selected from the group consisting of d-sorbitol, mannitol, and xylitol.

Embodiment 309. The method of any one of embodiments 306 to 308, wherein the at least one MHCA is d-sorbitol.

Embodiment 310. The method of embodiment 306, wherein the at least one MHCA is a sugar.

Embodiment 311. The method of embodiment 310, wherein the sugar is glucose or fructose.

Embodiment 312. The method of embodiment 306, wherein the at least one MHCA is an aromatic compound.

Embodiment 313. The method of embodiment 312, wherein the aromatic compound is catechol.

Embodiment 314. The method of any one of embodiments 254 to 313, wherein the particulate metal titanate ion exchanger comprises between 0.01% to 4.0% weight per weight (w/w) of the at least one MHCA.

Embodiment 315. The method of embodiment 314, wherein the particulate metal titanate ion exchanger comprises between 0.01% to 0.6% w/w of the at least one MHCA.

Embodiment 316. The method of any one of embodiments 254 to 315, wherein x is 1 and y is 0.

Embodiment 317. The method of any one of embodiments 254 to 316, wherein m is between 0.10 to 0.50.

Embodiment 318. The method of embodiment 317, wherein m is about 0.40.

Embodiment 319. The method of embodiment 317, wherein m is about 0.30.

Embodiment 320. The method of embodiment 317, wherein m is about 0.28.

Embodiment 321. The method of any one of embodiments 254 to 320, wherein the x-ray diffraction (XRD) pattern of the particulate metal titanate ion exchanger has a characteristic diffraction line with a d-spacing of 3.00 to 3.11 Å.

Embodiment 322. The method of any one of embodiments 254 to 321, wherein the particulate metal titanate ion exchanger has an x-ray diffraction (XRD) pattern having characteristic diffraction lines within the ranges provided below in Table A or Table B:

TABLE A

| 2Θ | d(Å) | I/I₀ |
|---|---|---|
| 11.11-10.78 | 7.96-8.2 | w-m |
| 25.43-24.03 | 3.50-3.70 | w-m |
| 29.55-28.68 | 3.02-3.11 | vs |
| 33.93-33.15 | 2.64-2.70 | m-s |
| 43.36-42.51 | 2.085-2.125 | w-m |
| 48.38-47.31 | 1.88-1.92 | m-s |
| 60.46-59.18 | 1.53-1.56 | w-m |
| 67.03-65.44 | 1.395-1.425 | w-m |

TABLE B

| 2Θ | d(Å) | I/I₀ |
|---|---|---|
| 11.47-10.95 | 7.71-8.07 | w-s |
| 24.37-23.97 | 3.65-3.71 | m-s |
| 29.76-28.78 | 3.00-3.10 | vs |
| 33.80-33.15 | 2.65-2.70 | w-m |
| 43.47-42.51 | 2.08-2.125 | m-s |
| 48.51-47.49 | 1.875-1.913 | m-vs |
| 66.93-65.55 | 1.397-1.423 | w-m. |

Embodiment 323. The method of any one of embodiments 254 to 320, wherein the x-ray diffraction (XRD) pattern of the particulate metal titanate ion exchanger has a characteristic diffraction line with a d-spacing of 3.00 to 3.10 Å.

Embodiment 324. The method of any one of embodiments 254 to 320, and 322, wherein the particulate metal titanate ion exchanger has an x-ray diffraction (XRD) pattern having characteristic diffraction lines within the ranges provided below in Table C or Table D:

TABLE C

| 2Θ | d(Å) | I/I₀ |
|---|---|---|
| 11.87-10.89 | 7.45-8.12 | w-s |
| 24.50-23.71 | 3.63-3.75 | w-m |
| 29.76-28.87 | 3.00-3.09 | vs |
| 32.06-30.81 | 2.79-2.90 | w-s |
| 33.93-33.15 | 2.64-2.70 | m-s |
| 43.26-42.61 | 2.09-2.12 | w-s |
| 48.24-47.44 | 1.885-1.915 | w-s |
| 60.03-58.77 | 1.54-1.57 | w-m |
| 66.77-65.71 | 1.40-1.42 | w-m |

TABLE D

| 2Θ | d(Å) | I/I₀ |
|---|---|---|
| 11.76-10.82 | 7.52-8.17 | w-s |
| 24.64-23.58 | 3.61-3.77 | w-m |
| 29.76-28.78 | 3.00-3.10 | vs |
| 32.05-30.38 | 2.79-2.94 | w-s |
| 34.06-33.15 | 2.63-2.70 | w-m |
| 43.36-42.51 | 2.085-2.125 | m-vs |
| 48.51-47.44 | 1.875-1.915 | m-vs |
| 60.24-58.76 | 1.535-1.57 | w-m |
| 66.76-65.70 | 1.40-1.42 | w-m |

Embodiment 325. The method of any one of embodiments 254 to 324, wherein the particulate metal titanate ion exchanger minimally disrupts the levels of any one or more ions selected from Na⁺, Mg²⁺, K⁺, and Ca^{2+.}

Embodiment 326. The method of embodiment 325, wherein the particulate metal titanate ion exchanger reduces the levels of Nations by 12 mg/dL or less.

Embodiment 327. The method of embodiment 325, wherein the particulate metal titanate ion exchanger reduces the levels of K⁺ ions by 3.0 mg/dL or less.

Embodiment 328. The method of embodiment 325, wherein the particulate metal titanate ion exchanger reduces the levels of Mg²⁺ ions by 0.6 mg/dL or less.

Embodiment 329. The method of embodiment 325, wherein the particulate metal titanate ion exchanger reduces the levels of Ca²⁺ ions by 1.0 mg/dL or less.

Embodiment 330. The method of embodiment 254 to 324, wherein the particulate metal titanate ion exchanger does not substantially reduce the levels of any one or more ions selected from Na⁺, Mg²⁺, K⁺, and Ca^{2+.}

Embodiment 331. The method of any one of embodiments 254 to 330, wherein the one or more ions are Na⁺, Mg²⁺, K⁺, and Ca^{2+.}

Embodiment 332. The method of any one of embodiments 254 to 331, wherein the levels of the any one or more ions selected from Na⁺, Mg²⁺, K⁺, and Ca²⁺ are measured by inductively coupled plasma (ICP) elemental analysis of the fluid.

Embodiment 333. The method of any one of embodiments 254 to 332, wherein unbound Pb²⁺ toxins are not detectable in the fluid after the contacting as measured by inductively coupled plasma (ICP) elemental analysis of the fluid.

Embodiment 334. The method of any one of embodiments 254 to 333, wherein the Pb²⁺ toxins are sequestered within the ion exchanged ion exchanger after the contacting.

Embodiment 335. The method of any one of embodiments 254 to 334, wherein the selectively removing is an intracorporeal process.

### EXAMPLES

The x-ray patterns presented in the following examples were obtained using standard x-ray powder diffraction techniques. The radiation source was a high-intensity, x-ray tube operated at 45 kV and 35 mA. The diffraction pattern from the copper K-alpha radiation was obtained by appropriate computer-based techniques. Flat compressed powder samples were continuously scanned at 2° to at least 56° (20). Interplanar spacings (d) in Angstrom units were obtained from the position of the diffraction peaks expressed as 0 where 0 is the Bragg angle as observed from digitized data. Intensities were determined from the integrated area of diffraction peaks after subtracting background, “Iₒ” being the intensity of the strongest line or peak, and “I” being the intensity of each of the other peaks.

As will be understood by those skilled in the art, without being limited by theory, the determination of the parameter 20 is subject to both human and mechanical error, which in combination can impose an uncertainty of about +0.4° on each reported value of 20. This uncertainty is also manifested in the reported values of the d-spacings, which are calculated from the 20 values. This imprecision is general throughout the art and is not sufficient to preclude the differentiation of the present crystalline materials from each other and from the compositions of the prior art. In the x-ray patterns reported, the relative intensities of the d-spacings are indicated by the notations vs, s, m, and w which represent very strong, strong, medium, and weak, respectively. In terms of 100×I/Iₒ, the above designations are defined as:

$$w > 0 - 15 ; m > 15 - 60 : s > 60 - 80 and vs > 80 - 100.$$

In certain instances, the purity of a synthesized product may be assessed with reference to its x-ray powder diffraction pattern. Thus, for example, if a sample is stated to be pure, it is intended only that the x-ray pattern of the sample is free of lines attributable to crystalline impurities, not that there are no amorphous materials present.

Additionally, while elemental analysis can be used to determine the metals stoichiometry, the elements oxygen and hydrogen and water are not determined by this analysis method. Oxygen stoichiometry is inferred by balancing the charges on the metals, thus the metal titanate ion exchanger compositions of the present disclosure are described in their anhydrous state.

### Examples 1-15: Synthesis of Metal Titanates from Solution

Examples 1-15 provide examples of the synthesis of Metal Titanate ion exchangers of the present disclosure from homogenous solution. In an aspect, a process uses three different complexing agents, hydrogen peroxide to provide initial dissolution of Ti in acidic solution, a second complexing agent such as citric acid to help Ti and the M metals remain in solution as the pH is increased to about 10, and an MHCA agent such as d-sorbitol that will stabilize Ti and the M metals in homogenous solution at very high pHs appropriate for the synthesis of the metal titanate ion exchangers. Within each reaction mixture it is demonstrated that structure often changes with reaction conditions, choice of alkali, perturbations with alkali, and ion exchange. Examples that include more than one X-ray diffraction pattern yield more than one crystal structure from the described reaction mixture, depending on reaction conditions elicited in the given example, showing the diversity of structural outcomes from this novel synthetic approach. Results include large polycrystalline aggregate morphologies in the product, which are further addressed in Examples 26 and 27.

### Examples 1A & 1B

A Teflon beaker containing 90.00 g deionized water is fitted with an overhead mixer. With vigorous stirring, 21.11 g H₂O₂ (30 wt. %), 17.89 g citric acid and 16.96 g d-sorbitol are added and dissolved. Then 13.35 g Ti(OiPr)₄ (16.7% Ti) is added fast dropwise forming a dark red-orange solution after stirring for 2 minutes post addition. Separately, 38.00 g NaOH (98%) is dissolved in 75.00 g deionized water and allowed to stir and cool. This NaOH solution is added fast dropwise with vigorous stirring, forming a clear, nearly colorless solution after going through a series of color changes. The highly basic clear solution is distributed among 4 Teflon-lined reaction vessels and digested quiescently at 175 and 190° C. for 49 and 166 hr at autogenous pressures. Solid products are isolated by centrifugation, washed with deionized water, and dried at room temperature. Powder X-ray Diffraction (PXRD) is used to characterize the products. Characteristic diffraction lines for the Example 1A (175° C./49 hr) and Example 1B (190° C./166 hr) products are provided in Table 1.

TABLE 1

| Example 1A | | | Example 1B | | |
|---|---|---|---|---|---|
| 2-Θ | d(Å) | I/I₀ % | 2-Θ | d(Å) | I/I₀ % |
| 9.06 | 9.75 | vs (br) | 9.70 | 9.11 | vs (br) |
| 24.32 | 3.66 | m (br) | 24.20 | 3.68 | m (br) |
| 28.14 | 3.17 | m (br) | 28.54 | 3.12 | vs (br) |
| 48.22 | 1.89 | s (br) | 48.34 | 1.88 | s (br) |

### Example 2

A Teflon beaker containing 90.00 g deionized water is fitted with an overhead mixer. With vigorous stirring, 21.11 g H₂O₂ (30 wt. %), 17.89 g citric acid, and 16.96 g d-sorbitol are added resulting in a clear colorless solution. Then 13.35 g Ti(OiPr)₄ (16.7% Ti) is added fast dropwise to form a dark orange-red solution. Separately, 59.97 g KOH (87.1%) is dissolved in 100.00 g deionized water and allowed to stir and cool. The KOH solution is added fast dropwise with vigorous stirring forming a clear nearly colorless solution with a slight yellow tint after going through a series of color changes. The highly basic clear solution is distributed among 4 Teflon-lined reaction vessels and digested quiescently at 175 and 190° C. for 49 and 166 hr at autogenous pressures. Solid products are isolated by centrifugation, washed with deionized water, and dried at room temperature. PXRD is used to characterize the products, and characteristic diffraction lines for Example 2 (190° C./49 hr) are provided in Table 2.

TABLE 2

| 2-Θ | d(Å) | I/I₀ % |
|---|---|---|
| 10.44 | 8.47 | vs |
| 23.91 | 3.72 | w |
| 28.18 | 3.16 | vs |
| 47.66 | 1.91 | m |
| 48.75 | 1.87 | w |

### Examples 3A & 3B

A 3-liter polypropylene beaker is charged with 450.00 g deionized water and placed under an overhead mixer. With vigorous mixing, 158.71 g H₂O₂ (30 wt. %), 67.24 g citric acid, and 63.76 g d-sorbitol are added and dissolved. Separately, 171.43 g NaOH (98%) is dissolved in 250.0 g deionized water and allowed to stir and cool. Then, 100.32 g Ti(OiPr)₄ (16.7% Ti) is added to the 3-liter beaker, forming an orange-red solution with some precipitate that dissolved with stirring. The NaOH solution is added in 3 aliquots, and the reaction mixture is allowed to stir and cool after each aliquot is added. The final reaction mixture is a highly basic, slightly yellow solution. The reaction mixture is placed in a Teflon-lined 2 L Parr reactor and digested quiescently at 175° C. for 160 hr at autogenous pressure. The solid product is isolated by centrifugation, washed with deionized water, and dried at room temperature. PXRD is used to characterize the product. Characteristic diffraction lines for the Example 3A product are shown in Table 3 below. Elemental analysis via ICP yields the empirical metals composition Na_{0.36}Ti_{1.00} for the Example 3A material.

A 2.0 g portion of the product is ion-exchanged using 0.5 M Mg(NO₃)₂ solution (100 mL) at room temperature. The exchange is carried out 3 times before washing the product with deionized water and drying in air. The Example 3B product is characterized by PXRD, and characteristic diffraction lines are provided in Table 3. The Mg²⁺ exchanged product exhibits a different XRD pattern and has higher crystallinity than the parent.

TABLE 3

| Example 3A | | | Example 3B, Mg²⁺ IX | | |
|---|---|---|---|---|---|
| 2-Θ | d(Å) | I/I₀ % | 2-Θ | d(Å) | I/I₀ % |
| 9.02 | 9.80 | vs | 7.86 | 11.24 | vs |
| 24.22 | 3.67 | m | 15.76 | 5.62 | m |
| 28.68 | 3.11 | m | 17.90 | 4.95 | w |
| 48.26 | 1.88 | m | 24.22 | 3.67 | w |
| | | | 26.38 | 3.38 | w |
| | | | 48.44 | 1.85 | m |

### Examples 4A & 4B

This example provides an example of an alteration of the metal titanate structure via perturbation of what is similar to the Example 2 solution reaction mixture with LiCl. A Teflon beaker is charged with 90.00 g deionized water and placed under an overhead mixer. With vigorous stirring, 21.11 g H₂O₂ (30 wt. %), 17.89 g citric acid, and 16.96 g d-sorbitol are added and dissolved. Next, 13.35 g Ti(OiPr)₄ (16.7% Ti) is added fast dropwise forming a red-orange solution within minutes after the addition is complete. Separately, 59.99 g KOH (87.1%) is dissolved in 77.17 g deionized water and allowed to stir and cool. The KOH solution is added fast dropwise to the reaction mixture resulting in a clear, colorless solution. Separately, 3.95 g LiCl is dissolved in 12.00 g deionized water. This solution is added dropwise while vigorously stirring the reaction mixture, which remains a clear, colorless solution. The highly basic, clear solution is distributed among 8 Teflon-lined reaction vessels and digested quiescently at 125, 150, 175 and 190° C. for 52 and 168 hr at autogenous pressures. Solid products are isolated by centrifugation, washed with deionized water, and dried at room temperature. PXRD is used to characterize the products. Characteristic diffraction lines for the Example 4A (150° C./52 hr) and Example 4B (190° C./168 hr) products are provided in Table 4.

TABLE 4

| Example 4A | | | Example 4B | | |
|---|---|---|---|---|---|
| 2-Θ | d(Å) | I/I₀ % | 2-Θ | d(Å) | I/I₀ % |
| 10.68 | 8.28 | vs | 11.10 | 7.96 | vs |
| 23.94 | 3.71 | w | 23.98 | 3.71 | w |
| 28.44 | 3.14 | s | 28.82 | 3.10 | vs |
| 31.75 | 2.82 | w | 32.15 | 2.78 | w |
| 36.92 | 2.43 | s | 37.74 | 2.38 | m |
| 38.54 | 2.33 | w | 43.38 | 2.08 | s |
| 44.52 | 2.03 | m | 47.60 | 1.91 | m |
| 47.62 | 1.91 | m | | | |
| 49.00 | 1.86 | w | | | |
| 54.96 | 1.67 | m | | | |

### Examples 5A & 5B

A Teflon beaker is charged with 90.00 g deionized water and placed under an overhead mixer. With vigorous stirring, 21.11 g H₂O₂ (30 wt. %), 17.89 g citric acid, and 16.96 g d-sorbitol are added and dissolved. Next, 3.76 g Fe(NO₃)₃*9H₂O solid is added slowly forming a yellow solution. This is followed by fast dropwise addition of 10.68 g Ti(OiPr)₄ (16.7% Ti), yielding a dark red-orange solution. Separately, 38.01 g NaOH (98%) is dissolved in 77.17 g deionized water and allowed to stir and cool. The NaOH solution is added fast dropwise, during which the reaction mixture changes color to a brownish solution. The highly basic clear solution is distributed among 8 Teflon-lined reaction vessels and digested quiescently at 125, 150, 175 and 190° C. for 55 and 171 hr at autogenous pressures. Solid products are isolated by centrifugation, washed with deionized water, and dried at room temperature. PXRD is used to characterize the products. Characteristic diffraction lines for the Example 5A (150° C./55 hr) and Example 5B (190° C./171 hr) products are provided in Table 5. Elemental analysis via ICP yields the empirical composition with metals stoichiometry Na_{0.25}Fe_{0.20}Ti_{0.80} for the Example 5B material.

TABLE 5

| | | |
|---|---|---|
| 2-Φ | d(Å) | I/I₀% |

Example 5A

| | | |
|---|---|---|
| 8.02 | 11.02 | vs |
| 24.42 | 3.64 | w |
| 28.04 | 3.18 | s |
| 33.72 | 2.66 | m |
| 38.46 | 2.34 | m |
| 48.00 | 1.89 | s |

Example 5B

| | | |
|---|---|---|
| 9.82 | 9.00 | vs |
| 19.60 | 4.52 | w |
| 24.15 | 3.68 | m |
| 28.02 | 3.18 | m |
| 29.73 | 3.00 | w |
| 33.46 | 2.68 | w |
| 39.12 | 2.30 | w |
| 39.76 | 2.27 | w |
| 46.56 | 1.95 | w |
| 48.44 | 1.88 | w |
| 49.50 | 1.84 | w |

### Examples 6A & 6B

A Teflon beaker is charged with 75.00 g deionized water and placed under an overhead stirrer. With vigorous stirring, 15.85 g H₂O₂ (30 wt. %) and 17.89 g citric acid are added and dissolved. This is followed by the fast dropwise addition of 10.68 g Ti(OiPr)₄ (16.7% Ti), yielding a dark red-orange solution. Next, 3.76 g Fe(NO₃)₃*9H₂O solid is added slowly, forming a brownish-yellow solution. Then 16.96 g d-sorbitol is added, which dissolves while stirring. Separately, 30.41 g NaOH (98%) is dissolved in 50.85 g deionized water and allowed to stir and cool. The NaOH solution is added fast dropwise to the reaction mixture during which the color largely remains the same, and a dark brown-yellow solution results. The highly basic, clear solution is distributed among 6 Teflon-lined reaction vessels and digested quiescently at 125, 150, and 175° C. for 52 and 168 hr at autogenous pressures. Solid products are isolated by centrifugation, washed with deionized water, and dried at room temperature. PXRD is used to characterize the products. Characteristic diffraction lines for the Example 6A (150° C./52 hr) and Example 6B (175° C./168 hr) products are provided in Table 6. Elemental analysis via ICP yields the empirical composition with metals stoichiometry Na_{0.28}Fe_{0.18}Ti_{0.82} for the Example 6B material.

TABLE 6

| | | |
|---|---|---|
| 2-Φ | d(Å) | I/I₀% |

Example 6A

| | | |
|---|---|---|
| 8.12 | 10.88 | 100 |
| 24.49 | 3.63 | 3.6 |
| 28.26 | 3.16 | 23.2 |
| 33.61 | 2.66 | 6.7 |
| 38.34 | 2.35 | 4.1 |
| 47.94 | 1.90 | 31.3 |

Example 6B

| | | |
|---|---|---|
| 8.04 | 10.99 | w (sh) |
| 9.02 | 9.80 | vs |
| 9.65 | 9.16 | m (sh) |
| 24.20 | 3.68 | s |
| 27.94 | 3.19 | vs |
| 33.48 | 2.67 | s |
| 39.12 | 2.30 | s |
| 48.32 | 1.88 | m |
| 49.42 | 1.84 | w |

### Examples 7A & 7B

A Teflon beaker is charged with 75.00 g deionized water and placed under an overhead stirrer. With vigorous stirring, 13.47 g H₂O₂ (30 wt. %), 19.02 g citric acid, and 18.02 g d-sorbitol are added and dissolved. Next, 8.52 g Ti(OiPr)₄ (16.7% Ti) is added fast dropwise, resulting in a dark orange-red solution. Separately, 8.00 g Fe(NO₃)₃*9H₂O is dissolved in 15.00 g deionized water. This solution is added to the reaction mixture dropwise over a period of 3 minutes, and the reaction mixture remains a dark orange-red solution. Separately, 40.41 g NaOH (98%) is dissolved in 43.80 g deionized water and allowed to stir and cool. This solution is added to the reaction mixture fast dropwise, during which a dark brown color develops. Stirring post-addition, the reaction mixture becomes a clear dark red-brown solution. The highly basic, clear solution is distributed among 9 Teflon-lined reaction vessels and digested quiescently at 95, 125, 150, 175 and 190° C. for 48 and 169 hr at autogenous pressures. Solid products are isolated by centrifugation, washed with deionized water, and dried at room temperature. PXRD is used to characterize the products. Characteristic diffraction lines for the Example 7A (150° C./169 hr) and Example 7B (190° C./48 hr) products are provided in Table 7. Elemental analysis via ICP yields the empirical metals stoichiometries Na_{0.24}Fe_{0.37}Ti_{0.63} for the Example 7A material and Na_{0.33}Fe_{0.38}Ti_{0.62} for the Example 7B material.

TABLE 7

| | | |
|---|---|---|
| 2-Φ | d(Å) | I/I₀% |

Example 7A

| | | |
|---|---|---|
| 7.72 | 11.44 | vs |
| 15.35 | 5.77 | w |
| 24.75 | 3.59 | w |
| 28.20 | 3.17 | m |
| 33.70 | 2.66 | m |
| 35.38 | 2.54 | w |
| 38.18 | 2.36 | m |
| 39.10 | 2.30 | w |
| 43.48 | 2.080 | w |
| 48.04 | 1.89 | m |
| 50.03 | 1.82 | w |

Example 7B

| | | |
|---|---|---|
| 9.70 | 9.11 | vs |
| 24.10 | 3.69 | w |
| 27.90 | 3.20 | m |
| 29.91 | 2.99 | w |
| 33.40 | 2.68 | w |
| 35.34 | 2.54 | m |
| 39.22 | 2.30 | m |
| 42.84 | 2.11 | w |
| 46.59 | 1.95 | w |
| 48.40 | 1.88 | w |
| 49.34 | 1.84 | w |

### Example 8

This is a further example that illustrates alteration of the metal titanate structure via perturbation of what is a potassium iron titanate solution reaction mixture with LiCl, even though potassium is present in large excess. A Teflon beaker is charged with 90.00 g deionized water and placed under an overhead mixer. With vigorous stirring, 21.11 g H₂O₂ (30 wt. %), 17.89 g citric acid, and 16.96 g d-sorbitol are added and dissolved. Next, 3.76 g Fe(NO₃)₃*9H₂O solid is added slowly forming a yellow solution. This is followed by the fast dropwise addition of 10.68 g Ti(OiPr)₄ (16.7% Ti), yielding a dark red-orange solution. Separately, 59.99 g KOH (87.1%) is dissolved in 77.17 g deionized water and allowed to stir and cool. This solution is added to the reaction mixture fast dropwise, resulting in a dark brown-red solution. Separately, 3.96 g LiCl is dissolved in 12.00 g deionized water and added dropwise to the reaction mixture, which remains a red-brown solution. The highly basic clear solution is distributed among 8 Teflon-lined reaction vessels and digested quiescently at 125, 150, 175 and 190° C. for 53 and 169 hr at autogenous pressures. Solid products are isolated by centrifugation, washed with deionized water, and dried at room temperature. PXRD is used to characterize the products. Characteristic diffraction lines for the Example 8 (175° C./169 hr) product are provided in Table 8.

TABLE 8

| 2-Φ | d(Å) | I/I₀% |
|---|---|---|
| 10.58 | 8.36 | vs |
| 24.02 | 3.70 | w |
| 28.42 | 3.14 | s |
| 31.68 | 2.82 | w |
| 36.84 | 2.44 | m |
| 38.63 | 2.33 | w |
| 44.24 | 2.05 | m |
| 47.84 | 1.90 | w |
| 49.09 | 1.85 | w |
| 54.84 | 1.67 | w |

### Example 9

A Teflon beaker is charged with 90.00 g deionized water and placed under an overhead mixer. With vigorous stirring, 21.11 g H₂O₂ (30 wt. %), 17.89 g citric acid, and 16.96 g d-sorbitol are added and dissolved. Next, 3.00 g ZrOCl₂*8H₂O solid is added slowly, forming a clear, colorless solution. This is followed by the fast dropwise addition of 10.68 g Ti(OiPr)₄ (16.7% Ti), yielding a dark red-orange solution. Separately, 38.01 g NaOH (98%) is dissolved in 77.17 g deionized water and allowed to stir and cool. The NaOH solution is added to the reaction mixture fast dropwise, initiating a series of color changes that ultimately become a clear, colorless solution. The highly basic clear solution is distributed among 8 Teflon-lined reaction vessels and digested quiescently at 125, 150, 175 and 190° C. for 53 and 170 hr at autogenous pressures. Solid products are isolated by centrifugation, washed with deionized water, and dried at room temperature. PXRD is used to characterize the products. Characteristic diffraction lines for the Example 9 (150° C./170 hr) product are provided in Table 9. Elemental analysis via ICP yields the empirical metals composition Na_{0.39}Zr_{0.03}Ti_{0.97} for the Example 9 material.

TABLE 9

| 2-Φ | d(Å) | I/I₀% |
|---|---|---|
| 8.96 | 9.86 | vs |
| 24.43 | 3.64 | m |
| 28.18 | 3.16 | w |
| 48.14 | 1.89 | vs |

### Example 10

A Teflon beaker is charged with 75.00 g deionized water and placed under an overhead mixer. With vigorous stirring, 21.13 g H₂O₂ (30 wt. %), 17.89 g citric acid, and 16.96 g d-sorbitol are added and dissolved. Next, 4.24 g NH₄NbO(Ox)₂ (20.5% Nb, Ox=oxalate) solid is added slowly, forming a suspension. This is followed by the fast dropwise addition of 10.68 g Ti(OiPr)₄ (16.7% Ti), yielding a dark red-orange solution. Separately, 38.01 g NaOH (98%) is dissolved in 50.85 g deionized water and allowed to stir and cool. The NaOH solution is added to the reaction mixture fast dropwise, initiating a series of color changes that become a clear yellow solution. The reaction mixture is stirred for 90 minutes before it is distributed among 6 Teflon-lined reaction vessels and digested quiescently at 125, 150, and 175° C. for 65 and 169 hr at autogenous pressures. Solid products are isolated by centrifugation, washed with deionized water, and dried at room temperature. PXRD is used to characterize the products. Characteristic diffraction lines for the Example 10 (125° C./65 hr) product are provided in Table 10.

TABLE 10

| 2-Φ | d(Å) | I/I₀% |
|---|---|---|
| 9.60 | 9.21 | vs (br) |
| 24.25 | 3.67 | m (br) |
| 28.21 | 3.16 | s (br) |
| 48.02 | 1.89 | s (br) |

### Example 11

A Teflon beaker is charged with 75.00 g deionized water and placed under an overhead mixer. With vigorous stirring, 21.13 g H₂O₂ (30 wt. %) and 17.89 g citric acid are added and dissolved. Next, 4.24 g NH₄NbO(Ox)₂ (20.5% Nb, Ox=oxalate) solid is added slowly, forming a suspension. Then 16.96 g d-sorbitol is added to the suspension, followed by the fast dropwise addition of 10.68 g Ti(OiPr)₄ (16.7% Ti), yielding a dark red-orange solution/suspension. After stirring for an hour, the Ti/Nb reaction mixture is a red-orange solution. Separately, 59.98 g KOH (87.1%) is dissolved in 50.85 g deionized water and allowed to stir and cool. The KOH solution is added slow dropwise to the reaction mixture during which the color changes to light yellow. After the addition is completed, the reaction mixture is stirred for an additional 90 minutes during which it remains a clear yellow solution. The highly basic clear yellow solution is distributed among 6 Teflon-lined reaction vessels and digested quiescently at 125, 150, and 175° C. for 65 and 169 hr at autogenous pressures. Solid products are isolated by centrifugation, washed with deionized water, and dried at room temperature. PXRD is used to characterize the products. Characteristic diffraction lines for the Example 11 (150° C./169 hr) product are provided in Table 11.

TABLE 11

| 2-Φ | d(Å) | I/I₀% |
|---|---|---|
| 10.48 | 8.44 | s |
| 23.84 | 3.73 | w |
| 28.10 | 3.17 | vs |
| 47.60 | 1.91 | m |
| 48.88 | 1.86 | w |

### Example 12

A Teflon beaker is charged with 75.00 g deionized water and placed under an overhead mixer. With vigorous stirring, 15.85 g H₂O₂ (30 wt. %) and 17.89 g citric acid are added and dissolved. This is followed by the fast dropwise addition of 10.68 g Ti(OiPr)₄ (16.7% Ti), yielding a dark red-orange solution. Next, 3.76 g Fe(NO₃)₃*9H₂O solid is added slowly, forming a dark brown solution. Then 16.96 g d-sorbitol is added and dissolved and, with further stirring, the reaction mixture is a clear dark brown-yellow solution. Separately, 47.98 g KOH (87.1%) is dissolved in 50.85 g deionized water. This solution is added fast dropwise to the reaction mixture yielding a clear dark brown solution with a slight red tint. The highly basic clear brown solution is distributed among 6 Teflon-lined reaction vessels and digested quiescently at 125, 150, and 175° C. for 52 and 168 hr at autogenous pressures. Solid products are isolated by centrifugation, washed with deionized water, and dried at room temperature. PXRD is used to characterize the products. Characteristic diffraction lines for the Example 12 (175° C./168 hr) product are provided in Table 12. Elemental analysis via ICP yields the empirical metals composition K_{0.19}Fe_{0.29} Ti_{0.71} for the Example 12 material.

TABLE 12

| 2-Φ | d(Å) | I/I₀% |
|---|---|---|
| 9.70 | 9.11 | vs |
| 19.39 | 4.57 | w |
| 24.17 | 3.68 | w |
| 27.74 | 3.21 | m |
| 29.37 | 3.04 | w |
| 33.44 | 2.68 | w |
| 38.79 | 2.32 | w |
| 48.30 | 1.88 | w |
| 49.24 | 1.85 | w |

### Example 13

A Teflon beaker is charged with 100.00 g deionized water and placed under an overhead mixer. With vigorous stirring, 27.21 g H₂O₂ (30 wt. %), 15.37 g citric acid, and 14.57 g d-sorbitol are added and dissolved. This is followed by the fast dropwise addition of 18.34 g Ti(OiPr)₄ (16.7% Ti), yielding a dark red-orange solution. Next, 3.99 g solid Co(OAc)₂*4H₂O is added slowly to the reaction mixture, which becomes a red-purple solution. Separately, 39.18 g NaOH (98%) is dissolved in 44.16 g deionized water and allowed to stir and cool. This solution is added fast dropwise to the reaction mixture, yielding a dark black-green solution. The highly basic black-green solution is distributed among 9 Teflon-lined reaction vessels and digested quiescently at 95, 125, 150, 175 and 190° C. for 55 and 171 hr at autogenous pressures. Solid products are isolated by centrifugation, washed with deionized water, and dried at room temperature. PXRD is used to characterize the products. Characteristic diffraction lines for the Example 13 (150° C./171 hr) product are provided in Table 13. All products from the 150, 175 and 190° C. reactions exhibit this same XRD pattern. Elemental analysis via ICP on the 175° C./171 hr product yields the empirical metals composition Na_{0.26}Co_{0.20}Ti_{0.80}.

TABLE 13

| 2-Φ | d(Å) | I/I₀% |
|---|---|---|
| 7.72 | 11.44 | vs |
| 15.40 | 5.75 | w |
| 24.62 | 3.61 | w |
| 28.04 | 3.18 | m |
| 33.66 | 2.66 | m |
| 38.16 | 2.36 | m |
| 43.60 | 2.07 | w |
| 47.80 | 1.90 | m |
| 50.00 | 1.82 | w |

### Examples 14A & 14B

A 1 L Teflon beaker is charged with 320.00 g deionized water and placed under an overhead mixer in an ice bath. With vigorous stirring, 95.23 g H₂O₂ (30 wt. %), 53.80 g citric acid, and 51.01 g d-sorbitol are added and dissolved. This is followed by the fast dropwise addition of 64.20 g Ti(OiPr)₄ (16.7% Ti), yielding some precipitation and a dark red-orange solution. The solids dissolve with further stirring. Next, 13.95 g Co(OAc)₂*4H₂O is slowly added to the reaction mixture and dissolved after 10 minutes of stirring. Separately, 137.14 g NaOH (98%) is dissolved in 184.56 g deionized water and placed in an ice bath. The cooled NaOH solution is added fast dropwise to the reaction mixture, forming a dark blue-green solution. The reaction mixture is stirred further, the clear solution becoming green with some blue tint by the time it reaches room temperature. The highly basic green-blue solution is transferred to a 1 L Teflon-lined Parr reactor and digested quiescently at 150° C. for 168 hr at autogenous pressure. Solid product is isolated by centrifugation, washed with deionized water, and dried at room temperature. PXRD is used to characterize the product. Characteristic diffraction lines for the Example 14A product are provided in Table 14. Elemental analysis via ICP yields the empirical metals composition Na_{0.40}Co_{0.19}Ti_{0.81}.

A 2.0 g portion of the product was ion-exchanged using 0.5 M Mg(NO₃)₂ solution (100 mL) at room temperature. The exchange is carried out 3 times before washing the product with deionized water and drying in air. The Example 14B product is characterized by PXRD, and characteristic diffraction lines are provided in Table 14. The Mg²⁺ exchanged product exhibits a different XRD pattern and has higher crystallinity than the parent.

TABLE 14

| | | |
|---|---|---|
| 2-Theta | d(Å) | A % |

Example 14A

| | | |
|---|---|---|
| 10.14 | 8.72 | vs |
| 20.33 | 4.36 | w |
| 24.00 | 3.71 | m |
| 28.12 | 3.17 | s |
| 30.92 | 2.89 | w |
| 33.66 | 2.66 | m |
| 38.32 | 2.35 | w |
| 39.66 | 2.27 | m |
| 47.90 | 1.90 | m |
| 49.14 | 1.85 | w |
| 49.86 | 1.83 | w |

Example 14B

| | | |
|---|---|---|
| 7.84 | 11.26 | vs |
| 15.82 | 5.60 | m |
| 23.80 | 3.74 | w |
| 26.28 | 3.39 | m |
| 30.80 | 2.90 | w |
| 31.94 | 2.80 | w |
| 35.84 | 2.50 | w |
| 41.08 | 2.20 | w |
| 47.96 | 1.90 | w |
| 50.62 | 1.80 | w |

### Examples 15A & 15B

A Teflon beaker is charged with 100.00 g deionized water and placed under an overhead mixer. With vigorous stirring, 27.21 g H₂O₂ (30 wt. %), 15.37 g citric acid, and 14.57 g d-sorbitol are added and dissolved. This is followed by the fast dropwise addition of 18.34 g Ti(OiPr)₄ (16.7% Ti), yielding a dark red-orange solution. Next, 3.92 g solid Mn(OAc) 2*4H₂O is added slowly to the reaction mixture, which becomes a red-purple solution. Separately, 39.18 g NaOH (98%) is dissolved in 44.16 g deionized water and allowed to stir and cool. This solution was added fast dropwise to the reaction mixture, and initially yields a dark orange-brown solution that darkens to brown. The highly basic dark brown solution is distributed among 9 Teflon-lined reaction vessels and digested quiescently at 95, 125, 150, 175 and 190° C. for 54 and 170 hr at autogenous pressures. Solid products are isolated by centrifugation, washed with deionized water, and dried at room temperature. PXRD is used to characterize the products. Characteristic diffraction lines for the Example 15A (125° C./170 hr) and Example 15B (150° C./170 hr) products are provided in Table 15. Elemental analysis via ICP yields the empirical composition Na_{0.25}Mn_{0.20}Ti_{0.80} for the Example 15B material.

TABLE 15

| | | |
|---|---|---|
| 2-Theta | d(Å) | I/I₀% |

Example 15A

| | | |
|---|---|---|
| 8.74 | 10.11 | vs |
| 9.08 | 9.73 | vs |
| 24.43 | 3.64 | w |
| 27.94 | 3.19 | m |
| 27.94 | 3.19 | m |
| 33.62 | 2.66 | w |
| 38.68 | 2.33 | w |
| 47.80 | 1.90 | m |

Example 15B

| | | |
|---|---|---|
| 7.70 | 11.47 | vs |
| 8.51 | 10.38 | m |
| 8.90 | 9.93 | w |
| 15.33 | 5.78 | w |
| 24.52 | 3.63 | w |
| 28.06 | 3.18 | m |
| 33.56 | 2.67 | w |
| 38.10 | 2.36 | w |
| 38.56 | 2.33 | w |
| 43.52 | 2.08 | w |
| 47.64 | 1.91 | w |
| 49.96 | 1.82 | w |

### Examples 16-26: Synthesis of Metal Titanates from TiO₂ Powders and Formed TiO₂ Spheres

Performance of hydrogen peroxide and complexing agents, including at least one MHCA, in dissolving and stabilizing Ti-M-containing species in highly alkaline reaction mixtures, as well as facilitating the formation of the metal titanate ion exchange compositions, are investigated by applying MHCAs in alkali hydroxide solutions to transform various solid TiO₂ sources, including TiO₂ powders and formed spray dried TiO₂ spheres to alkali titanate and alkali metal titanate ion exchangers. In the aspect of pure alkali titanates, only alkali hydroxide solutions containing the MHCA are required to carry out the transformation. In the aspect of incorporation of the M metals into the solid TiO₂, the M metal is dissolved in the alkali hydroxide solution using a combination of complexing agents as seen in Examples 1-15, such as hydrogen peroxide, citric acid and a MHCA such as d-sorbitol; the citric acid required to stabilize the M metal in moderately basic solution, while d-sorbitol keeps the M metal in solution as the reaction mixture is adjusted to the very high pH required to hydrothermally synthesize the metal titanate ion exchange composition. Similar to the homogenous solution approach disclosed in Examples 1-15, metal incorporation is seen, and the product consists of large polycrystalline particulates of desirable sizes, such that absorption in the gastrointestinal tract would be avoided. Examples 26 and 27 describe product particulate size.

### Synthesis of Spray Dried TiO₂ Spheres

Spray dried TiO₂ spheres are synthesized with the Yamato Spray Drier Model DL-41. Typically, 500 grams of a slurry with 20 wt. % titanium dioxide powder in DI H₂O is prepared. To 400 g of deionized water, 100 g of fumed TiO₂ powder (Degussa D-6000) is added while mixing with an overhead mixer at 500 RPM. The mixture is stirred for 10 minutes. The suspension is then Eiger milled for 15 minutes before spray drying. Larger agglomerates are removed by passing the suspension through a 100-mesh (150 μm) sieve. The suspension is stirred continuously to avoid settling of particles. The spray drier chamber temperature reached 110° C. with a drying air flow of 80 SCFH. The aspirator flow is 0.8 cm3/min with 10 psi head pressure. The slurry feeding speed used for this process is 16 cc/min. The collected product is screened through 60-mesh (250 μm), 100-mesh (150 μm), and 200-mesh (75 μm) sieves; the final sample collected has particles finer than 200-mesh (75 μm). Spray dried TiO₂ spheres from a typical preparation are characterized in Example C5.

### Example 16

This preparation starts with a freshly precipitated titania source. In a 1 L flask equipped with an overhead stirrer, 192.5 g NH₄OH (28% NH₃) is diluted in 365.25 g deionized water with stirring. A dry pressure-equalizing dropping funnel is added and charged with 50.00 g TiCl₄. The TiCl₄ is added dropwise and intermittently to avoid excessive heating of the reaction mixture. White solid forms in the flask, filling the flask by the end of the addition. The reaction mixture is stirred and cooled for an additional 45 minutes post addition before distributing the mixture into centrifuge bottles for isolation and washing with deionized water. The wet cake is stored in a sealed vessel, and it contains 11.2% Ti. This is used as the starting material for the next step.

To a small beaker placed under an overhead stirrer, 10.74 g NaOH (98%) is dissolved in 34.77 g deionized water with stirring and allowed to cool. Then 1.45 g catechol is added, forming a green-yellow solution as it dissolves. Freshly precipitated titania (11.2% Ti) from the preparation above is added, forming a greenish yellow suspension. After further stirring, the highly basic reaction mixture is distributed among 3 Teflon-lined reaction vessels and digested quiescently at 95 and 150° C. for 96 hr, and at 190° C. for 49 hr, at autogenous pressures. Solid products are isolated by centrifugation, washed with deionized water, and dried at room temperature. PXRD is used to characterize the products. Characteristic diffraction lines for the Example 16 (190° C./49 hr) product are provided in Table 16. Elemental analysis via ICP yields the empirical metals composition Na_{0.37}Ti_{1.00} for the Example 16 material.

TABLE 16

| 2-Φ | d(Å) | I/I₀% |
|---|---|---|
| 9.21 | 9.60 | w (sh) |
| 9.82 | 9.00 | vs |
| 15.86 | 5.58 | w |
| 19.71 | 4.50 | w |
| 24.96 | 3.56 | w |
| 28.98 | 3.08 | w |
| 29.46 | 3.03 | m |
| 34.48 | 2.60 | m |
| 35.08 | 2.56 | m |
| 38.82 | 2.32 | w |
| 42.80 | 2.11 | w |
| 45.49 | 1.99 | w |
| 48.38 | 1.88 | w |
| 49.50 | 1.84 | w |
| 52.66 | 1.74 | w |

### Example 17

A Teflon beaker is charged with 12.88 g of 8.5 M KOH solution. With magnetic bar stirring, 0.69 g catechol reagent is added and dissolved, creating a clear, light-brown solution. After 5 minutes of mixing, 1 g of TiO₂ powder is added, rendering the mixture opaque. Reaction contents are mixed for 30 minutes, resulting in a cream-brown opaque mixture. The highly basic solution is loaded into a Teflon-lined 45 cc reaction vessel and digested at 200° C. for 20 hours with tumbling (40 rpm) at autogenous pressure. The solid product is isolated via filtration, washed with copious amount of deionized water, and dried at 100° C. PXRD is used to characterize the product. Characteristic diffraction lines for the product are provided in Table 17. Elemental analysis via ICP yields the metals stoichiometry K_{0.26}Ti_{1.00}.

TABLE 17

| 2-Φ | d(Å) | I/I₀% |
|---|---|---|
| 10.86 | 8.14 | m |
| 24.11 | 3.69 | m |
| 29.15 | 3.06 | vs |
| 33.66 | 2.66 | m |
| 42.94 | 2.10 | m |
| 47.80 | 1.90 | m |
| 59.15 | 1.56 | w |
| 65.66 | 1.42 | w |

### Example 18

A Teflon beaker is charged with 5.00 g deionized water and placed under an overhead mixer. With vigorous stirring, 0.48 g citric acid, 1.60 g d-sorbitol, and 1.01 g Fe(NO₃)₃*9H₂O are added and dissolved, creating a clear purple solution. Next, with vigorous stirring, 14.12 g of a cooled 8.5 M KOH solution is added dropwise over 7 minutes, resulting in a clear dark green solution. Finally, to the resulting dark green solution, 1.00 g TiO₂ powder is added and allowed to homogenize over a 30-minute period. The resulting dark green, highly basic suspension is distributed into Teflon-lined 45 ml reaction vessels and digested quiescently at 200° C. for 4 days at autogenous pressures. Solid product is isolated by centrifugation, washed with deionized water, and dried at 100° C. PXRD is used to characterize the product. Characteristic diffraction lines for the Example 18 product are provided in Table 18. Elemental analysis via ICP yields the metals stoichiometry K_{0.31}Fe_{0.18}Ti_{0.82}.

TABLE 18

| 2-Φ | d(Å) | I/I₀% |
|---|---|---|
| 9.86 | 8.96 | vs |
| 24.12 | 3.69 | w |
| 27.95 | 3.19 | m |
| 33.46 | 2.68 | w |
| 39.01 | 2.31 | w |
| 48.00 | 1.89 | w |
| 49.29 | 1.85 | w |

### Example 19

A Teflon beaker is charged with 5.00 g deionized water and placed under an overhead mixer. With vigorous stirring, 0.48 g citric acid, 1.60 g d-sorbitol, and 1.01 g Fe(NO₃)₃*9H₂O are added and dissolved, resulting in a clear purple solution. Next, with vigorous stirring, 15.02 g of a cooled 7.5 M NaOH solution is added dropwise over 7 minutes, resulting in a green solution. Finally, to the resulting light green solution, 1.00 g TiO₂ powder is added and allowed to homogenize over a 30-minute period. The resulting green opaque, highly basic suspension is distributed to Teflon-lined reaction vessels and digested quiescently at 200° C. for 1 and 4 days at autogenous pressures. Solid products are isolated by centrifugation, washed with deionized water, and dried at 100° C. PXRD is used to characterize the products. Characteristic diffraction lines for the Example 19A (200° C./1d) and Example 19B (200° C./4d) products are provided in Table 19. Elemental analyses via ICP yields the metals stoichiometry Na_{0.33}Fe_{0.18}Ti_{0.82} for the Example 19A product and Na_{0.46}Fe_{0.17}Ti_{0.83} for the Example 19B product.

TABLE 19

| | | |
|---|---|---|
| 2-Φ | d(Å) | I/I₀% |

Example 19A

| | | |
|---|---|---|
| 9.74 | 9.07 | vs |
| 24.27 | 3.67 | w |
| 25.28 | 3.52 | w |
| 27.85 | 3.20 | w |
| 28.23 | 3.16 | w |
| 28.79 | 3.10 | w |
| 33.60 | 2.67 | w |
| 39.26 | 2.29 | w |
| 48.38 | 1.88 | w |
| 49.37 | 1.84 | w |

Example 19B

| | | |
|---|---|---|
| 7.95 | 11.12 | vs |
| 9.24 | 9.56 | w |
| 24.56 | 3.62 | w |
| 28.17 | 3.16 | m |
| 33.58 | 2.67 | w |
| 38.31 | 2.35 | w |
| 43.79 | 2.07 | w |
| 48.04 | 1.89 | w |
| 56.09 | 1.64 | w |
| 62.63 | 1.48 | w |

### Example 20

A Teflon beaker is charged with 5.00 g deionized water and a magnetic stir bar is added. With vigorous stirring, 1.14 g d-sorbitol is added and dissolved to form a clear solution. Next, with continued stirring, 15.71 g of a cooled 7.5 M NaOH solution is added dropwise over 7 minutes. Finally, to the resulting highly basic solution, 1.00 g TiO₂ spray dried spheres are added and allowed to gently homogenize over 2 minutes. The resulting suspension is transferred to a Teflon-lined 45 ml reaction vessel and digested quiescently at 200° C. for 4 days at autogenous pressure. Solid product is isolated by filtration, washed with deionized water, and dried at 100° C. PXRD is used to characterize the Example 20A product, and characteristic diffraction lines are provided in Table 20 A portion of the resulting novel Example 20A sphere product is acid treated at room temperature using a diluted nitric acid solution to adjust the slurry to a steady PH ˜2. This product is Example 20B.

TABLE 20

| 2-Θ | d(Å) | I/I₀ % |
|---|---|---|
| 7.89 | 11.20 | vs |
| 8.53 | 10.36 | w |
| 11.78 | 7.51 | w |
| 14.27 | 6.20 | w |
| 15.73 | 5.63 | w |
| 16.72 | 5.30 | w |
| 17.11 | 5.18 | w |
| 17.72 | 5.00 | w |
| 18.04 | 4.91 | w |
| 19.04 | 4.66 | w |
| 19.81 | 4.48 | w |
| 20.95 | 4.24 | w |
| 22.28 | 3.99 | w |
| 22.39 | 3.97 | w |
| 26.38 | 3.38 | w |
| 27.76 | 3.21 | w |
| 28.20 | 3.16 | w |
| 28.61 | 3.12 | w |
| 30.54 | 2.92 | w |
| 31.68 | 2.82 | w |
| 32.01 | 2.79 | w |
| 32.27 | 2.77 | w |
| 32.74 | 2.73 | w |
| 33.70 | 2.66 | w |
| 34.17 | 2.62 | w |
| 35.08 | 2.56 | w |
| 35.81 | 2.51 | w |
| 36.69 | 2.45 | w |
| 37.17 | 2.43 | w |
| 37.89 | 2.37 | w |
| 38.25 | 2.35 | w |
| 38.49 | 2.34 | w |
| 39.40 | 2.29 | w |
| 39.88 | 2.26 | w |
| 40.14 | 2.24 | w |
| 40.66 | 2.22 | w |
| 41.17 | 2.19 | w |
| 41.68 | 2.17 | w |
| 42.40 | 2.13 | w |
| 42.85 | 2.11 | w |
| 43.74 | 2.07 | w |
| 45.18 | 2.01 | w |
| 45.63 | 1.99 | w |
| 46.29 | 1.96 | w |
| 46.70 | 1.94 | w |
| 53.18 | 1.72 | w |
| 53.39 | 1.71 | w |
| 54.11 | 1.69 | w |
| 54.94 | 1.67 | w |
| 57.60 | 1.60 | w |
| 58.28 | 1.58 | w |
| 58.99 | 1.56 | w |
| 59.37 | 1.56 | w |
| 59.89 | 1.54 | w |

### Example 21

A Teflon beaker is charged with 5.00 g deionized water and a magnetic stir bar. With vigorous stirring, 0.48 g citric acid, 1.60 g d-sorbitol are dissolved, forming a clear solution. Next, with continued stirring, 1.01 g Fe(NO₃)₃*9H₂O is added, yielding a clear lavender colored solution. To this reaction mixture, 15.02 g of a cooled 7.5 M NaOH solution is added dropwise over 7 minutes. Finally, to the resulting highly basic green solution, 1.00 g TiO₂ spray dried spheres are added, and the mixture is allowed to gently homogenize over 2 minutes. The resulting neon lime-green slurry is loaded into a Teflon-lined 45 ml reaction vessel and digested quiescently at 200° C. for 4 days at autogenous pressure. Solid product is isolated by filtration, washed with deionized water, and dried at 100° C. PXRD is used to characterize the Example 21A product, and characteristic lines are provided in Table 21. A portion of the Example 21A sphere product is acid treated at room temperature using diluted nitric acid to adjust the slurry to a steady pH ˜2. This product is Example 21B.

TABLE 21

| 2-Θ | d(Å) | I/I₀ % |
|---|---|---|
| 9.02 | 9.79 | s |
| 9.90 | 8.93 | vs |
| 24.29 | 3.66 | m |
| 25.38 | 3.51 | m |
| 27.49 | 3.24 | w |
| 28.10 | 3.17 | m |
| 28.49 | 3.13 | m |
| 37.99 | 2.37 | w |
| 48.06 | 1.89 | m |
| 48.42 | 1.88 | m |

### Example 22

A 100 ml polypropylene beaker is charged with 31.42 g of 7.5 M NaOH solution and placed under an overhead mixer. With vigorous mixing, 2.28 g of d-sorbitol is added and mixed until dissolved. Then 2 g of TiO₂ spray dried spheres are added. After allowing the reaction mixture to stir for 5 minutes, the reaction mixture is transferred to two 45 ml Teflon-lined Parr vessels and digested with tumbling (40 rpm) at 175° C. for 24 and 96 hr at autogenous pressure. Solid products are isolated by centrifugation, washed with deionized water, and dried at 100° C. overnight. PXRD is used to characterize the products. Characteristic diffraction lines for Example 22 (175° C./96 hr) product are provided in Table 22.

TABLE 22

| 2-Θ | d(Å) | I/I₀ % |
|---|---|---|
| 7.77 | 11.38 | vs |
| 14.17 | 6.25 | w |
| 15.61 | 5.67 | w |
| 16.63 | 5.33 | w |
| 17.02 | 5.20 | w |
| 17.62 | 5.03 | w |
| 17.96 | 4.94 | w |
| 18.94 | 4.68 | w |
| 19.72 | 4.50 | w |
| 20.85 | 4.26 | w |
| 22.27 | 3.99 | w |
| 26.32 | 3.38 | w |
| 27.62 | 3.23 | w |
| 28.12 | 3.17 | w |
| 28.55 | 3.12 | w |
| 30.45 | 2.93 | w |
| 31.54 | 2.83 | w |
| 31.91 | 2.80 | w |
| 32.20 | 2.78 | w |
| 33.58 | 2.67 | w |
| 35.00 | 2.56 | w |
| 35.73 | 2.51 | w |
| 36.59 | 2.45 | w |
| 37.07 | 2.42 | w |
| 37.79 | 2.38 | w |
| 38.21 | 2.35 | w |
| 39.77 | 2.27 | w |
| 40.06 | 2.25 | w |
| 40.56 | 2.22 | w |
| 41.06 | 2.20 | w |
| 41.56 | 2.17 | w |
| 42.32 | 2.13 | w |
| 42.76 | 2.11 | w |
| 43.65 | 2.07 | w |
| 45.09 | 2.01 | w |
| 45.56 | 1.99 | w |
| 46.23 | 1.96 | w |
| 46.62 | 1.95 | w |
| 57.40 | 1.60 | w |
| 58.22 | 1.58 | w |
| 58.93 | 1.57 | w |
| 59.28 | 1.56 | w |
| 59.83 | 1.54 | w |
| 61.30 | 1.51 | w |
| 66.90 | 1.40 | w |

### Example 23

A 100 ml polypropylene beaker is charged with 25.75 g of 8.5 M KOH solution and placed under an overhead mixer. With vigorous mixing, 2.28 g of d-sorbitol is added and mixed until dissolved. Then 2 g of TiO₂ spray dried spheres are added. After allowing the reaction mixture to stir for 5 minutes, the reaction mixture is transferred to two 45 ml Teflon-lined Parr vessels and digested with tumbling (40 rpm) at 200° C. for 24 and 96 hr at autogenous pressure. Solid products are isolated by centrifugation, washed with deionized water, and dried at 100° C. overnight. PXRD is used to characterize the products. Characteristic diffractions lines for the Example 23A (200° C./96 hr) product are provided in Table 23. A 1.0 g portion of the Example 23A product is acid treated using 10 g deionized water with the pH being adjusted to 3 using 1 M HNO₃ solution. Once the powder is added to pH 3 solution, the mixture's pH is adjusted to pH 1.5-2.0 by addition of 1 M HNO₃ solution with stirring. The mixture is stirred at room temperature for 30 minutes before washing the product with deionized water and drying at 80° C. overnight. This Example 23B product is characterized by PXRD, and characteristic diffraction lines are provided in Table 23.

TABLE 23

| Example 23A | | | Example 23B | | |
|---|---|---|---|---|---|
| 2-Θ | d(Å) | I/I₀ % | 2-Θ | d(Å) | I/I₀ % |
| 11.14 | 7.94 | w | 11.25 | 7.86 | m |
| 23.98 | 3.71 | w | 24.23 | 3.67 | vs |
| 29.21 | 3.05 | vs | 29.65 | 3.01 | vs |
| 31.35 | 2.85 | s | 33.73 | 2.65 | m |
| 33.60 | 2.67 | s | 42.98 | 2.10 | m |
| 34.40 | 2.60 | s | 48.06 | 1.89 | vs |
| 42.61 | 2.12 | m | 59.89 | 1.54 | w |
| 47.74 | 1.90 | m | 66.02 | 1.41 | w |
| 59.28 | 1.56 | w | | | |
| 65.73 | 1.42 | w | | | |

### Example 24

A 2-liter polypropylene beaker is charged with 901.6 g of 8.5 M KOH solution and placed under an overhead mixer. With vigorous mixing at 300 rpm, 79.8 g of d-sorbitol is added and mixed until dissolved. Then added 70 g TiO₂ powder (Degussa) is added, and the reaction mixture is stirred for 15 minutes. The reaction mixture is placed in a 2 L Parr stirred reactor and digested at 200° C. for 18 hr at autogenous pressure while stirring at 40 rpm. The solid product is isolated by centrifugation, washed with deionized water, and dried at 80° C. overnight. PXRD is used to characterize the product. Characteristic diffraction lines for the Example 24A product are provided in Table 24. Elemental analyses via ICP yields the metals stoichiometry K_{0.40}Ti_{1.00} for the Example 24A product. This preparation is repeated 10 times and the products are combined to make a composite sample, Example 24E, which exhibits metals stoichiometry K_{0.30}Ti. Characteristic PXRD diffraction lines for the Example 24E composite product are provided in Table 24.

A 5.0 g portion of the Example 24A product is acid treated using 50 g deionized water adjusted to pH=3 using 1 M HNO₃ solution. Once addition of the Example 24A powder to the stirred pH=3 solution is complete, the pH is further adjusted to 1.5-2.0 by addition of 1M HNO₃ solution. The mixture stirred at room temperature for 30 minutes before washing the product with deionized water and drying at 80° C. overnight. The Example 24B product is characterized by PXRD, and characteristic diffraction lines are provided in Table 24. A portion of the Example 24E composite product is also acid treated in the same manner to obtain the Example 24F product. Elemental analysis via ICP yields the metals stoichiometry K_{0.15}Ti for the Example 24F product. The acid treated Example 24F product is also characterized by PXRD, and characteristic diffraction lines are provided in Table 24.

A 4.0 g portion of the Example 24A product is calcined at 350° C. for 2 h under flowing clean dry air at a flow rate of 300 SCFH. The Example 24C product is characterized by PXRD, and characteristic diffraction lines are provided in Table 24. The calcined product exhibits a similar XRD pattern with similar crystallinity to the Example 24A parent sample.

A 2.0 g portion of the calcined Example 24C product is acid treated using 20 g deionized water adjusted to pH=3 by addition of 1 M HNO₃ solution with stirring. Once the Example 24C powder is added to the pH=3 solution, the pH of the mixture is adjusted to 1.5-2.0 by addition of 1 M HNO₃ solution with continued stirring. The mixture is stirred at room temperature for an additional 30 minutes before washing the product with deionized water and drying at 80° C. overnight. The Example 24D product is characterized by PXRD, and characteristic diffraction lines are provided in Table 24.

TABLE 24

| Example 24A | | | Example 24B | | | Example 24C | | |
|---|---|---|---|---|---|---|---|---|
| 2-Θ | d(Å) | I/I₀% | 2-Θ | d(Å) | I/I₀% | 2-Θ | d(Å) | I/I₀% |
| 10.86 | 8.14 | w (br) | 11.08 | 7.98 | w (br) | 11.26 | 7.85 | m |
| 11.16 | 7.92 | w (br) | 24.17 | 3.68 | s (br) | 24.11 | 3.69 | m |
| 24.15 | 3.68 | w (br) | 24.52 | 3.63 | m (sh) | 29.39 | 3.04 | vs |
| 29.05 | 3.07 | vs (br) | 29.34 | 3.04 | s (vb) | 33.64 | 2.66 | m |
| 33.46 | 2.68 | m (br) | 33.59 | 2.67 | m (vb) | 42.73 | 2.11 | m |
| 42.96 | 2.10 | m (br) | 42.48 | 2.13 | m (br) | 47.80 | 1.90 | m |
| 47.84 | 1.90 | m | 42.91 | 2.11 | m (br) | 59.35 | 1.56 | w |
| 59.57 | 1.55 | w (br) | 48.08 | 1.89 | vs | 66.13 | 1.41 | w |
| 66.05 | 1.41 | w (br) | 48.56 | 1.87 | w (sh) | | | |
| | | | 59.37 | 1.56 | w (br) | | | |
| | | | 66.32 | 1.41 | w (br) | | | |

| Example 24D | | | Example 24E | | | Example 24F | | |
|---|---|---|---|---|---|---|---|---|
| 2-Θ | d(Å) | I/I₀% | 2-Θ | d(Å) | I/I₀% | 2-Θ | d(Å) | I/I₀% |
| 11.22 | 7.89 | m | 11.02 | 8.02 | w (br) | 11.34 | 7.80 | w (br) |
| 24.10 | 3.69 | m | 24.12 | 3.69 | m | 24.19 | 3.68 | s |
| 29.10 | 3.07 | vs | 29.13 | 3.06 | vs (br) | 29.11 | 3.06 | vs (br) |
| 33.46 | 2.68 | m | 31.36 | 2.85 | m (sh) | 31.25 | 2.86 | w (sh) |
| 42.87 | 2.11 | m | 33.50 | 2.67 | m (br) | 33.34 | 2.69 | m (br) |
| 47.88 | 1.90 | m | 42.82 | 2.11 | m (br) | 42.54 | 2.12 | m (br) |
| 66.02 | 1.41 | m | 47.76 | 1.90 | m | 43.11 | 2.10 | m (br) |
| | | | 59.95 | 1.54 | m (br) | 48.00 | 1.89 | vs |
| | | | 66.28 | 1.41 | m (br) | 59.40 | 1.55 | w (br) |
| | | | | | | 66.05 | 1.41 | w (br) |

### Example 25

A standard 8.5 M KOH solution is prepared by dissolving 560 g KOH (85%) in 500 g deionized water, and allowing the resulting solution to stir and cool. The solution is transferred to a one-liter volumetric flask and diluted to 1000 ml. This standard KOH solution is used in the following preparations, and the process is repeated as more solution is required.

A 2 L static Parr reactor equipped with a Teflon liner is employed for the following synthesis, which is carried out three times. The Teflon liner is charged with 811.40 g of the standard KOH solution. To this solution, 71.96 g sorbitol is added, and the mixture is stirred until all solids dissolve. Once a clear solution is achieved, the reaction mixture is charged with 63.30 g preformed spray dried TiO₂ spheres. The mixture is stirred gently with a stirbar for 15 minutes. The stirbar is then removed from the reaction mixture, and the Teflon liner with reaction mixture is placed in the 2 L Parr reactor. The reactor is sealed and the reaction mixture is digested quiescently at 200° C. for 4 days at autogenous pressure. After digestion, the reaction mixture is cooled, the mother liquor decanted, and the solid product isolated via filtration. The solid product is washed with 6 L deionized water and dried at 100° C. This reaction is carried out three times, Examples 25A, 25B and 25C. These products are characterized by PXRD and are found to be nearly identical. Characteristic diffraction lines are provided in Table 25. The Example 25A, 25B and 25C samples are combined to form a composite product, Example 25D. Elemental analysis via ICP yields the metals stoichiometry K_{0.28}Ti for the Example 25D composite product. A portion of this composite product is acid treated, forming the Example 25E product. To 2100 g deionized water in a 4 L beaker, 205.74 g of the Example 25D composite product is suspended with stirring. The pH is adjusted to 3 via the dropwise addition of concentrated nitric acid. The pH is further adjusted until it was stable at pH=2 via the addition of 1M HNO₃. The resulting slurry is stirred about an hour at room temperature with periodic adjustment of the pH to keep it at 2. The solid Example 25E product is isolated by filtration, washed with 4 L of deionized water, and dried at 100° C. The Example 25D and 25E products are characterized by PXRD, and representative diffraction lines are provided in Table 25. Elemental analyses by ICP yields a metals stoichiometry of K_{0.28}Ti for the parent Example 25D composite product and K_{0.16}Ti for the acid-treated Example 25E product.

TABLE 25

| Example 25A | | | Example 25B | | | Example 25C | | |
|---|---|---|---|---|---|---|---|---|
| 2-Θ | d(Å) | I/I₀% | 2-Θ | d(Å) | I/I₀% | 2-Theta | d(Å) | I/I₀% |
| 11.42 | 7.74 | m (br) | 11.11 | 7.95 | s (br) | 11.43 | 7.73 | m (br) |
| 24.09 | 3.69 | m | 23.92 | 3.72 | w | 24.22 | 3.67 | w |
| 29.49 | 3.03 | vs (br) | 29.19 | 3.06 | vs (br) | 29.29 | 3.05 | vs (br) |
| 31.66 | 2.82 | m (sh) | 31.11 | 2.87 | s (sh) | 31.26 | 2.86 | w (sh) |
| 33.66 | 2.66 | m (br) | 33.44 | 2.68 | m (br) | 33.72 | 2.66 | m (br) |
| 42.92 | 2.11 | m (br) | 42.76 | 2.11 | m (br) | 42.99 | 2.10 | m (br) |
| 47.88 | 1.90 | m | 47.74 | 1.90 | m | 47.76 | 1.90 | m |
| 59.77 | 1.55 | w (br) | 59.20 | 1.56 | m (br) | 59.44 | 1.55 | w (br) |
| 66.07 | 1.41 | w (br) | 66.02 | 1.41 | m (br) | 66.16 | 1.41 | w (br) |

| | |
|---|---|
| Example 25D | Example 25E |

| 2-Θ | d(Å) | I/I₀% | 2-Θ | d(Å) | I/I₀% |
|---|---|---|---|---|---|
| 11.62 | 7.61 | m (br) | 11.26 | 7.85 | s (br) |
| 24.30 | 3.66 | m | 24.23 | 3.67 | m |
| 29.25 | 3.05 | vs (br) | 29.31 | 3.04 | vs (br) |
| 31.39 | 2.85 | m (sh) | 30.99 | 2.88 | w (sh) |
| 33.60 | 2.67 | m (br) | 33.65 | 2.66 | m (br) |
| 42.77 | 2.11 | m (br) | 43.05 | 2.10 | vs (br) |
| 47.82 | 1.90 | m | 48.06 | 1.89 | s |
| 59.64 | 1.55 | w (br) | 59.62 | 1.55 | w (br) |
| 65.99 | 1.41 | w (br) | 66.20 | 1.41 | w (br) |

### Example 26

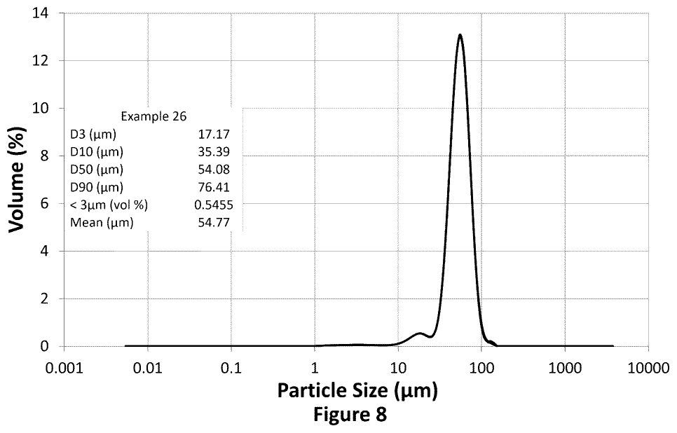

This example uses nano-sized titania powder as the Ti source. A 100 ml polypropylene beaker was charged with 40 g of pre-made 8.5 M KOH solution and placed under an overhead mixer. D-sorbitol, 4.56 g, was added to the KOH solution and stirred until dissolved. With vigorous mixing, 4 g of pure anatase nano-sized TiO₂ powder (Kemira), was added, allowing the reaction mixture to stir for 30 minutes. A portion of the reaction mixture was placed in a 45 ml Teflon-lined Parr vessel and digested quiescently at 200° C. for 24 hrs at autogenous pressure. The solid product was isolated by centrifugation, washed with deionized water, and dried at 100° C. overnight. Particle size distribution for the Example 26 product is shown in FIG. 8 and discussed in Example 27.

### Comparative Examples C1-C6

The following Comparative Examples are described.

### Example C1

A sample of sodium nonatitanate from Allied-Signal is disclosed for Pb uptake from bodily fluids in U.S. Pat. No. 11,964,266, Example 4. Characterization of the sample by PXRD is consistent with sodium nonatitanate. Representative x-ray diffraction lines for the sample are provided in Table 26. Elemental analysis by ICP yields the metals stoichiometry Na_{0.5}Ti.

TABLE 26

| 2-Θ | d(Å) | I/I₀ % |
|---|---|---|
| 9.22 | 9.58 | vs |
| 10.04 | 8.80 | w |
| 24.03 | 3.701 | w |
| 28.07 | 3.18 | w (br) |
| 33.66 | 2.66 | w (br) |
| 39.57 | 2.28 | w (br) |
| 48.08 | 1.89 | w (br) |

### Example C2

A commercial potassium octatitanate, product number HON393 from Honeywell Specialty Chemicals, Seelze GMBH is disclosed for Pb²⁺ uptake from bodily fluids in U.S. Pat. No. 11,964,266, Example 17. This sample is a composite consisting of potassium octatitanate, K₂Ti₈O₁₇, but also containing anatase, and potassium hexatitanate, K₂Ti₆O₁₃. The sample is characterized by PXRD, for which representative x-ray diffraction lines are provided in Table 27. Elemental analyses by ICP yields metals stoichiometry K_{0.26}Ti, consistent with the expected stoichiometry of potassium octatitanate, K₂Ti₈O₁₇.

TABLE 27

| 2-Θ | d(Å) | I/I₀ % |
|---|---|---|
| 11.34 | 7.80 | vs |
| 24.00 | 3.70 | w |
| 25.32 | 3.51 | vs* |
| 29.09 | 3.07 | s |
| 29.68 | 3.01 | w (sh) |
| 36.96 | 2.43 | w* |
| 37.81 | 2.38 | m* |
| 38.60 | 2.33 | w* |
| 47.76 | 1.90 | m |
| 48.04 | 1.89 | m* |
| 53.90 | 1.70 | m* |
| 55.08 | 1.67 | m* |
| 62.18 | 1.49 | w* |
| 62.68 | 1.48 | m* |
| 68.76 | 1.36 | w |

*anatase contributions

### Example C3

This reaction was carried out using a procedure disclosed herein, such as Example 24 or 25, except in the absence of an MHCA, such as d-sorbitol. A 100 ml polypropylene beaker is charged with 46 g of pre-made 7.5 M NaOH solution and placed under an overhead mixer. With vigorous mixing, 4 g of pure anatase TiO₂ powder (Sigma-Aldrich) is added, and the reaction mixture is stirred for 5 minutes. A portion of the reaction mixture is placed in a 45 ml Teflon-lined Parr vessel and digested quiescently at 200° C. for 24 hrs at autogenous pressure. The solid product is isolated by centrifugation, washed with deionized water, and dried at 100° C. overnight. PXRD is used to characterize the product, and representative diffraction lines are provided in Table 28.

TABLE 28

| 2-Θ | d(Å) | I/I₀ % |
|---|---|---|
| 9.18 | 9.62 | vs |
| 10.24 | 8.63 | w (sh) |
| 18.34 | 4.83 | w |
| 24.24 | 3.67 | w |
| 28.29 | 3.15 | w (br) |
| 47.62 | 1.91 | w |
| 48.10 | 1.89 | m |

### Example C4

This reaction was carried out using a procedure disclosed herein, such as Example 24 or 25, except in the absence of an MHCA. A 100 ml polypropylene beaker is charged with 40 g of pre-made 8.5 M KOH solution and placed under an overhead mixer. With vigorous mixing, 4 g of pure anatase TiO₂ powder (Sigma-Aldrich) is added, and the reaction mixture is stirred for 5 minutes. A portion of the reaction mixture is placed in a 45 ml Teflon-lined Parr vessel and digested quiescently at 200° C. for 24 hrs at autogenous pressure. The solid product is isolated by centrifugation, washed with deionized water, and dried at 100° C. overnight. PXRD is used to characterize the product, and representative diffraction lines are provided in Table 29.

TABLE 29

| 2-Θ | d(Å) | I/I₀ % |
|---|---|---|
| 7.30 | 12.11 | w (br) |
| 11.01 | 8.03 | w (br) |
| 24.07 | 3.70 | w |
| 25.30 | 3.52 | w |
| 29.19 | 3.06 | vs (br) |
| 31.43 | 2.84 | w (sh) |
| 33.78 | 2.65 | m (br) |
| 42.62 | 2.12 | w (br) |
| 47.70 | 1.90 | s |

### Example C5

The general procedure for the synthesis of formed spray dried TiO₂ spheres provided above is used. These spheres are characterized to establish the baseline properties of the spheres before conversion to metal titanates in hydroxide media. The properties of these starting material spheres are compared to the spheres converted in hydroxide media with and without an MHCA present (Example 29). The spray dried TiO₂ spheres are characterized by PXRD, revealing a mixture of anatase and rutile TiO₂ topologies. Characteristic diffraction lines are provided in Table 30.

TABLE 30

| 2-Θ | d(Å) | I/I₀ % |
|---|---|---|
| 25.24 | 3.53 | vs (A) |
| 27.40 | 3.25 | m (R) |
| 36.04 | 2.49 | w (R) |
| 36.93 | 2.43 | w (A) |
| 37.73 | 2.38 | m (A) |
| 38.56 | 2.33 | w (A) |
| 39.19 | 2.30 | w (R) |
| 41.18 | 2.19 | w (R) |
| 43.95 | 2.06 | w (R) |
| 48.00 | 1.89 | m (A) |
| 53.89 | 1.70 | m (A) |
| 54.28 | 1.69 | m (R) |
| 55.02 | 1.67 | m (A) |
| 56.60 | 1.62 | w (R) |
| 62.09 | 1.49 | w (A) |
| 62.66 | 1.48 | m (A, R) |
| 64.04 | 1.45 | w (A) |
| 68.75 | 1.36 | w (R) |
| 68.93 | 1.36 | w (A) |
| 69.82 | 1.35 | w (A) |

A = Anatase,
R = Rutile

### Example C6

This reaction using spray dried TiO₂ spheres is carried out using a procedure disclosed herein, such as Example 24 or 25, except in the absence of an MHCA. A 100 ml polypropylene beaker is charged with 40 g of 8.5 M KOH solution and placed under an overhead mixer. With vigorous mixing, 4 g of spray dried TiO₂ spheres are added, and the reaction mixture is stirred for 5 minutes. A portion of the reaction mixture is placed in a 45 ml Teflon-lined Parr vessel and digested at 225° C. for 1-day during which it is tumbled at 30 rpm at autogenous pressure. The solid product was isolated by centrifugation, washed with deionized water, and dried at 100° C. overnight. PXRD is used to characterize the products. Representative diffraction lines for the product are provided in Table 31.

TABLE 31

| 2-Θ | d(Å) | I/I₀ % |
|---|---|---|
| 10.96 | 8.07 | vs (br) |
| 24.13 | 3.69 | w |
| 29.25 | 3.05 | vs (br) |
| 32.32 | 2.77 | m (sh) |
| 33.56 | 2.67 | m (br) |
| 42.48 | 2.13 | w (br) |
| 47.92 | 1.90 | m |
| 52.91 | 1.73 | w (br) |
| 59.29 | 1.56 | w (br) |
| 66.15 | 1.41 | w (br) |

### Example 27-SEMs of the Metal Titanate Ion Exchangers

One result in the synthesis of the metal titanates of the present disclosure is the aggregation of crystallites into rather large robust polycrystalline particulates which are often hundreds of times the size of the individual crystals. While not limited by theory, the synthetic chemistry from solution is designed to transport the metals in highly basic conditions, conditions where the metals would otherwise be present as poorly reactive gels. The additional outcome is the formation of large polycrystalline aggregates that are beneficial for Pb²⁺ remediation in the gastrointestinal tract, since they tend to be large enough to avoid absorption and resulting adverse effects.

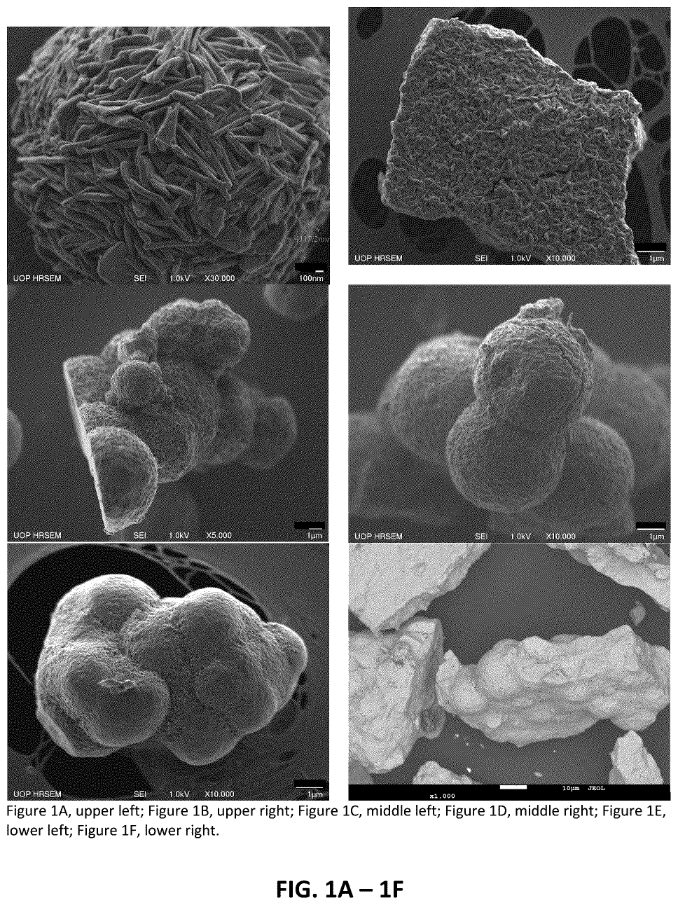

Scanning Electron Microscopy (SEM) is used to illustrate these properties, the formation of the large polycrystalline aggregates. FIG. 1A shows the product from Example 5B, a Na_{0.25}Fe_{0.20}Ti_{0.80} product prepared from solution, that appears as an aggregate of many well-formed plate-like crystals balled up in a sphere. These thin plates offer ion exchange capability since the small dimensions, which provide access to ion exchange sites more easily than if the individual crystals are large. FIG. 1B shows the same Example 5B material present as a larger slab, similarly formed of a mass of intertwined thin plates. FIGS. 1C and 1D show the SEMs of the Example 7B product, Na_{0.33}Fe_{0.38}Ti_{0.62}, another iron titanate synthesized from solution. These materials form a different morphology, namely polycrystalline aggregates composed of interpenetrating spheres of the smaller plate-like crystals. FIG. 1E shows the SEM of the Example 9 product, Na_{0.39}Zr_{0.03}Ti_{0.97}, also synthesized from homogenous solution, which also forms aggregates of interpenetrating spheres in which the spheres are less defined than in the FIG. 1C product, forming a smoother large polycrystalline aggregate. FIG. 1F shows the SEM of the K—Ti—Nb—O product of Example 11, again synthesized from solution. The observed morphology for this material is a horizontal slab of interpenetrating spheres, and again the spheres are composed of plate-like crystals. These slabs are 10 microns thick and tens of microns across, and thereby of sufficient size to avoid absorption via the gastrointestinal tract.

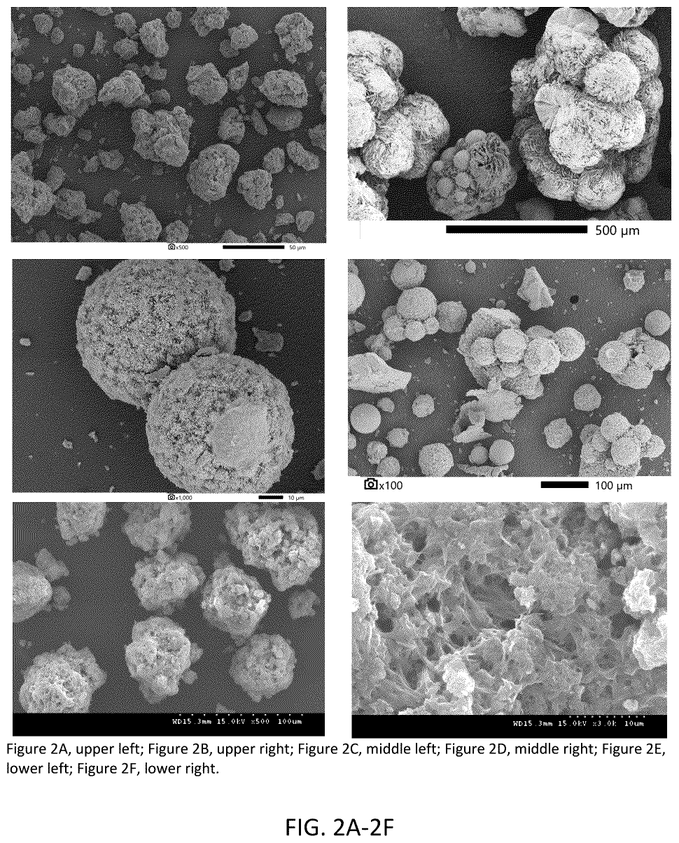

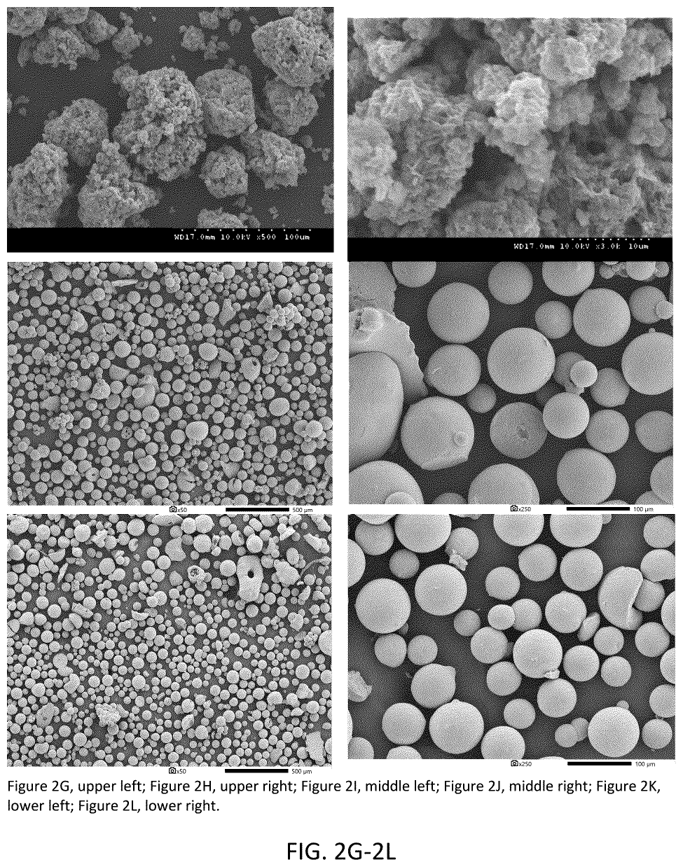

The solution chemistry approach for solubilizing metals in highly basic solution facilitates preparation of the mixed metal titanate ion exchangers of the present disclosure and provides the unanticipated benefit of forming large polycrystalline aggregates beneficial for removing Pb²⁺ from the gastrointestinal tract. Specifically, the observed attribute of a MHCA's solubilization of Ti might can, in one aspect, facilitate the chemistry of TiO₂ powders in highly basic solution and can, in an aspect, provide the particulate size benefits seen with the solution chemistry. SEMs of the products derived from TiO₂ powders are shown in FIG. 2. FIG. 2A shows the SEM of the Example 17 potassium titanate product, K_{0.26}Ti, prepared from TiO₂ powder in the presence of KOH and the MHCA catechol, 1,2-dihydroxybenzene. The SEM shows spongy polycrystalline aggregates, most of which are between 5 to tens of microns in size. FIG. 2B shows the Example 20A product resulting from the hydrothermal treatment of preformed spray dried TiO₂ spheres in the presence of a d-sorbitol/NaOH solution. The polycrystalline nature of the spheres is plainly visible showing the aggregate of plate crystals reminiscent of those observed in solution chemistry products, shown in FIGS. 1A and 1C. Further, the preformed spheres have aggregated with the NaOH/d-sorbitol hydrothermal treatment, forming larger aggregates often well over 100 microns in size. FIGS. 2C and 2D are SEMs of the Example 21A product, which is a Na—Fe—Ti—O composition derived from preformed spray dried TiO₂ spheres hydrothermally treated with an iron nitrate/citric acid/d-sorbitol/NaOH solution. The polycrystalline nature of the intact sphere is visible, and the spheres range from about 20-70 microns in size. Many of the spheres are individuals, while some are interpenetrating. FIGS. 2E and 2F show SEMs of the Example 24A product, K_{0.40}Ti, derived from hydrothermally treating TiO₂ powder in the presence of KOH/d-sorbitol solution. FIG. 2E is a field view showing spongy polycrystalline aggregates about 50 microns in size. FIG. 2F shows the macroporous nature of the aggregate particle in a close-up view. The procedure used to prepare the Example 24A product was repeated an additional 10 times resulting in the Example 24E composite sample formed from combining these batches. FIGS. 2G and 2H depict SEMs of the composite sample, K_{0.30}Ti, which exhibit very similar characteristics to the single batch of Example 24A, including the aggregates tens of microns in size (FIG. 2G) and a sponge-like macroporous network (FIG. 2H). FIGS. 21 and 2J are SEMs of the Example 25D composite sample derived by combining three batches of preformed spray dried TiO₂ spheres hydrothermally converted to K_{0.30}Ti in the presence of KOH/d-sorbitol solution. The field view in FIG. 2I shows the potassium titanate spheres to be in the size range of tens of microns, while under higher magnification in FIG. 2J the spheres range from about 20-80 microns in diameter. This composite K_{0.30}Ti sphere product of Example 25D was treated with acid to make the Example 25E product, K_{0.16}Ti. FIGS. 2K and 2L, SEMs of the acid treated Example 25E product are similar in appearance to the parent as-synthesized K_{0.30}Ti sample, suggesting the formed spheres survive intact through the acid treatment process. It is evident by these examples that the hydrothermal synthesis of metal titanates in the presence of MHCAs yields large polycrystalline aggregates that greatly exceed the 3-micron size and thus may not be absorbed by the body via the gastrointestinal tract.

### Example 28

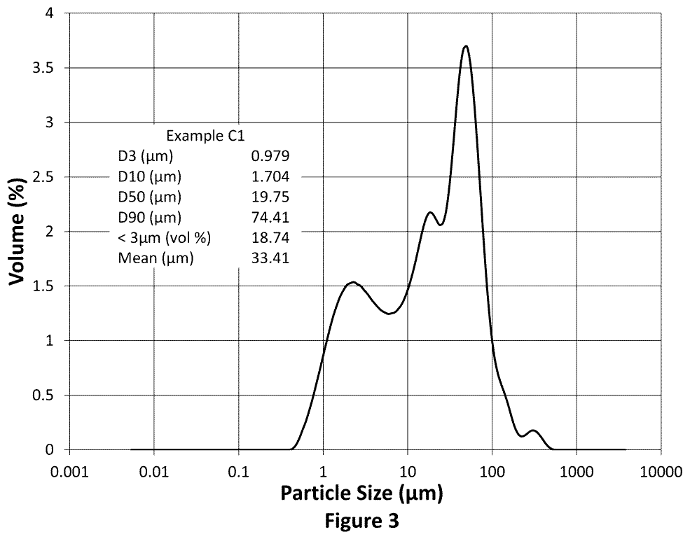

Particle Size distribution (PSD) measurements are also carried out to determine the size characteristics of selected examples as well as some of the comparative examples. Separate batches of powder samples were analyzed using light scatter diffraction techniques in a LS 13 320 XR Particle Size Analyzer. Powder samples are dispersed in water and sonicated briefly before measurement. The particle size distribution and other measured parameters are shown in FIGS. 3-8. The D (3), D (10), D (50), and D (90) values represent the particle size values in microns under which smallest 3%, 10%, 50%, and 90% of the sample occur in the particle size distribution. The D (50) value is equivalent to the Median of the particle size distribution. Without being bound by theory, particles less than 3 microns in size can be absorbed by the body, and it is thus desirable to have few of these particles, although 3 percent may be viewed as being an acceptable upper limit. In the figures, the parameter <3 μm (vol %) gives the volume percent of the sample having a particle size less than 3 μm. The Mean or average particle size is also reported for each sample.

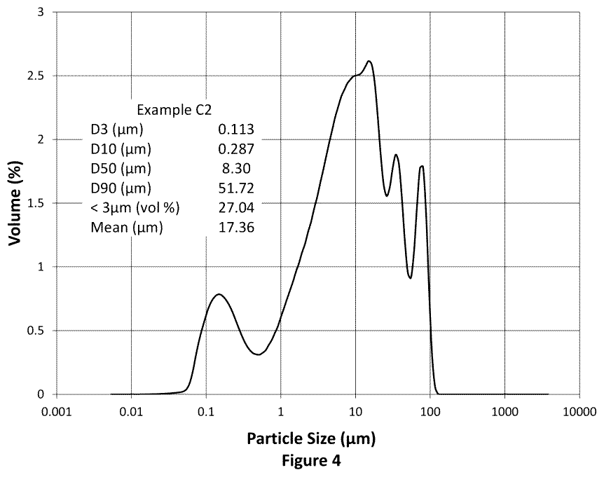

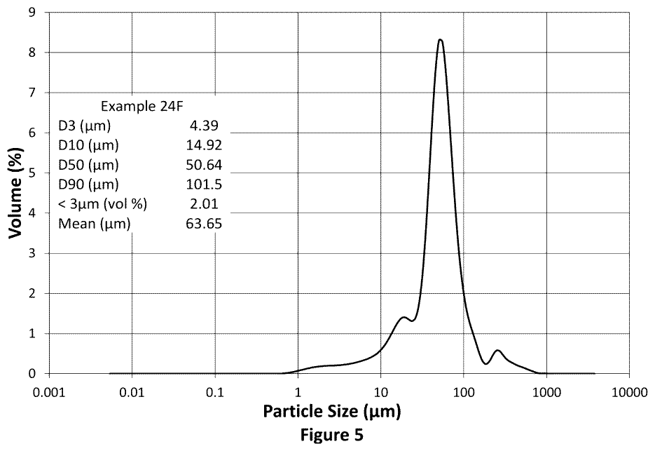

FIG. 3 shows the particle size distribution of comparative Example C1, an Allied-Signal sodium nonatitanate sample disclosed in U.S. Pat. No. 11,964,266, Example 4, for removal of Pb²⁺ and other metals from bodily fluids. For this sample, D (3) shows that 3 volume percent of the sample has a particle size less than 0.98 μm and that 18.74 volume percent of the sample has a particle size less than 3 microns. Similarly, FIG. 4 shows the particle size distribution of comparative Example C2, a Honeywell potassium octatitanate product disclosed in U.S. Pat. No. 11,964,266, Example 17, for removal of Pb²⁺ and other metals from bodily fluids. For this sample, D (3) shows that 3 volume percent of the sample has a particle size less than 0.11 micron, and 27.04 volume percent of the sample is less than 3 microns in particle size. For each of the comparative Example C1 and Example C2 materials, the particle size distribution is multipeaked and broad, having relatively significant fractions of sample at particle sizes ranging over two orders of magnitude. The broad distributions are characterized by mean particle sizes that are nearly twice that of the median particle sizes. In addition, for each of these samples, the large fractions of sample with particle size less than 3 μm, 18.74 and 27.04 volume percent for the Example C1 and C2 samples, respectively, makes these samples unacceptable for treatment involving the gastrointestinal tract, because these small particles could be absorbed by the body, causing adverse effects. In contrast, like the comparative Examples C1 and C2, the Example 24F material that is the subject of FIG. 5 is derived from the hydrothermal treatment of TiO₂ powder in the presence of alkali hydroxide solution, specifically KOH, but also contains the MHCA d-sorbitol. Visually, the particle size distribution is more uniform falling under one main peak with two minor peaks, two minor portions of sample with larger and smaller particle sizes. The D (3) shows that 3 volume percent of the Example 24F sample has a particle size less than 4.39 μm in size and that 2.01 volume percent of the sample is less than 3 μm in particle size, making this sample much more acceptable for use in gastrointestinal tract-based treatments. The mean particle size exceeds the median particle size by approximately 25% in contrast to the large differences seen for the mean and median particle sizes for Examples C1 and C2. In the presence of MHCA d-sorbitol, the median particle size for the Example 24F material of 50.64 μm is much larger than the 8.302 μm seen for the Example C2 sample, which is a potassium octatitanate also prepared in KOH solution from TiO₂ powder. The presence of the d-sorbitol provides a more uniform particle size distribution with a larger particle size more suited for gastrointestinal-based treatments.

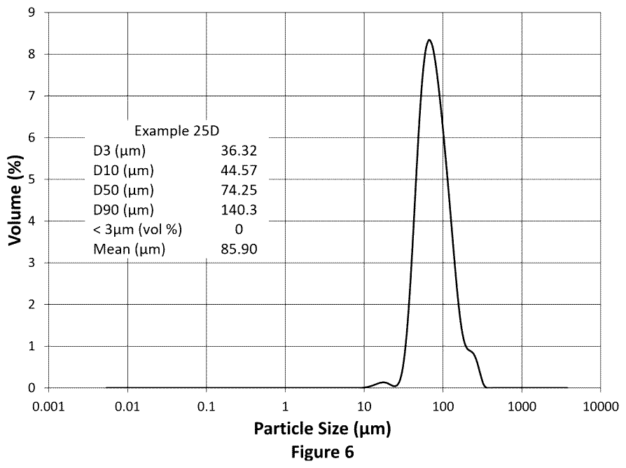

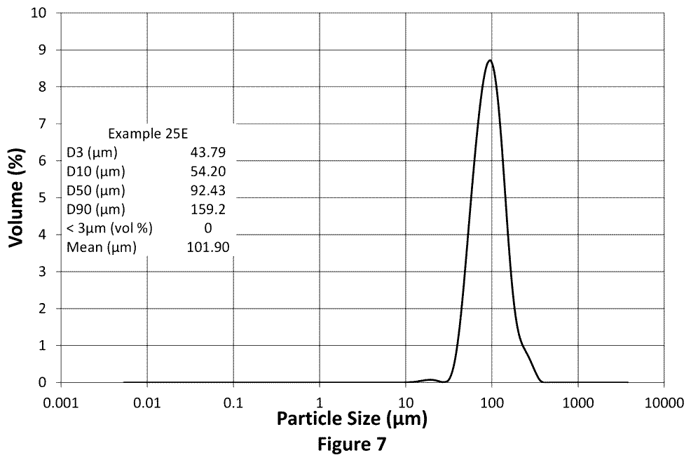

FIG. 6 shows the particle size distribution for the composite Example 25D material derived from a formed starting material, spray dried TiO₂ spheres, treated with KOH/d-sorbitol solution to make the metal titanate ion exchanger. Most notably the particle distribution is single-peaked with a shoulder at larger particle size and a very small peak at lower particle size. The strategy of starting with spray dried TiO₂ spheres to make the metal titanates dramatically changes the particle size distribution, particularly with regard to smaller particles. For this sample, D (3) is 36.32 μm, and the smallest 3 volume percent of the particles are less than this particle size. There is no measurable fraction of the particles less than 3 μm in size, making this material a good candidate for use in gastrointestinal treatment. Likewise, FIG. 7 shows the particle size distribution for the Example 25E sample, which is derived by acid treatment of the Example 25D composite material discussed in FIG. 6. Without being bound by theory, in an aspect, it may be desirable to change the cation form of the ion exchanger used to treat a patient if the patient has certain sensitivities, restrictions, or conditions, such as hyperkalemia or calcium deficiency. The Example 25E material has undergone an acid treatment process, and FIG. 7 shows that for the Example 25E sample D (3) is 43.79 μm, the smallest 3 volume percent is less than this size. Like the parent sample, Example 25D, there is no measurable fraction of the particles less than 3 μm in size. Thus, the metal titanate spheres survive the ion exchange process without breaking up to form undesirable smaller particles.

FIG. 8 shows the particle size distribution for the Example 26 product derived from a nano-sized pure anatase phase TiO₂ powder, digested in d-sorbitol/KOH solution at 200° C. for 24 hr. The PSD for this sample is single-peaked, with a slight shoulder on the smaller particle side, and quite narrow and symmetric as the mean particle size, 54.77 μm is nearly the same as the median particle size, 54.08 μm. Despite fabrication from a nano-sized TiO₂ reagent, the D (3) for this material is 17.17 μm and the volume percent of sample less than 3 μm is 0.55%. This product is uniform and suitable for treating the gastrointestinal tract.

### Example 29

MHCAs are used to synthesize the metal titanate ion exchangers of the present disclosure, providing macroporosity imparted to the products. A macroporous product with a high surface area enhances the immediate availability of ion exchange sites. BET surface areas of some metal titanate ion exchangers and those of the Comparative Examples are listed in Table 32.

TABLE 32

| Example | Description | S_{BET} (m²/g) |
|---|---|---|
| 24E | Conversion of TiO₂ powder in | 197 |
| | KOH/d-sorbitol, Composite Sample | |
| 24F | Acid-treated Composite Sample | 236 |
| 25D | Conversion of TiO₂ spheres in | 203 |
| | KOH/d-sorbitol solution | |
| C1 | Prior Art Sodium Nonatitanate from | 44 |
| | Allied Signal | |
| C2 | Prior Art Potassium Octatitanate from | 8 |
| | Honeywell | |
| C3 | Conversion of TiO₂ powder in NaOH | 74 |
| | solution, no complexing agent | |
| C4 | Conversion of TiO₂ powder in KOH | 100 |
| | solution, no complexing agent | |
| C5 | TiO₂ spray dried sphere (preformed | 46 |
| | starting material) | |
| C6 | Conversion of TiO₂ sphere in KOH | 119 |
| | solution, no complexing agent | |

Example 24E is the composite sample resulting from combining 11-2 L preparations of TiO₂ powder hydrothermally converted in KOH/d-sorbitol solution. The surface area is 197 m²/g while that of the acid extracted composite, Example 24F, is even higher at 236 m²/g. Similarly, the Example 25D composite sample of TiO₂ spheres hydrothermally treated in KOH/d-sorbitol solution exhibits a surface area of 203 m²/g. The prior art titanates from Example C1 and Example C2 used for Pb²⁺ remediation of bodily fluids in U.S. Pat. No. 11,964,266, sodium nonatitanate and potassium octatitanate, respectively, which are not synthesized in the presence of MHCAs, show surface areas of 44 and 8 m²/g, respectively, which are significantly less than the materials of the present disclosure. For comparison, to illustrate the benefit of synthesis in the presence of MHCAs, surface areas were collected for Examples C3 and C4, which are conversions of TiO₂ powder in the presence of NaOH and KOH solutions, respectively, with no MHCAs present. The surface areas of these materials were 74 m²/g (Example C3) and 100 m²/g (Example C4), still half or less than the surface area of metal titanates of Examples 24E, 24F and 25D that are synthesized in the presence of the d-sorbitol complexing agent. Surface areas are also determined for the starting material TiO₂ spheres (Example C5), and the same TiO₂ spheres converted to a titanate in the presence of KOH, but without an MHCA present (Example C6). The surface area of the Example C5 TiO₂ sphere starting material is 46 m²/g, while after conversion in KOH solution with no d-sorbitol (Example C6), the surface area is increased to 119 m²/g. The surface area of the Example C6 material is less than 60% of the corresponding Example 25D sample converted in KOH/d-sorbitol solution. In these direct comparisons with complexing agent-free Examples C3, C4, C5, and C6, the conversions in the presence of d-sorbitol complexing agent generate macroporous materials with significantly higher surface areas.

### Example 30

The metal titanate materials disclosed in Examples 1-25 and Comparative Examples C1-C6 are tested to determine their ability to adsorb Pb²⁺, Mg²⁺, Ca²⁺, K⁺ and Na⁺ ions from a test solution by determining the distributions (K_{d}) for each of the metals between adsorption on the solid vs. remaining in the solution state. The test solution was prepared from the source compounds as provided in Table 33:

TABLE 33

| | [Mⁿ⁺] |
|---|---|
| Compound | ppm |
| Ca(NO₃)₂•4H₂O | 25 |
| CH₃COOH | 2,498 |
| KNO₃ | 300 |
| Mg(NO₃)₂•6H₂O | 25 |
| NACH₃COO | 1,171 |
| NaNO₃ | 1,829 |
| Pb standard | 15 |

The concentrations in the table are those for the metal component, e.g., 25 ppm Ca²⁺, 300 ppm K⁺, etc. The total Na⁺ content was targeted at 3000 ppm, arising from NaNO₃ and sodium acetate. The solution is buffered with acetic acid/sodium acetate buffer; an acetic acid concentration is targeted at 2500 ppm. while the acetic acid/sodium acetate ratio is targeted at 2.9. A standard 10,000 ppm Pb solution is used to bring the Pb concentration to a targeted value of 15 ppm Pb.

For the test, 0.1000 g solid metal titanate was mixed with 100 ml of the test solution giving a liquid/solid ratio L/S=1000. The uptake experiments are carried out in 125 ml HDPE bottles placed in a New Brunswick Innova 40 Incubator Orbital Shaker operating at 120 rpm at 25° C. for a period of 2-2.5 hr. After the exposure, 10 ml aliquots of the ion exchanged solutions as well as untreated starting feed are filtered through 0.2 μm Thermo Scientific™ Target 2™ Nylon/GMF filters. The elemental analysis of Pb is conducted using ICP-MS (Perkin Elmer NexION 300D) by taking 0.1000 ml of the solution and diluting them to 200 ml. Indium is added as the internal standard for the counts, while Sc and Bi are added as additional quality monitors. Two milliliters of nitric acid are added to the final solution as well. The detection level for Pb is 0.003 ppm or 3 ppb.

The K_{d} value for the distribution coefficient of metals between solution and solid is calculated using the following formula:

$${K}_{d} ( mL / g ) = \frac{\begin{matrix}( V ) & ( Ac )\end{matrix}}{\begin{matrix}( W ) & ( Sc )\end{matrix}} 1$$

where: V=volume of bodily fluid simulant (mL)
Ac=concentration of cation absorbed on ion-exchanger (g/mL)
W=mass of ion-exchanger evaluated (g)
Sc=concentration of cation in post reaction supernate (g/mL).

The Pb²⁺ uptake test results for the materials of each Example are given in Table 34A, and are expressed in terms of K_{d}, the distribution coefficient. Similarly, uptake results for Na⁺, K⁺, Mg²⁺ and Ca²⁺ are given for selected samples in Table 34B.

TABLE 34A

| | Pb²⁺ K_{d} |
|---|---|
| Example | (mL/g) |
| 1A | >4,965,000 |
| 1B | >4,965,000 |
| 2 | >4,965,000 |
| 3A | 1,610,000 |
| 3B | 1,862,000 |
| 4A | 1,163,000 |
| 4B | 930,300 |
| 5A | 1,752,000 |
| 5B | 29,100 |
| 6A | 1,107,000 |
| 6B | 127,600 |
| 7A | 111,400 |
| 7B | 516,900 |
| 8 | 231,400 |
| 9 | >4,965,000 |
| 10 | >4,799,000 |
| 11 | 479,000 |
| 12 | 73,600 |
| 13 | 1,821,000 |
| 14A | 3,624,000 |
| 14B | 744,000 |
| 15A | 5,466,000 |
| 15B | 3,279,000 |
| 16 | >5,165,000 |
| 17 | 1,426,000 |
| 18 | 128,600 |
| 19A | 260,400 |
| 19B | 73,900 |
| 20A | 110,900 |
| 20B | 89,960 |
| 21A | 200,300 |
| 21B | 164,900 |
| 22 | 456,600 |
| 23A | 1,257,000 |
| 23B | 443,100 |
| 24A | 1,589,000 |
| 24B | 396,500 |
| 24C | 1,012,000 |
| 24D | 505,700 |
| 24E | 809,500 |
| 24F | 495,800 |
| 25A | 1,099,000 |
| 25B | 699,000 |
| 25C | 961,500 |
| 25D | 967,800 |
| 25E | 321,900 |
| 26 | 1,539,000 |
| C1 | 258,700 |
| C2 | 504,300 |
| C5 | 1,074 |
| C6 | 296,100 |

TABLE 34B
Na⁺, K⁺, Mg²⁺, Ca²⁺ Uptake distribution coefficients, K_{d}, (mL/g)

| Example | Na⁺ | K⁺ | Mg²⁺ | Ca²⁺ |
|---|---|---|---|---|
| 24F | — | — | 16 | 91 |
| 25E | 13 | — | 68 | 166 |

The metal titanates of the present disclosure exhibit robust Pb²⁺ uptake from the test solution. The first 15 Examples in Table 34A are the materials prepared from homogenous solution. Two different sodium titanate structures (Examples 1A and 1B) and a potassium titanate composition (Example 2) prepared from solution removed Pb²⁺ below detectable levels, exhibiting a K_{d} of nearly 5,000,000 mL/g representing 99.98% of Pb²: removed. A scale-up of a sodium titanate using less complexing agent and NaOH (Example 3A) is also effective, and this effectiveness is improved for the Mg² ion-exchanged version (Example 3B), having a different structure. Modification of the metal titanate structure by perturbing the preparation via Li addition in the presence of a large amount of K⁺, illustrating the powerful structure directing influence of Li, yields products that have a Pb²⁺ distribution coefficient, K_{d}, of about 1,000,000 for both the low temperature (Example 4A) and high temperature (Example 4B) structures. The sodium iron titanates of Example 5 illustrate that products derived from the same reaction mixture can exhibit variable levels of Pb²⁺ uptake. Indeed, the low temperature structure (Example 5A) for the sodium iron titanate exhibits a Pb²⁺ K_{d}=1,752,000 (removal of 99.94% Pb²⁺), while the distribution coefficient for the high temperature structure (Example 5B) exhibits reduced performance, K_{d}=29,100 (removal of 96.67% Pb²). The same trend is observed in Example 6 for sodium iron titanates synthesized from solution at lower hydroxide levels, where the lower temperature structure (Example 6A) outperforms the high temperature structure (Example 6B) with K_{d} values varying by nearly a factor of 10. Increasing the iron in the sodium iron titanate reverses this trend as the high temperature product (Example 7B) exhibits a higher Pb²⁺ K_{d} by nearly a factor of 5 over the low temperature product (Example 7A). The potassium iron titanate of Example 8 removed 99.57% of Pb², which is below the performance level of the low temperature sodium iron titanates of Examples 5A and 6A. The performance of the Examples 9 and 10 materials, sodium zirconium titanate and sodium niobium titanate respectively, are noted as removing Pb²⁺ to undetectable levels in the test, exhibiting Pb²⁺ K_{d} of at least 4,800,000. The K_{d} observed for a potassium niobium titanate, Example 11, is about 10 times less than the minimum possible K_{d} for the Example 10 sodium niobium titanate, but the Example 11 potassium niobium titanate still removed 99.80% of the Pb²⁺ in the test. The performance of the Example 12 material, a potassium iron titanate with 50% more iron content than the Example 8 potassium iron titanate, followed the trends already observed, namely potassium iron titanate performance is not at the level of the sodium iron titanate performance (compare Table 34A Examples 7A/7B and Example 12 high iron results) and higher iron content titanates (Examples 7B and 12 for sodium and potassium iron titanates, respectively) are not as effective as the corresponding lower iron content titanates (Examples 5A and 6A for sodium iron titanates and Example 8 for potassium iron titanate). Still, the Example 12 potassium iron titanate with high iron removes 98.67% of the Pb²⁺ from the test solution. Examples 13-15 demonstrate the incorporation of M²⁺ to make metal titanates where M²⁺=Co²⁺ and Mn²⁺, which is enabled by the novel chemistry approach disclosed in this application. The performance of the two sodium cobalt titanates, Examples 13 and 14A in Table 34A, are noted as having Pb²⁺ K_{d}s of U.S. Pat. Nos. 1,821,000 and 3,264,000, respectively. When the Example 14A sodium cobalt titanate is ion exchanged with Mg² (Example 14B), the performance is somewhat lower, opposite of what is observed for the Mg²-exchanged sodium titanate of Example 3B, which shows improved performance over the parent sodium titanate (Example 3A). Still, the Example 14B material exhibits a Pb²⁺ K_{d}=744,000, with 99.87% Pb²⁺ removed from the test solution. The sodium manganese titanate of Example 15 is noted as having a K_{d}=5,466,000, the highest measured value for Pb²⁺ K_{d}, corresponding to 99.98% of Pb²⁺ removed from the test solution. The high pH chemistry in homogenous solution, which is enabled using multiple complexing agents including MHCAs, provides a wide variety of alkali metal titanate compositions that remove efficiently Pb²⁺ from the test solution.

Examples 16-25 in Table 34A are the Pb²⁺ uptake test results for materials derived from TiO₂ powder and spray dried TiO₂ spheres. Unlike the solution chemistry derived Examples 1-15, these materials are often derived from TiO₂ powder in the presence of hydroxide and a least one complexing agent, an MHCA, the same reaction media required in the syntheses of all metal titanates of the present disclosure. When additional metals are incorporated into the metal titanate ion exchanger, such as Fe or Co, an additional complexing agent is required, such as citric acid and, in some cases, hydrogen peroxide. Example 16 illustrates the synthesis of a sodium titanate from freshly precipitated titanium oxyhydroxide in the presence of NaOH/catechol solution where the MHCA is catechol, also known as 1,2-dihydroxybenzene. This material, Na_{0.37}Ti, removed Pb²⁺ below the detectable level of 3 ppb, giving a distribution coefficient Pb²⁺ K_{d}>5,165,000. The Example 17 material, K_{0.26}Ti, is also derived in the presence of catechol but using KOH solution and TiO₂ powder. This material also provides efficient Pb²⁺ uptake, having a Pb²⁺ K_{d}=1,426,000, corresponding to removal of 99.93% of the Pb². Example 18 illustrates the synthesis of a mixed metal titanate from TiO₂ powder, the composition K_{0.31}Fe_{0.18}Ti_{0.82}, made using citric acid and MHCA d-sorbitol to keep Fe³⁺ in solution in the concentrated KOH solution. Pb²⁺ K_{d}=128,600 for this material, corresponding to removal of 99.23% of the Pb²⁺ from the test solution. Similarly, in NaOH solution, again in the presence of Fe³⁺, citric acid and d-sorbitol, the complexing agents used to keep Fe³⁺ soluble in the strong NaOH solution, the sodium iron titanates Na_{0.33} Fe_{0.18}Ti_{0.82} (Example 19A) and Na_{0.46}Fe_{0.17}Ti_{0.83} (Example 19B) with different structures are prepared from TiO₂ powder at 200° C. after 1 d and 4 d, respectively. The performance of the Example 19A material, Pb². K_{d}=260,400, is more robust than that of the Example 18 potassium iron titanate derived from KOH and the Example 19B material prepared under harsher conditions, Pb²⁺ K_{d}=73,900, which represents removal of 98.7% of the lead from the test solution. This variation in Pb²⁺ uptake performance depending on synthesis conditions mirrors that seen for the solution derived sodium iron titanates of Examples 5A/5B and 6A/6B. The high crystallinity of the Example 20A sodium titanate formed from spray dried TiO₂ spheres is evident in FIG. 2B, and this material exhibits a Pb²⁺ K_{d}=100,900, while after acid extraction, Example 20B, the Pb²⁺ K_{d} fell to 89,960. An iron-containing metal titanate sphere, formed from TiO₂ spheres and an iron/citric acid/d-sorbitol/NaOH solution, Example 21A, exhibits a Pb²⁺ K_{d}=200,300, nearly twice the value seen for the Example 20A sodium titanate sphere, while acid extraction (Example 21B) also reduces the Pb²⁺ K_{d} to 164,900, which represents 99.4% of Pb²⁺ removed from the test solution. Like Example 20A, Example 22 is a highly crystalline sodium titanate prepared from spray dried TiO₂ spheres and d-sorbitol/NaOH solution having been agitated by tumbling rather than a static reaction, and has a Pb²⁺ K_{d}=456,600, which is more efficient than the Example 20A material. The potassium titanate of Example 23A is also prepared by tumbling spray dried TiO₂ spheres in a KOH/d-sorbitol solution. The observed Pb²⁺ K_{d}=1,257,000 is greater than those observed for the corresponding sodium titanate spheres of Examples 20A and 22. Upon treatment with acid, which yields the Example 23B product, Pb². K_{d}=443,100 is observed. The Example 24A product comes from a single 2 L batch scale-up of TiO₂ powder treated with KOH/d-sorbitol in a stirred reactor. This material exhibits a Pb²⁺ K_{d}=1,589,000. Acid treatment of this material yields the Example 24B product, which has a Pb²⁺ K_{d}=396,500, similar to that of the acid-treated Example 23B product. A portion of the Example 24A product is calcined at 350° C. for 2 hr in clean dry air, a process that may be used for annealing the metal titanate exchanger with a binder or just annealing the metal titanate ion exchanger itself. The calcined Example 24C product exhibits a Pb²⁺ K_{d}=1,012,000, showing that this material survives typical conditions used in binding and still performs well. Acid extraction of the calcined Example 24C product yields the Example 24D product, which also tests well with Pb²⁺ K_{d}=505,700. The stirred 2 L reaction used to prepare the Example 24A product is carried out 10 more times and the products are combined into a single composite product, Example 24E. The composite sample has a Pb². K_{d}=809,500, which corresponds to 99.9% removal of the Pb²⁺ from the test solution. The acid extraction product of the composite sample, Example 24F, exhibits Pb²⁺ K_{d}=495,800, lower than that of the parent product. In a similar manner to the Example 24 series derived from TiO₂ powder, the synthesis starting with spray dried TiO₂ spheres is also scaled up in KOH/d-sorbitol solution, the Example 25 series. Examples 25A, 25B, and 25C are products of individual 2 L static reactions, exhibiting Pb²⁺ K_{d}s of 1,099,000, 699,000 and 961,500, respectively. These products are combined to form the Example 25D composite sample, which exhibits a Pb²⁺ K_{d}=967,800, a slightly improved Pb²⁺ uptake performance than the TiO₂ powder-derived composite Example 24E sample. Acid extraction of the composite Example 25D product yields the Example 25E product with a reduced Pb²⁺ K_{d}=321,900, corresponding to 99.7% removal of Pb²⁺ from the test solution. The Example 26 material prepared from nano-sized anatase titania reagent not only exhibited a uniform particle size distribution, but performed excellently in Pb²⁺ uptake, exhibiting Pb²⁺ K_{d}=1,539,000. In general, this family of TiO₂ powder-derived metal titanate ion exchangers perform well in Pb²⁺ uptake.

Examples C1-C6 are the comparative examples that are provided to illustrate the advances put forth in this disclosure. Example C1 is a sodium nonatitanate product, a sample from Allied Signal that is disclosed to remediate Pb²⁺ from bodily fluids in U.S. Pat. No. 11,964,266, Example 4. This material, which is derived from treating titania with NaOH, is tested for this disclosure and yields a Pb²⁺ K_{d}=258,700. In contrast, a titania powder converted to a sodium titanate in the presence of NaOH and an MHCA agent such as catechol, as in Example 16 of the present application, exhibits an improved Pb²⁺ K_{d}>5,165,000. As seen in Examples 27 and 28, the Example C1 product has low surface area (44 m²/g) and an unacceptable particle size distribution, rendering it unsafe in humans. Likewise, Example C2 is a potassium octatitanate product from Honeywell that is disclosed for remediation of Pb²⁺ from bodily fluids in U.S. Pat. No. 11,964,266, Example 17. This material yields a Pb²⁺ K_{d}=504,300. In contrast, the Example 24E composite sample of eleven 2 L preparations, a potassium titanate derived from treating TiO₂ powder in the presence of KOH/d-sorbitol solution, has an improved Pb²⁺ K_{d}=809,500. As seen in Examples 27 and 28, the Example C2 potassium octatitanate has a lower surface area, 8 m²/g vs. 196 m²/g for the Example 24E potassium titanate composite, and an unacceptable particle size distribution for gastrointestinal-based treatment (27.0 volume percent of sample less than 3μ). Examples C5 and C6 show the benefits of using an MHCA when applied to the hydrothermal conversion of TiO₂ spray dried spheres. The Example C5 spray dried TiO₂ sphere is of the same type used to make the Example 25 products, a measure of background performance in Pb²⁺ uptake, and yields a Pb²⁺ K_{d}=1074, which is limited performance. In Example C6, the Example C5 spray dried TiO₂ spheres are converted to potassium titanate in the presence of potassium hydroxide only, similar to the preparation methods used for Examples C1 and C2, with no MHCA present. The Pb². K_{d}=296,100 for this material falls short of the Pb²⁺ K_{d}=967,800 for the Example 25D composite sample of converted titania spheres formed in the presence of KOH and additionally, the MHCA d-sorbitol. In addition, the Example C6 material exhibits a surface area of 119 m²/g, whereas the surface area is much higher, 203 m²/g, for the potassium titanate derived from the KOH/d-sorbitol treatment.

Table 34B shows the distribution coefficients (K_{d}s) for Na⁺, K⁺, Mg²⁺, Ca²⁺ uptake for two K⁺—H⁺ titanate ion-exchangers from examples 24F and 25E. The K_{d}s are in the range of tens to hundreds, not the hundreds of thousands or millions seen for Pb²⁺ uptake. The low affinity of these ion exchangers for biologically important cations is a great advantage as other physiological processes are not interrupted during a treatment for elevated Pb²⁺ in the body. Ideally, the ion exchangers utilized for Pb²⁺ removal will “minimally disrupt” the concentrations of other cations in the body.

### Example 31

### Competitive Pb²⁺ Uptake

The Pb²⁺ uptake studies disclosed in Example 30 involve the uptake of free Pb²⁺ ion from the test solutions. Without being limited by theory, uptake of Pb²⁺ in bodily fluids is a more challenging case compared to uptake of Pb²⁺ in test solutions, as Pb²⁺ may be complexed by components present in the blood or the gastrointestinal tract. One component that has been identified is L-glutathione, L-GSH, which is widely distributed in animal tissues, plant cells and microorganisms (See J. Biol. Chem., 263, 17205-17208, 1988). A study of workers that had been exposed to lead via occupation showed with increasing BLLs, the blood glutathione levels as well as the activities of glutathione utilizing enzymes were reduced (See Science of The Total Environment, 170 (1-2), 95-100, 1995). The nature of Pb²⁺-glutathione complex formation under biologically relevant conditions at L-GSH/Pb²⁺ ratios varying from 2-10 was examined, revealing at least three different complexes that possibly may be present in the body (See Inorg. Chem., 51 (11), 6285-6298, 2012). Here, a competitive Pb²⁺ adsorption study is carried out to determine the efficacy of metal titanate ion exchangers to uptake Pb²⁺ in the presence of L-GSH. The test procedure is the same as disclosed in Example 30, except that the test solutions are modified in two ways. First, two test solutions are modified by adding L-GSH to attain L-GSH/Pb²⁺ ratios of 10 and 1. Second, for the L-GSH/Pb²⁺=1 tests, the adsorption portion is carried out at 37° C., i.e., body temperature. The results are provided in Table 35.

TABLE 35

| Sample | L-GSH/Pb²⁺ | Pb ICP (ppm) | K_{d} (mL/g) |
|---|---|---|---|
| Original Feed | 0 | 15.0 | — |
| Ex. 24F | 0 | 0.028 | 534,700 |
| L-GSH | 10 | 15.1 | — |
| Modified Feed | | | |
| Ex. 24F | 10 | 4.51 | 2,326 |
| Ex. 24F | 1 | 0.031 | 482,900 |
| Ex. 25E | 1 | 0.029 | 516,200 |

To establish a baseline, the Example 24F material, the acid-treated composite sample derived from hydrothermal conversion of titania powder in KOH/d-sorbitol solution, is retested without L-GSH present, yielding K_{d}=534,700. The L-GSH containing feed with L-GSH/Pb²⁺⁼¹⁰ is also tested in the absence of any adsorbent to ensure that the Pb²⁺-glutathione complex is stable during the adsorption process, which is confirmed. In the presence of the 10-fold excess of L-GSH complexing agent, the Example 24F material still removes 70% of the Pb²⁺ from the test solution, K_{d}=2,326. This result shows that the ion exchanger is capable of competing for Pb²⁺ when it is complexed. When L-GSH/Pb²⁺=1, the results for both the Example 24F and 25E (acid-treated composite of metal titanate spheres) materials are similar to those with no L-GSH at all. In view of this, the ion exchangers can perform in environments where Pb²⁺ is complexed.

What is claimed is:

1. A particulate metal titanate ion exchanger having an empirical formula on an anhydrous basis of:
AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85,
wherein the particulate metal titanate ion exchanger having been synthesized in the presence of at least one multihydroxyl-containing complexing agent (MHCA), and wherein the particulate metal titanate ion exchanger exhibits a median particle size of greater than 3 microns (μm).

2. The ion exchanger of claim 1, wherein the particulate metal titanate ion exchanger is an acid-treated particulate metal titanate ion exchanger.

3. The ion exchanger of claim 1, wherein A is potassium ion, hydronium ion, or a mixture thereof.

4. The ion exchanger of claim 1, wherein the particulate metal titanate ion exchanger is a polycrystalline aggregate metal titanate ion exchanger.

5. The ion exchanger of claim 1, wherein the particulate metal titanate ion exchanger is macroporous.

6. The ion exchanger of claim 1, wherein the particulate metal titanate ion exchanger has spherical morphology.

7. The ion exchanger of claim 1, wherein the particulate metal titanate ion exchanger has amorphous morphology.

8. The ion exchanger of claim 1, wherein the particulate metal titanate ion exchanger is a powder.

9. The ion exchanger of claim 1, wherein the median particle size is between 25 to 125 microns (μm).

10. The ion exchanger of claim 1, wherein less than 3% of the particles of the particulate metal titanate ion exchanger have a particle size of less than 3 microns (μm).

11. The ion exchanger of claim 1, wherein the particulate metal titanate ion exchanger has
a particle size distribution d₁₀ value of between about 5 microns (μm) and about 70 μm;
a particle size distribution d₅₀ value of between about 25 μm and about 125 μm; and
a particle size distribution d₉₀ value of between about 55 μm and about 185 μm.

12. The ion exchanger of claim 1, wherein the particulate metal titanate ion exchanger is stable in a liquid environment at a pH of 1-2; substantially insoluble at a pH range of 1-13; or both.

13. The ion exchanger of claim 1, wherein the particulate metal titanate ion exchanger has a Brunauer-Emmett-Teller (BET) surface area that is greater than 150 square meters per gram (m²/g), greater than 200 m²/g, or greater than 230 m²/g.

14. The ion exchanger of claim 1, wherein the particulate metal titanate ion exchanger has a distribution coefficient (K_{d}) for Pb²⁺ of between about 50,000 to about 5,500,000 milliliters per gram (mL/g) in solution.

15. The ion exchanger of claim 1, wherein the at least one MHCA is selected from the group consisting of a sugar alcohol, a sugar, an aromatic compound, and any combination thereof, optionally wherein the at least one MHCA is d-sorbitol.

16. The ion exchanger of claim 1, wherein x is 1 and y is 0, and m is between 0.10 to 0.50.

17. A macroporous particulate titanate ion exchanger having an empirical formula on an anhydrous basis of:
AₘTiO_{z}
wherein
A is an exchangeable cation selected from the group consisting of potassium ion, hydronium ion, and a mixture thereof, “m” is the mole ratio of A to Ti and has a value from 0.10 to 0.60; and “z” is the mole ratio of O to Ti and has a value from 2.05 to 2.60,
wherein the macroporous titanate ion exchanger having been synthesized in the presence of a multihydroxyl-containing complexing agent (MHCA) that is d-sorbitol, wherein the macroporous particulate titanate ion exchanger exhibits a median particle size that is between 25 to 125 microns (μm), wherein less than 3.0% of the particles of the macroporous particulate titanate ion exchanger have a particle size less than 3 microns (μm), and wherein the macroporous particulate titanate ion exchanger has a Brunauer-Emmett-Teller (BET) surface area of at least 150 square meters per gram (m²/g).

18. The ion exchanger of claim 17, wherein the particulate metal titanate ion exchanger is an acid-treated particulate metal titanate ion exchanger.

19. The ion exchanger of claim 17, wherein the macroporous particulate titanate ion exchanger has amorphous morphology.

20. The ion exchanger of claim 17, wherein the macroporous particulate titanate ion exchanger has any one or more of:
a particle size distribution d₁₀ value of between about 5 microns (μm) and about 45 μm;
a particle size distribution d₅₀ value of between about 25 μm and about 75 μm; and
a particle size distribution d₉₀ value of between about 55 μm and about 140 μm.

21. The ion exchanger of claim 17, wherein the macroporous particulate titanate ion exchanger exhibits a median particle size that is between 25 to 125 microns (μm), wherein less than 0.5% of the particles of the macroporous particulate titanate ion exchanger have a particle size less than 3 microns (μm).

22. The ion exchanger of claim 21, wherein the particulate metal titanate ion exchanger is an acid-treated particulate metal titanate ion exchanger.

23. The ion exchanger of claim 21, wherein the macroporous particulate titanate ion exchanger has spherical morphology.

24. The ion exchanger of claim 21, wherein the macroporous particulate titanate ion exchanger has
a particle size distribution d₁₀ value of between about 30 microns (μm) and about 70 μm;
a particle size distribution d₅₀ value of between about 55 μm and about 125 μm; and
a particle size distribution d₉₀ value of between about 120 μm and about 180 μm.

25. A method for selectively removing Pb²⁺ toxins from gastrointestinal fluid, the process comprising contacting the fluid containing the toxins with a particulate metal titanate ion exchanger, resulting in an ion exchanged ion exchanger and thereby removing the Pb²⁺ toxins from the fluid, the particulate metal titanate ion exchanger having an empirical formula on an anhydrous basis of:
AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85,
wherein the metal titanate ion exchanger having been synthesized in the presence of at least one multihydroxyl-containing complexing agent (MHCA), wherein the particulate metal titanate ion exchanger exhibits a median particle size of greater than 3 microns (μm), and wherein the particulate metal titanate ion exchanger minimally disrupts the levels of any one or more ions selected from Na⁺, Mg²⁺, K⁺, and Ca^{2+.}

26. The method of claim 25, wherein the Pb²⁺ toxins are sequestered within the ion exchanged ion exchanger after the contacting.

27. An intracorporeal process for removing Pb²⁺ toxins from gastrointestinal fluid, the process comprising contacting the fluid containing the toxins with a particulate metal titanate ion exchanger resulting in an ion exchanged ion exchanger thereby removing the toxins from the fluid, the particulate metal titanate ion exchanger having an empirical formula on an anhydrous basis of:
AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85,
wherein the particulate metal titanate ion exchanger having been synthesized in the presence of at least one multihydroxyl-containing complexing agent (MHCA).

28. A process for preparing a particulate metal titanate ion exchanger, the particulate metal titanate ion exchanger having an empirical formula on an anhydrous basis of:
AₘTiₓM_{y}O_{z}
wherein
A is an exchangeable cation selected from the group consisting of potassium ion, sodium ion, lithium ion, calcium ion, magnesium ion, hydronium ion or mixtures thereof; M is at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85,
the process comprising the steps of
(a) forming a reaction mixture comprising reactive sources of A, Ti, at least one multihydroxyl-containing complexing agent (MHCA), M, hydrogen peroxide, a complexing agent (C), and water, and
(b) heating the reaction mixture at a temperature of about 85° C. to about 225° C. for a time period of 0.5 to 30 days to form the particulate metal titanate ion exchanger,
wherein the reaction mixture comprises a composition expressed in terms of mole ratios of the oxides of:
p A₂O: a TiO₂:b MO_{q/2}:c H₂O₂:d MHCA:e C:f H₂O
wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000.

29. The process of claim 28 where the source of MHCA is d-sorbitol, mannitol, xylitol, catechol, fructose, glucose, and mixtures thereof.

30. The process of claim 28 where the source of C is citric acid, tartaric acid, EDTA, bipyridine and mixtures thereof.

31. The process of claim 28 where the initial reaction mixture contains hydrogen peroxide, complexing agent C, multihydroxyl-containing complexing agent MHCA, Ti(OiPr)₄, and optionally M and is a homogenous solution.

32. The process of claim 28 where the Ti source is TiO₂ powder, nano-sized TiO₂ powder, or preformed spray dried TiO₂ spheres, optionally wherein the source of Ti further comprises Ti(OiPr)₄.

33. The process of claim 28 where the Ti source is TiO₂ powder, nano-sized TiO₂, or preformed spray dried TiO₂ spheres, MHCA is d-sorbitol, M is Fe, Mn, Co, Zr or mixtures thereof, and the C source is citric acid.

34. The process of claim 28 where the main Ti source is TiO₂ powder, nano-sized TiO₂, or preformed spray dried TiO₂ spheres, optionally wherein the source of Ti further comprises Ti(OiPr)₄, MHCA is d-sorbitol, hydrogen peroxide source is 30 wt. % hydrogen peroxide, M is Fe, Mn, Co, Zr, Nb or mixtures thereof, and the C source is citric acid.

35. A method for manufacturing a tablet or capsule or for oral administration, the tablet or capsule comprising a particulate metal titanate ion exchanger having an empirical formula on an anhydrous basis of:
AₘTiₓM_{y}O_{z}
wherein
A is an exchangeable cation selected from the group consisting of potassium ion, sodium ion, lithium ion, calcium ion, magnesium ion, hydronium ion or mixtures thereof; M is optionally at least one framework metal selected from niobium (5+), zirconium (4+), tin (4+), iron (3+), iron (2+), cobalt (2+), and manganese (2+); “m” is the mole ratio of A to total metal (total metal=Ti+M) and has a value from 0.10 to 0.60; “x” is the mole fraction of total metal that is Ti and has a value from 0.5 to 1; “y” is the mole fraction of total metal that is M and has a value from zero to 0.5, wherein x+y=1; and “z” is the mole ratio of O to total metal and has a value from 1.55 to 2.85, wherein the particulate metal titanate ion exchanger exhibits a median particle size of greater than 3 microns (μm),
the method comprising the steps of:
(a) forming a reaction mixture comprising reactive sources of A, Ti, at least one multihydroxyl-containing complexing agent (MHCA), M, hydrogen peroxide, a complexing agent (C), and water,
(b) heating the reaction mixture for a period of time to form a metal titanate ion exchanger,
(c) treating the synthesized metal titanate ion exchanger via acid extraction and/or ion exchange with alkali metals, alkaline earth metals, or mixtures thereof, to form the particulate metal titanate ion exchanger with the desired composition.
(d) optionally admix the metal titanate ion exchanger with one or more pharmaceutically acceptable adjuvants, diluents or carriers to form a metal titanate ion exchanger medicament,
(e) forming a capsule or tablet comprising the particulate metal titanate ion exchanger wherein the reaction mixture comprises a composition expressed in terms of mole ratios of the oxides of:
p A₂O: a TiO₂:b MO_{q/2}:c H₂O₂:d MHCA:e C:f H₂O
wherein “p” has a value from about 4 to 40; “a” has a value from about 0.5 to 1; “b” has a value from 0 to 0.5, a+b=1; “q” is the charge on M and has a value from 2 to 5; “c” has a value from 0 to 6; “d” has a value from 0.2 to 4; “e” has a value of 0 to 4; and “f” has a value from 20 to 1000.
